(19) United States
(12) Patent Application Publication
(10) Pub. No.: US 2023/0152322 A1
(43) Pub. Date: May 18, 2023

(54) METHODS FOR DIAGNOSING AND TREATING UVEAL MELANOMA

(71) Applicant: The Board of Trustees of the Leland Stanford Junior University, Stanford, CA (US)

(72) Inventors: Vinit Mahajan, Palo Alto, CA (US); Gabriel Velez, Palo Alto, CA (US); Prithvi Mruthyunjaya, Redwood City, CA (US)

(21) Appl. No.: 17/796,179

(22) PCT Filed: Jan. 29, 2021
(86) PCT No.: PCT/US2021/015830

§ 371 Date: Jul. 28, 2022

Related U.S. Application Data
(60) Provisional application No. 62/968,721, filed on Jan. 31, 2020.

(51) Int. Cl. G01N 33/574; C12Q 1/6886

(52) U.S. Cl. CPC G01N 33/57407; G01N 33/57488; C12Q 1/6886; C12Q 2600/158; C12Q 2600/156

(57) ABSTRACT

Compositions, methods, and kits are provided for diagnosing and treating uveal melanoma. In particular, biomarkers have been identified that can be used to diagnose uveal melanoma and subtype eye tumors according to their gene expression profile (GEP) class or PRAME status. These biomarkers can be used alone or in combination with one or more additional biomarkers or relevant clinical parameters in prognosis, diagnosis, or monitoring treatment of uveal melanoma.

### STATEMENT REGARDING FEDERALLY SPONSORED RESEARCH OR DEVELOPMENT

This invention was made with Government support under contract P30EY26877 awarded by the National Institutes of Health. The Government has certain rights in the invention.

### BACKGROUND OF THE INVENTION

Uveal melanoma (UM) is the most common primary intraocular tumor in adults, affecting approximately 5-11 individuals per million per year (Singh et al. Ophthalmology. 118:1881-1885; 2011 and Singh et al, Ophthalmol Clin North Am. 18:75-84, 2005). The median age of diagnosis is the sixth decade of life, with a slight male predominance and strong propensity for Caucasian patients (Raivio. Acta Ophthalmol Suppl. 133:1-64; 1977; Shammas and Blodi, Arch Ophthalmol. 95:63-69, 1977). Profound or even complete vision loss is common, since current standard of care treatments for local disease include radioactive plaque therapy, external beam radiation, laser therapy, or enucleation (Damato. Clin Experiment Ophthalmol 32:639-647, 2004). Approximately 50% of patients with UM develop metastatic disease, most commonly in the liver, with up to 85% of these patients succumbing to their visceral metastases (Bell and Wilson, Cancer Control, 11:296-303, 2004). Once confirmed to have metastatic disease, patients commonly undergo systemic chemotherapy.

Currently, patients are screened with serial body imaging (CT or MRI) every 6-12 months to identify macro-metastatic disease, which typically presents 2-4 years after primary diagnosis. However, it is suspected that micro-metastatic disease may develop up to 3-5 years prior to detection of the primary tumor, but there is no current method to detect micro-metastatic disease. It is critical to identify patients with micro-metastatic disease early, so that adjuvant systemic therapy can be employed judiciously to delay or prevent the development of clinically significant macro-metastatic disease.

A clinical diagnosis of uveal melanoma is often challenging and requires a multimodal approach. Clinical history may reveal extraocular malignancies suggestive of metastasis, but often patients with UM do not complain of symptoms. Indirect ophthalmoscopy may reveal the classic appearance of a pigmented dome-shaped or collar button-shaped tumor with an associated exudative retinal detachment, but UM may be variably pigmented or even amelanotic. Ultrasonography, both A-mode and B-mode, are critical tests which may show the classic medium to low internal reflectivity, choroidal excavation, and shadowing in the orbit. Other helpful, but nonspecific, imaging tests include fluorescein angiography, optical coherence tomography, autofluorescence, and indocyanine green angiography. Invasive techniques include radioactive phosphorus uptake tests, which are unfortunately associated with high false negatives and false positives (Shields J A. Surv Ophthamol. 1977; 21:443-63), and fine needle biopsy, which has been associated with seeding of the needle track (Augsburger et al. Ophthalmology. 92:39-49; 1985).

In addition to clinical tests, genetic biomarkers may also assist in confirming UM. Chromosomal abnormalities, including monosomy 3 (M3), gain of the long arm of chromosome 8 (8q+), deletion of chromosome 1p (1p−), and changes within chromosome 6 (6p+ or 6q−) have been associated with UM. Prognosis after diagnosis of UM can also be estimated based on the presence or absence of chromosomal abnormalities, which have thus been proposed as biomarkers. For example, M3 or 8q+ correlates with parameters of poor prognosis such as large tumor size, ciliary body involvement, and epithelioid cell type (Sisley K, et al. Genes Chromosomes Cancer. 1997; 19(1):22-8). Loss of chromosome 3 classically portends a poor prognosis and high risk of metastatic disease, with tumors having two intact copies of chromosome 3 correlating to a good prognosis (Prescher et al. Lancet. 347(9010):1222-5; 1996). 1p− was observed in metastases (Aalto et al, Invest Ophthalmol Vis Sci 42(2):313-7, 2001) and simultaneous 1p− and M3 was also associated with decreased survival (Prescher et al. Lancet. 347(9010):1222-5, 1996 N S Sisley et al. Genes Chromosomes Cancer. 19:22-28; 1997). Not all biomarkers portend a poor prognosis; 6p+ and 6q− are associated with better patient survival, likely because they rarely occur concurrently with M3.

Specific genetic mutations have also been linked with UM. Up to 95% of UMs have one of either guanine nucleotide-binding protein Q polypeptide (GNAQ) or guanine nucleotide-binding protein alpha-11 (GNA11), which are mutually exclusive mutations (Van Raamsdonk et al, Nature. 457:599-602; 2009 and Van Raamsdonk et al. NEJM. 363:2191-2199; 2010). Other less common mutations include BRCA-associated protein 1 (BAP1), splicing factor 3B subunit 1 (SF3B1) and eukaryotic translation initiation factor 1A, X-linked (EIF1AX). SF3B1, a factor involved in DNA-damage repair, is mutated between 10-21% of UM (Harbour J W, R et al. Nat Genet. 2013; 45(2):133-135.) SF3B1 mutations also often occur in UM that express preferentially expressed antigen in melanoma (PRAME), an oncogene that has been linked with class 1 tumors carrying an intermediate risk of metastasis. Conversely, mutations in EIF1AX, which codes for an initiation factor important for translation, occur mostly in nonmetastatic cases of UM and are associated with a good prognosis (Decatur et al. JAMA Ophthalmol. 134(7):728-733; 2016).

Invasive ocular tumor biopsies may provide prognostic estimates using gene expression profiles (GEP) that segregate tumors into low- (Class 1) or high- (Class 2) metastatic risk. Despite knowledge of this risk, GEP testing currently provides no effective early detection method or therapeutic targets. Thus, there is a critical unmet need to develop rapid and precise diagnostic tools and treatments for uveal melanoma. Earlier detection of metastatic UM may allow for more effective treatments and prolonged survival.

### SUMMARY OF THE INVENTION

Compositions, methods, and kits are provided for diagnosing and treating uveal melanoma. In particular, biomarkers have been identified that can be used to diagnose uveal melanoma and subtype eye tumors according to their gene expression profile (GEP) class or preferentially expressed antigen in melanoma (PRAME) status. These biomarkers can be used alone or in combination with one or more additional biomarkers or relevant clinical parameters in prognosis, diagnosis, or monitoring treatment of uveal melanoma.

Biomarkers that can be used in diagnosing uveal melanoma include, without limitation, fatty acid-binding protein 1 (FABP1), granulocyte-macrophage colony-stimulating factor receptor (GM-CSF Ra), kallikrein 7 (KLK7), sialic acid-binding Ig-like lectin 6 (SIGL6), Myc proto-oncogene protein (MYC), oncostatin-M (OSM), stem cell growth factor receptor Kit (SCFR/KIT), common beta chain (CSF2RB), hepatocyte growth factor receptor (c-MET/HGFR), sirtuin-1 (SIR1), granzyme A (GRAA), myocyte-specific enhancer factor 2C (MEF2C), arginase-1 (ARGI1), fas ligand (FASLG), pancreatic prohormone (PP/PAHO), DNA (cytosine-5)-methyltransferase 3A (DNMT3A), desmoglein-3 (DSG3), autotaxin (ENPP2), galectin-9 (LEG9), and hepatocyte growth factor (HGF) as well as biomarkers listed in Tables 3, 4, 6, and 7 for subtyping uveal melanoma according to GEP class or PRAME status.

In certain embodiments, a panel of biomarkers is used for diagnosis of uveal melanoma. Biomarker panels of any size can be used in the practice of the subject methods. Biomarker panels for diagnosing uveal melanoma typically comprise at least 3 biomarkers and up to 20 biomarkers, including any number of biomarkers in between, such as 3, 4, 5, 6, 7, 8, 9, 10, 11, 12, 13, 14, 15, 16, 17, 18, 19, or 20 biomarkers. In certain embodiments, a biomarker panel comprising at least 3, or at least 4, or at least 5, or at least 6, or at least 7, or at least 8, or at least 9, or at least 10, or at least 11, or at least 12, or at least 13, or at least 14, or at least 5, or at least 16, or at least 17, or at least 18, or at least 19, or at least 20, or more biomarkers. Although smaller biomarker panels are usually more economical, larger biomarker panels (i.e., greater than 20 biomarkers) have the advantage of providing more detailed information and can also be used in the practice of the subject methods.

In some embodiments, the biomarker panel comprises or consists of all of the FABP1, GM-CSF Ra, KLK7, SIGL6, MYC, OSM, SCFR/KIT, CSF2RB, c-MET/HGFR, SIR1, GRAA, MEF2C, ARGH1, FASLG, PP/PAHO, DNMT3A, DSG3, ENPP2, LEG9, and HGF biomarkers. In some embodiments, the biomarker panel comprises or consists of the SIGL6, c-MYC, OSM, and SCFR/c-Kit biomarkers for diagnosing uveal melanoma. In some embodiments, the biomarker panel comprises or consists of the FABP1, GM-CSF Ra, and KLK7 biomarkers for distinguishing patients having a positive diagnosis for uveal melanoma from those with a negative diagnosis for uveal melanoma. In some embodiments, the biomarker panel further comprises one or more biomarkers selected from Table 3 and Table 6 for classifying the uveal melanoma as a GEP Class 1 or Class 2 uveal melanoma. In some embodiments, the biomarker panel further comprises one or more biomarkers selected from Table 4 and Table 7 for classifying the uveal melanoma as a PRAME positive uveal melanoma.

In one aspect, a method of diagnosing and treating uveal melanoma in a patient is provided, the method comprising: a) obtaining a vitreous sample from an eye of the patient; b) measuring levels of expression of one or more biomarkers selected from the group consisting of fatty acid-binding protein 1 (FABP1), granulocyte-macrophage colony-stimulating factor receptor (GM-CSF Ra), kallikrein 7 (KLK7), sialic acid-binding Ig-like lectin 6 (SIGL6), Myc proto-oncogene protein (MYC), oncostatin-M (OSM), stem cell growth factor receptor Kit (SCFR/KIT), common beta chain (CSF2RB), hepatocyte growth factor receptor (c-MET/HGFR), sirtuin-1 (SIR1), granzyme A (GRAA), myocyte-specific enhancer factor 2C (MEF2C), arginase-1 (ARGH1), fas ligand (FASLG), pancreatic prohormone (PP/PAHO), DNA (cytosine-5)-methyltransferase 3A (DNMT3A), desmoglein-3 (DSG3), autotaxin (ENPP2), galectin-9 (LEG9), and hepatocyte growth factor (HGF) in the vitreous sample, wherein differential expression of FABP1, GM-CSF Ra, KLK7, SIGL6, MYC, OSM, SCFR/KIT, CSF2RB, c-MET/HGFR, SIR1, GRAA, MEF2C, ARGH1, FASLG, PP/PAHO, DNMT3A, DSG3, ENPP2, LEG9, and HGF compared to reference value ranges for a vitreous sample from a control subject indicate that the patient has uveal melanoma; and c) treating the patient for the uveal melanoma, if the patient has a positive diagnosis for said uveal melanoma based on the levels of expression of the one or more biomarkers. In one embodiment, the uveal melanoma is uveal melanoma.

In certain embodiments, the levels of expression of the SIGL6, c-MYC, OSM, and SCFR/c-Kit biomarkers in the vitreous sample from the eye of the patient are measured, wherein increased levels of expression of the SIGL6, c-MYC, OSM, and SCFR/c-Kit biomarkers in the vitreous sample from the eye of the patient compared to reference value ranges for the biomarkers in a vitreous sample from a control subject indicate that the patient has uveal melanoma.

In certain embodiments, the levels of expression of the FABP1, GM-CSF Ra, and KLK7 biomarkers in the vitreous sample from the eye of the patient are measured, wherein decreased levels of expression of the FABP1, GM-CSF Ra, and KLK7 biomarkers in the vitreous sample from the eye of the patient compared to reference value ranges for the biomarkers in a vitreous sample from a control subject indicate that the patient has uveal melanoma.

In certain embodiments, the method further comprises classifying the uveal melanoma by gene expression profile (GEP) class and/or PRAME status if the patient has a positive diagnosis for uveal melanoma.

In certain embodiments, the method further comprising classifying the uveal melanoma by gene expression profile (GEP) class if the patient has a positive diagnosis for uveal melanoma by comparing the levels of expression of one or more biomarkers selected from Table 3 and Table 6 in the vitreous sample from the eye of the patient to reference value ranges for the one or more biomarkers obtained from one or more reference vitreous samples from one or more reference subjects having uveal melanoma that has been classified by gene expression profile (GEP) class. In some embodiments, the one or more biomarkers are selected from the group consisting of oncostatin M (OSM), colony stimulating factor 2 common beta chain (CSF2RB), GM-CSF Ra, FABP1, kallikrein 7, oligodendrocyte-myelin glycoprotein (OMgp), sirtuin 1, siglec-6, myocyte-specific enhancer factor 2C (MEF2C), arginase-1, DNA (cytosine-5)-methyltransferase 3A (DNMT3A), and heparin-binding EGF-like growth factor (HB-EGF). In some embodiments, the one or more biomarkers comprise or consist of oncostatin M (OSM), colony stimulating factor 2 common beta chain (CSF2RB), GM-CSF Ra, FABP1, kallikrein 7, oligodendrocyte-myelin glycoprotein (OMgp), sirtuin 1, siglec-6, myocyte-specific enhancer factor 2C (MEF2C), arginase-1, DNA (cytosine-5)-methyltransferase 3A (DNMT3A), and heparin-binding EGF-like growth factor (HB-EGF). In some embodiments, the one or more biomarkers comprise or consist of colony stimulating factor 2 common beta chain (CSF2RB), hepatocyte growth factor receptor (c-MET/HGFR), sirtuin-1, granzyme A, myocyte-specific enhancer factor 2C (MEF2C), arginase-1, Fas ligand (FASL), pancreatic prohormone (PP), and DNA (cytosine-5)-methyltransferase 3A (DNMT3A), wherein increased levels of expression of colony stimulating factor 2 common beta chain (CSF2RB), hepatocyte growth factor receptor (c-MET/HGFR), sirtuin-1, granzyme A, myocyte-specific enhancer factor 2C (MEF2C), arginase-1, Fas ligand (FASL), pancreatic prohormone (PP), and DNA (cytosine-5)-methyltransferase 3A (DNMT3A) compared to reference value ranges for a vitreous sample from a control subject indicate that the patient has GEP Class 2 uveal melanoma.

In certain embodiments, the method further comprises classifying the uveal melanoma by PRAME status if the patient has a positive diagnosis for uveal melanoma by comparing the levels of expression of one or more biomarkers selected from Table 4 and Table 7 in the vitreous sample from the eye of the patient to reference value ranges for the one or more biomarkers obtained from one or more reference vitreous samples from one or more reference subjects having uveal melanoma that has been classified by PRAME status. In some embodiments, the one or more biomarkers are selected from the group consisting of desmoglein-3, GM-CSF Ra, FABP1, kallikrein 7, and siglec-6. In some embodiments, the one or more biomarkers comprise or consist of desmoglein-3, GM-CSF Ra, FABP1, kallikrein 7, and siglec-6. In some embodiments, the one or more biomarkers comprise or consist of desmoglein-3, autotaxin, galectin-9, hepatocyte growth factor (HGF), neogenin (NEO1), and pro-low-density lipoprotein receptor-related protein 1 (LRP1), wherein increased levels of expression of desmoglein-3, autotaxin, galectin-9, hepatocyte growth factor (HGF), neogenin (NEO1), and pro-low-density lipoprotein receptor-related protein 1 (LRP1) indicate that the patient has PRAME positive uveal melanoma.

In certain embodiments, the patient has been diagnosed with idiopathic uveitis.

In certain embodiments, the method further comprises detecting leukocytes in the vitreous humor or active chorioretinal inflammation in the patient.

In certain embodiments, the patient is treated for the uveal melanoma by administering adjuvant systemic therapy, radioactive plaque therapy, external beam proton therapy, laser therapy, enucleation, evisceration, exenteration, iridectomy, choroidectomy, iridocyclectomy, eyewall resection, chemotherapy, brachytherapy, transpupillary thermotherapy, resection of the eye tumor, gamma knife stereotactic radiosurgery, or a combination thereof

In certain embodiments, measuring the level of expression of a biomarker comprises measuring a level of expression of a protein. For example, levels of a biomarker protein may be measured by a method including, but not limited to, mass spectrometry, tandem mass spectrometry, liquid chromatography, liquid chromatography-tandem mass spectrometry (LC-MS/MS), NMR, an enzyme-linked immunosorbent assay (ELISA), a radioimmunoassay (RIA), an immunofluorescent assay (IFA), immunohistochemistry, fluorescence-activated cell sorting (FACS), or a Western Blot.

In certain embodiments, the method further comprises performing ultrasonography, fluorescein angiography, optical coherence tomography, autofluorescence, indocyanine green angiography, or a radioactive phosphorus uptake test on the eye.

In certain embodiments, the method further comprises genotyping the patient to determine if the patient has one or more chromosomal abnormalities linked to uveal melanoma such as, but not limited to, monosomy 3 (M3), gain of long arm of chromosome 8 (8q+), deletion of chromosome 1p (1p−), and changes within chromosome 6 (6p+ or 6q−).

In another aspect, a method of subtyping uveal melanoma and determining risk of metastasis is provided, the method comprising: a) obtaining a vitreous sample from an eye of a patient who has uveal melanoma; b) measuring levels of expression of one or more biomarkers selected from the group consisting of colony stimulating factor 2 common beta chain (CSF2RB), hepatocyte growth factor receptor (c-MET/HGFR), sirtuin-1, granzyme A, myocyte-specific enhancer factor 2C (MEF2C), arginase-1, Fas ligand (FASL), pancreatic prohormone (PP), and DNA (cytosine-5)-methyltransferase 3A (DNMT3A), wherein increased levels of expression of the one or more biomarkers selected from the group consisting of CSF2RB, c-MET/HGFR, sirtuin-1, granzyme A, MEF2C, arginase-1, FASL, PP, DNMT3A in the vitreous sample from the patient compared to reference value ranges for the biomarkers from a vitreous sample from a control subject indicate that the patient has GEP class 2 uveal melanoma and is at risk of metastasis; and c) measuring levels of expression of one or more biomarkers selected from the group consisting of desmoglein-3, autotaxin, galectin-9, hepatocyte growth factor (HGF), neogenin (NEO1), and pro-low-density lipoprotein receptor-related protein 1 (LRP1) wherein increased levels of expression of the one or more biomarkers selected from the group consisting of desmoglein-3, autotaxin, galectin-9, HGF, NEO1, and LRP1 in the vitreous sample from the eye of the patient compared to reference value ranges for the biomarkers from a vitreous sample from a control subject indicate that the patient has PRAME positive uveal melanoma and is at risk of metastasis.

In certain embodiments, the method further comprises administering adjuvant systemic therapy, radiotherapy, or performing surgery if the patient is diagnosed with GEP class 2 uveal melanoma or PRAME positive uveal melanoma indicating that the patient has a high risk of metastasis.

In certain embodiments, the method further comprises measuring levels of one or more additional biomarkers selected from Table 3, Table 4, Table 6, and Table 7.

In another aspect, a method of monitoring uveal melanoma in a patient is provided, the method comprising: a) obtaining a first vitreous sample from an eye of the patient at a first time point and a second vitreous sample from the eye of the subject later at a second time point; b) measuring one or more biomarkers in the first vitreous sample and the second vitreous sample, wherein the biomarkers are selected from the group consisting of SIGL6, c-MYC, OSM, SCFR/c-Kit, FABP1, KLK7, GM-CSF Ra, and serpin 1; and c) analyzing the levels of expression of the one or more biomarkers in conjunction with respective reference value ranges for said biomarkers, wherein detection of increased levels of expression of SIGL6, c-MYC, OSM, and SCFR/c-Kit and decreased levels of expression of FABP1, KLK7, GM-CSF Ra, and serpin 1 in the second vitreous sample compared to the first vitreous sample indicate that the patient is worsening, and detection of decreased levels of expression of SIGL6, c-MYC, OSM, and SCFR/c-Kit and increased levels of expression of FABP1, KLK7, GM-CSF Ra, and serpin 1 in the second vitreous sample compared to the first vitreous sample indicate that the patient is improving.

In another aspect, a method of monitoring efficacy of a treatment of a patient for uveal melanoma is provided, the method comprising: a) obtaining a first vitreous sample from the patient before the patient undergoes the treatment and a second vitreous sample from the subject after the patient undergoes the treatment; b) measuring one or more biomarkers in the first vitreous sample and the second vitreous sample, wherein the biomarkers are selected from the group consisting of SIGL6, c-MYC, OSM, SCFR/c-Kit, FABP1, KLK7, GM-CSF Ra, and serpin 1; and c) evaluating the efficacy of the treatment, wherein detection of increased levels of expression of SIGL6, c-MYC, OSM, and SCFR/c-Kit and decreased levels of expression of FABP1, KLK7, GM-CSF Ra, and serpin 1 in the second vitreous sample compared to the first vitreous sample indicate that the patient is worsening or not responding to the treatment, and detection of decreased levels of expression of SIGL6, c-MYC, OSM, and SCFR/c-Kit and increased levels of expression of FABP1, KLK7, GM-CSF Ra, and serpin 1 in the second vitreous sample compared to the first vitreous sample indicate that the patient is improving. In certain embodiments, the method further comprises altering the treatment if the patient is worsening or not responding to the treatment.

In another aspect, a kit is provided for diagnosing uveal melanoma, the kit comprising agents for detecting at least 3 biomarkers selected from the group consisting of fatty acid-binding protein 1 (FABP1), granulocyte-macrophage colony-stimulating factor receptor (GM-CSF Ra), kallikrein 7 (KLK7), sialic acid-binding Ig-like lectin 6 (SIGL6), Myc proto-oncogene protein (MYC), oncostatin-M (OSM), stem cell growth factor receptor Kit (SCFR/KIT), common beta chain (CSF2RB), hepatocyte growth factor receptor (c-MET/HGFR), sirtuin-1 (SIR1), granzyme A (GRAA), myocyte-specific enhancer factor 2C (MEF2C), arginase-1 (ARGH1), fas ligand (FASLG), pancreatic prohormone (PP/PAHO), DNA (cytosine-5)-methyltransferase 3A (DNMT3A), desmoglein-3 (DSG3), autotaxin (ENPP2), galectin-9 (LEG9), and hepatocyte growth factor (HGF).

In certain embodiments, the kit comprises agents for detecting all of the FABP1, GM-CSF Ra, KLK7, SIGL6, MYC, OSM, SCFR/KIT, CSF2RB, c-MET/HGFR, SIR1, GRAA, MEF2C, ARGH1, FASLG, PP/PAHO, DNMT3A, DSG3, ENPP2, LEG9, and HGF biomarkers.

In certain embodiments, the kit further comprises agents for detecting one or more biomarkers selected from Table 3, Table 4, Table 6, or Table 7.

In certain embodiments, the kit comprises agents for detecting oncostatin M (OSM), colony stimulating factor 2 common beta chain (CSF2RB), GM-CSF Ra, FABP1, kallikrein 7, oligodendrocyte-myelin glycoprotein (OMgp), sirtuin 1, siglec-6, myocyte-specific enhancer factor 2C (MEF2C), arginase-1, DNA (cytosine-5)-methyltransferase 3A (DNMT3A), and heparin-binding EGF-like growth factor (HB-EGF).

In certain embodiments, the kit comprises agents for detecting colony stimulating factor 2 common beta chain (CSF2RB), hepatocyte growth factor receptor (c-MET/HGFR), sirtuin-1, granzyme A, myocyte-specific enhancer factor 2C (MEF2C), arginase-1, Fas ligand (FASL), pancreatic prohormone (PP), and DNA (cytosine-5)-methyltransferase 3A (DNMT3A) compared to reference value ranges for a vitreous sample from a control subject indicate that the patient has GEP Class 2 uveal melanoma.

In certain embodiments, the kit comprises agents for detecting desmoglein-3, GM-CSF Ra, FABP1, kallikrein 7, and siglec-6.

In certain embodiments, the kit comprises agents for detecting desmoglein-3, autotaxin, galectin-9, hepatocyte growth factor (HGF), neogenin (NEO1), and pro-low-density lipoprotein receptor-related protein 1 (LRP1).

In certain embodiments, the kit further comprises reagents for performing an immunoassay.

In certain embodiments, the kit further comprises instructions for diagnosing uveal melanoma.

In another aspect, a protein selected from the group consisting of fatty acid-binding protein 1 (FABP1), granulocyte-macrophage colony-stimulating factor receptor (GM-CSF Ra), kallikrein 7 (KLK7), sialic acid-binding Ig-like lectin 6 (SIGL6), Myc proto-oncogene protein (MYC), oncostatin-M (OSM), stem cell growth factor receptor Kit (SCFR/KIT), common beta chain (CSF2RB), hepatocyte growth factor receptor (c-MET/HGFR), sirtuin-1 (SIR1), granzyme A (GRAA), myocyte-specific enhancer factor 2C (MEF2C), arginase-1 (ARGH1), fas ligand (FASLG), pancreatic prohormone (PP/PAHO), DNA (cytosine-5)-methyltransferase 3A (DNMT3A), desmoglein-3 (DSG3), autotaxin (ENPP2), galectin-9 (LEG9), and hepatocyte growth factor (HGF) for use as a biomarker in diagnosing uveal melanoma is provided. In some embodiments, the uveal melanoma is GEP class 1, GEP class 2, PRAME positive, or PRAME negative uveal melanoma.

In another aspect, a protein selected from the group consisting of colony stimulating factor 2 common beta chain (CSF2RB), hepatocyte growth factor receptor (c-MET/HGFR), sirtuin-1, granzyme A, myocyte-specific enhancer factor 2C (MEF2C), arginase-1, Fas ligand (FASL), pancreatic prohormone (PP), and DNA (cytosine-5)-methyltransferase 3A (DNMT3A) for use as a biomarker in diagnosing GEP Class 2 uveal melanoma is provided.

In another aspect, a protein selected from the group consisting of desmoglein-3, autotaxin, galectin-9, hepatocyte growth factor (HGF), neogenin (NEO1), and pro-low-density lipoprotein receptor-related protein 1 (LRP1) for use as a biomarker in diagnosing PRAME positive uveal melanoma is provided.

In another aspect, an in vitro method of diagnosing uveal melanoma is provided, the method comprising: a) obtaining a vitreous sample from an eye of the patient; and b) measuring levels of expression of at least 3 biomarkers selected from the group consisting of fatty acid-binding protein 1 (FABP1), granulocyte-macrophage colony-stimulating factor receptor (GM-CSF Ra), kallikrein 7 (KLK7), sialic acid-binding Ig-like lectin 6 (SIGL6), Myc proto-oncogene protein (MYC), oncostatin-M (OSM), stem cell growth factor receptor Kit (SCFR/KIT), common beta chain (CSF2RB), hepatocyte growth factor receptor (c-MET/HGFR), sirtuin-1 (SIR1), granzyme A (GRAA), myocyte-specific enhancer factor 2C (MEF2C), arginase-1 (ARGI1), fas ligand (FASLG), pancreatic prohormone (PP/PAHO), DNA (cytosine-5)-methyltransferase 3A (DNMT3A), desmoglein-3 (DSG3), autotaxin (ENPP2), galectin-9 (LEG9), and hepatocyte growth factor (HGF) in the vitreous sample, wherein differential expression of the FABP1, GM-CSF Ra, KLK7, SIGL6, MYC, OSM, SCFR/KIT, CSF2RB, c-MET/HGFR, SIR1, GRAA, MEF2C, ARGH1, FASLG, PP/PAHO, DNMT3A, DSG3, ENPP2, LEG9, and HGF compared to reference value ranges for a control sample indicate that the patient has the uveal melanoma.

### BRIEF DESCRIPTION OF THE DRAWINGS

FIGS. 1A-1H show Fundus photographs of uveal melanoma patients (UM) undergoing vitreous biopsy: (FIG. 1A) Fundus photograph (FP) from Case 1 showing large inferior uveal melanoma (UM) tumor associated with exudative retinal detachment (RD) OS. (FIG. 1B) Case 2 FP showing circumpapillary UM tumor with associated exudative RD OS. (FIG. 1C) Case 3 FP showing low-mid reflective macular domed UM legion and associated exudative RD OS. (FIG. 1D) Case 4 FP showing collar stud UM tumor encroaching the edge of the macula and associated exudative RD OS. (FIG. 1E) Case 5 FP showing superior nasal UM tumor and a low-domed, internally reflective lesion with mild subretinal fluid exudation OD. (FIG. 1F) Case 6 FP showing large temporal cilio-UM tumor with low-mid internal reflectivity and pulsation with associated exudative RD OS. (FIG. 1G) Case 7 FP showing peripapillary UM tumor with associated exudative RD OS. (FIG. 1H) Case 8 FP showing collar stud configuration UM with associated exudative RD OS.

FIGS. 2A-2H show B-scan ultrasonography of uveal melanoma patients (UM) undergoing vitreous biopsy: (FIG. 2A) B-scan ultrasound from Case 1 reveals a tumor size of 16.0×13.7×5.8 mm (A-scan height: 6.7 mm) OS. (FIG. 2B) Case 2 tumor size 6.0×7.0×2.5 mm OS. (FIG. 2C) Case 3 tumor size 12.7×11.2×4.8 mm (A-scan height: 4.1 mm) OS. (FIG. 2D) Case 4 tumor size 11.5×11.8×7.5 mm (A-scan height: 6.9 mm) OS. (FIG. 2E) Case 5 tumor size 11.0×9.0×4.0 mm (A-scan height: 4.1 mm) OD. (FIG. 2F) Case 6 tumor size 21.8×16.6×12.7 mm (A-scan height: 14.6 mm) OS. (FIG. 2G) Case 7 tumor size 15.6×15.9×7.3 mm (A-scan height: 7.6 mm) OS. (FIG. 2H) Case 8 tumor size 15.6×14.5×7.3 mm (with low internal reflectivity) OS.

FIGS. 3A-3B show targeted proteomic signatures differentiate molecular classes of uveal melanoma: Protein concentrations from the multiplex ELISA array were normalized to log base 2 and analyzed for differentially expressed proteins. Multi-group comparison (1-way ANOVA) followed by hierarchical heat map clustering was used to identify differentially expressed proteins in the large-scale dataset. (FIG. 3A) When comparing protein expression by GEP class, there were 46 differentially expressed proteins at the p<0.01 level. Results are represented as a heatmap and display protein expression levels on a logarithmic scale. Orange indicates high expression while dark green/black indicates low or no expression. (FIG. 3B) When comparing protein expression by PRAME status, there were 32 differentially expressed proteins at the p<0.01 level.

FIGS. 4A-4B show shotgun proteomic signatures differentiate molecular classes of uveal melanoma: Spectral counts from LC-MS/MS were normalized to log base 2 and analyzed for differentially expressed proteins. Multi-group comparison (1-way ANOVA) followed by hierarchical heat map clustering was used to identify differentially expressed proteins in the large-scale dataset. (FIG. 4A) When comparing protein expression by GEP class, there were 62 differentially expressed proteins at the p<0.01 level. Results are represented as a heatmap and display protein expression levels on a logarithmic scale. Orange indicates high expression while dark green/black indicates low or no expression. (FIG. 4B) When comparing protein expression by PRAME status, there were 36 differentially expressed proteins at the p<0.05 level.

FIGS. 5A-5T show selected uveal melanoma biomarkers for validation study: Protein expression levels for (FIG. 5A) Fatty acid binding protein 1 (FABP1), (FIG. 5B) Granulocyte-macrophage colony-stimulating factor (GM-CSF Ra), (FIG. 5C) Kallikrein 7 (KLK7), (FIG. 5D) Oncostatin M (OSM), (FIG. 5E) c-Myc, (FIG. 5F) Siglec-6, (FIG. 5G) Stem cell factor receptor (SCFR/c-Kit), (FIG. 5H) Common β chain (βc), (FIG. 5I) Hepatocyte growth factor receptor (HGFR/c-Met), (FIG. 5J) Sirtuin-1, (FIG. 5K) Granzyme A, (FIG. 5L) Myocyte-specific enhancer factor 2C (MEF2C), (FIG. 5M) Arginase-1, (FIG. 5N) DNA (cytosine-5)-methyltransferase 3A (DNMT3A), (FIG. 5O) Desmoglein-3, (FIG. 5P) Autotaxin (ENPP2), (FIG. 5Q) Hepatocyte growth factor (FIG. 5R), Galectin-9, (FIG. 5S) Fas ligand (FASL), and (FIG. 5T) Pancreatic prohormone (PP). Expression levels are measured as protein concentrations (pg/mL) from the multiplex ELISA training dataset. Results are displayed as violin plots with dotted lines indicating the median and upper and lower quartiles. Data were analyzed by 1-way ANOVA (significance set to p<0.05) followed by Tukey's multiple comparison text (n≥3 for all groups).

FIGS. 6A-6B show principal component analysis (PCA) of the proteomics data differentiates molecular classes of UM: Protein concentrations from the multiplex ELISA array were normalized to log base 2 and analyzed by PCA. Multi-group comparison (1-way ANOVA) followed by Benjamini and Hochberg FDR corrections was used to identify differentially expressed proteins in the large-scale dataset. PCA is composed of three components, x, y, and z. Each circle represents an individual patient. (FIG. 6A) When comparing protein signatures by GEP class, ANOVA identified 12 proteins at the false discovery rate (FDR) of 0.037% and adjusted p-value of 5.1e-4: Oncostatin M (OSM), common β chain (βc), GM-CSF Ra, FABP1, kallikrein 7, oligodendrocyte-myelin glycoprotein (OMgp), sirtuin 1, siglec-6, myocyte-specific enhancer factor 2C (MEF2C), arginase-1, DNA (cytosine-5)-methyltransferase 3A (DNMT3A), and heparin-binding EGF-like growth factor (HB-EGF). (FIG. 6B) When comparing protein signatures by PRAME status, ANOVA identified 5 proteins at the FDR of 0.005% and adjusted p-value of 5.3e-4: Desmoglein-3, GM-CSF Ra, FABP1, kallikrein 7, and siglec-6.

FIGS. 7A-7B show differentially expressed proteins reveal differences between uveal melanoma cases and controls: (FIG. 7A) Differentially expressed proteins (between UM and control vitreous) detected in the multiplex ELISA represented as a volcano plot. The horizontal axis (x-axis) displays the log 2 fold-change value (melanoma vs. controls) and the vertical axis (y-axis) displays the noise-adjusted signal as the −log 10 (p-value). (FIG. 7B) Differentially expressed proteins (between UM and control vitreous) detected by LC-MS/MS.

FIGS. 8A-8C show pathway analysis based on proteins measured by multiplex ELISA: (FIG. 8A) Top ten pathways represented in UM vitreous. (FIG. 8B) Top ten pathways represented by proteins significantly upregulated in GEP Class 1 (yellow) and Class 2 (cyan) vitreous. (FIG. 8C) Top ten pathways represented by proteins significantly upregulated in PRAME positive (green-cyan) and negative (red) vitreous. Pathways are ranked by their −log(p-value) obtained from the right-tailed Fisher's Exact Test.

FIGS. 9A-9C show pathway analysis based on proteins measured by LC-MS/MS: (FIG. 9 A) Top ten pathways represented in UM vitreous. (FIG. 9B) Top ten pathways represented by proteins significantly upregulated in GEP Class 1 (yellow) and Class 2 (cyan) vitreous. (FIG. 9C) Top ten pathways represented by proteins significantly upregulated in PRAME positive (green-cyan) and negative (red) vitreous. Pathways are ranked by their −log(p-value) obtained from the right-tailed Fisher's Exact Test.

FIGS. 10A-10B show comparative analysis reveals shared and distinct proteins among UM tumor classes: Comparative analysis of significantly upregulated proteins in each group (compared to controls) using Venn diagrams. (FIG. 10A) Proteins detected by multiplex ELISA and (FIG. 10B) LC-MS/MS.

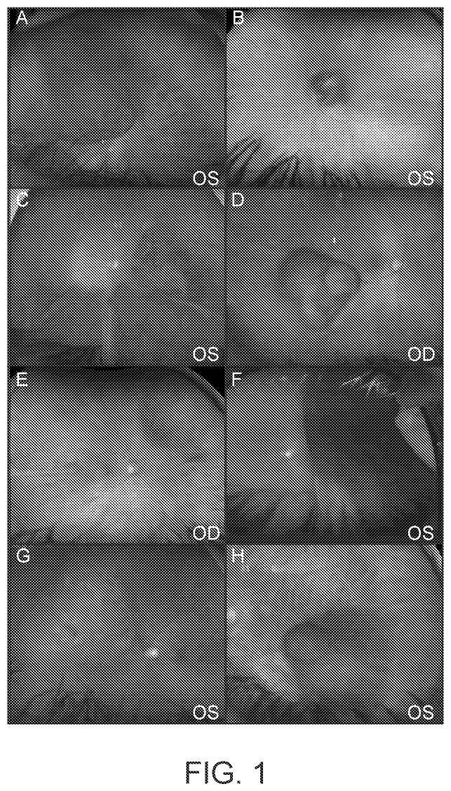

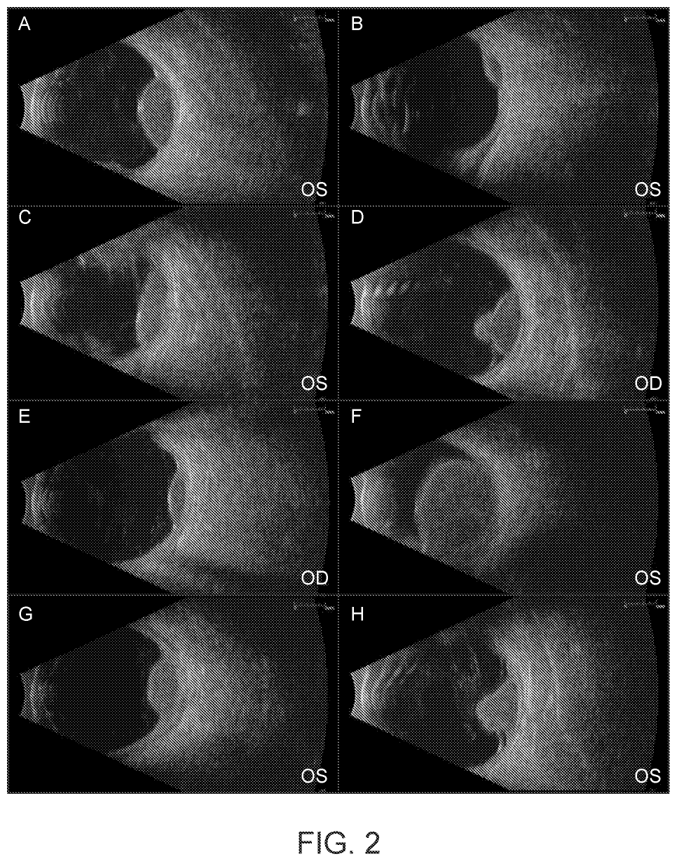

### DETAILED DESCRIPTION OF THE INVENTION

Compositions, methods, and kits are provided for diagnosing and treating uveal melanoma. In particular, biomarkers have been identified that can be used to diagnose uveal melanoma and subtype tumors according to their GEP class or PRAME status. These biomarkers can be used alone or in combination with one or more additional biomarkers or relevant clinical parameters in prognosis, diagnosis, or monitoring treatment of uveal melanoma.

Before the present compositions, methods, and kits are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a biomarker” includes a plurality of such biomarkers and reference to “the polypeptide” includes reference to one or more polypeptides and equivalents thereof, e.g., peptides or proteins known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

Biomarkers. The term “biomarker” as used herein refers to a compound, such as a protein, a mRNA, a metabolite, or a metabolic byproduct which is differentially expressed or present at different concentrations, levels or frequencies in one sample compared to another, such as a vitreous sample from patients who have uveal melanoma compared to a vitreous sample from healthy control subjects (i.e., subjects not having eye cancer). Biomarkers include, but are not limited to, fatty acid-binding protein 1 (FABP1), granulocyte-macrophage colony-stimulating factor receptor (GM-CSF Ra), kallikrein 7 (KLK7), sialic acid-binding Ig-like lectin 6 (SIGL6), Myc proto-oncogene protein (MYC), oncostatin-M (OSM), stem cell growth factor receptor Kit (SCFR/KIT), common beta chain (CSF2RB), hepatocyte growth factor receptor (c-MET/HGFR), sirtuin-1 (SIR1), granzyme A (GRAA), myocyte-specific enhancer factor 2C (MEF2C), arginase-1 (ARGI1), fas ligand (FASLG), pancreatic prohormone (PP/PAHO), DNA (cytosine-5)-methyltransferase 3A (DNMT3A), desmoglein-3 (DSG3), autotaxin (ENPP2), galectin-9 (LEG9), and hepatocyte growth factor (HGF) as well as biomarkers listed in Tables 3, 4, 6, and 7 for subtyping uveal melanoma according to GEP class or PRAME status.

In some embodiments, the concentration or level of a biomarker is determined before and after the administration of a treatment to a patient. The treatment may comprise, for example, without limitation, administering adjuvant systemic therapy, radioactive plaque therapy, external beam proton therapy, laser therapy, enucleation, evisceration, exenteration, iridectomy, choroidectomy, iridocyclectomy, eyewall resection, chemotherapy, brachytherapy, transpupillary thermotherapy, resection of the eye tumor, gamma knife stereotactic radiosurgery, or a combination thereof. The degree of change in the concentration or level of a biomarker, or lack thereof, is interpreted as an indication of whether the treatment has the desired effect (e.g., anti-tumor activity such as reducing size, growth, or number of tumors). In other words, the concentration or level of a biomarker is determined before and after the administration of the treatment to an individual, and the degree of change, or lack thereof, in the level is interpreted as an indication of whether the individual is “responsive” to the treatment.

A “reference level” or “reference value” of a biomarker means a level of the biomarker that is indicative of a particular disease state, phenotype, or predisposition to developing a particular disease state or phenotype, or lack thereof, as well as combinations of disease states, phenotypes, or predisposition to developing a particular disease state or phenotype, or lack thereof. A “positive” reference level of a biomarker means a level that is indicative of a particular disease state or phenotype. A “negative” reference level of a biomarker means a level that is indicative of a lack of a particular disease state or phenotype. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched or gender-matched so that comparisons may be made between biomarker levels in samples from subjects of a certain age or gender and reference levels for a particular disease state, phenotype, or lack thereof in a certain age or gender group). Such reference levels may also be tailored to specific techniques that are used to measure levels of biomarkers in vitreous samples (e.g., immunoassays (e.g., ELISA), mass spectrometry (e.g., LC-MS, GC-MS), tandem mass spectrometry, NMR, biochemical or enzymatic assays, PCR, microarray analysis, etc.), where the levels of biomarkers may differ based on the specific technique that is used.

A “similarity value” is a number that represents the degree of similarity between two things being compared. For example, a similarity value may be a number that indicates the overall similarity between a patient's biomarker profile using specific phenotype-related biomarkers and reference value ranges for the biomarkers in one or more control samples or a reference profile (e.g., the similarity to a “uveal melanoma GEP class 1” biomarker expression profile, a “uveal melanoma GEP class 2” biomarker expression profile, a “uveal melanoma PRAME positive” biomarker expression profile, or a “uveal melanoma PRAME negative” biomarker expression profile). The similarity value may be expressed as a similarity metric, such as a correlation coefficient, or may simply be expressed as the expression level difference, or the aggregate of the expression level differences, between levels of biomarkers in a patient sample and a control sample or reference expression profile.

The terms “quantity”, “amount”, and “level” are used interchangeably herein and may refer to an absolute quantification of a molecule or an analyte in a sample, or to a relative quantification of a molecule or analyte in a sample, i.e., relative to another value such as relative to a reference value as taught herein, or to a range of values for the biomarker. These values or ranges can be obtained from a single patient or from a group of patients.

Vitreous sample. The term “vitreous sample” with respect to an individual encompasses samples taken from the vitreous humor extracellular matrix located in the posterior chamber of the eye, such as a surgical or biopsy specimen isolated therefrom. Vitreous samples can be obtained by any suitable method such as by surgical resection or by biopsy, for example, using fine needle aspiration (FNA) or pars plana vitrectomy (PPV). The definition also includes samples that have been manipulated in any way after their procurement, such as by treatment with reagents; washed; or enriched for particular types of molecules, e.g., proteins, peptides, etc.

Obtaining and assaying a sample. The term “assaying” is used herein to include the physical steps of manipulating a vitreous sample to generate data related to the vitreous sample. As will be readily understood by one of ordinary skill in the art, a vitreous sample must be “obtained” prior to assaying the sample. Thus, the term “assaying” implies that the sample has been obtained. The terms “obtained” or “obtaining” as used herein encompass the act of receiving an extracted or isolated vitreous sample. For example, a testing facility can “obtain” a vitreous sample in the mail (or via delivery, etc.) prior to assaying the sample. In some such cases, the vitreous sample was “extracted” or “isolated” from an individual by another party prior to mailing (i.e., delivery, transfer, etc.), and then “obtained” by the testing facility upon arrival of the sample. Thus, a testing facility can obtain the sample and then assay the sample, thereby producing data related to the sample.

The terms “obtained” or “obtaining” as used herein can also include the physical extraction or isolation of a vitreous sample from a subject. Accordingly, a vitreous sample can be isolated from a subject (and thus “obtained”) by the same person or same entity that subsequently assays the sample. When a vitreous sample is “extracted” or “isolated” from a first party or entity and then transferred (e.g., delivered, mailed, etc.) to a second party, the sample was “obtained” by the first party (and also “isolated” by the first party), and then subsequently “obtained” (but not “isolated”) by the second party. Accordingly, in some embodiments, the step of obtaining does not comprise the step of isolating a vitreous sample.

In some embodiments, the step of obtaining comprises the step of isolating a vitreous sample (e.g., a pre-treatment vitreous sample, a post-treatment vitreous sample, etc.). Methods and protocols for isolating various vitreous samples will be known to one of ordinary skill in the art and any convenient method may be used to isolate a vitreous sample.

It will be understood by one of ordinary skill in the art that in some cases, it is convenient to wait until multiple samples (e.g., a pre-treatment vitreous sample and a post-treatment vitreous sample) have been obtained prior to assaying the samples. Accordingly, in some cases an isolated vitreous sample (e.g., a pre-treatment vitreous sample, a post-treatment vitreous sample, etc.) is stored until all appropriate samples have been obtained. One of ordinary skill in the art will understand how to appropriately store a variety of different types of vitreous samples and any convenient method of storage may be used (e.g., refrigeration) that is appropriate for the particular vitreous sample. In some embodiments, a pre-treatment vitreous sample is assayed prior to obtaining a post-treatment vitreous sample. In some cases, a pre-treatment vitreous sample and a post-treatment vitreous sample are assayed in parallel. In some cases, multiple different post-treatment vitreous samples and/or a pre-treatment vitreous sample are assayed in parallel. In some cases, vitreous samples are processed immediately or as soon as possible after they are obtained.

In some embodiments, the concentration (i.e., “level”), or expression level of a gene product, which may be a protein, peptide, etc., (which will be referenced herein as a biomarker), in a vitreous sample is measured (i.e., “determined”). By “expression level” (or “level”), it is meant the level of gene product (e.g., the absolute and/or normalized value determined for the RNA expression level of a biomarker or for the expression level of the encoded polypeptide, or the concentration of the protein in a vitreous sample). The term “gene product” or “expression product” are used herein to refer to the RNA transcription products (RNA transcripts, e.g., mRNA, an unspliced RNA, a splice variant mRNA, and/or a fragmented RNA) of the gene, including mRNA, and the polypeptide translation products of such RNA transcripts. A gene product can be, for example, an unspliced RNA, an mRNA, a splice variant mRNA, a microRNA, a fragmented RNA, a polypeptide, a post-translationally modified polypeptide, a splice variant polypeptide, etc.

The terms “determining”, “measuring”, “evaluating”, “assessing,” “assaying,” and “analyzing” are used interchangeably herein to refer to any form of measurement, and include determining if an element is present or not. These terms include both quantitative and/or qualitative determinations. Assaying may be relative or absolute. For example, “assaying” can be determining whether the expression level is less than or “greater than or equal to” a particular threshold, (the threshold can be pre-determined or can be determined by assaying a control sample). On the other hand, “assaying to determine the expression level” can mean determining a quantitative value (using any convenient metric) that represents the level of expression (i.e., expression level, e.g., the amount of protein and/or RNA, e.g., mRNA) of a particular biomarker. The level of expression can be expressed in arbitrary units associated with a particular assay (e.g., fluorescence units, e.g., mean fluorescence intensity (MFI)), or can be expressed as an absolute value with defined units (e.g., number of mRNA transcripts, number of protein molecules, concentration of protein, etc.). Additionally, the level of expression of a biomarker can be compared to the expression level of one or more additional genes (e.g., nucleic acids and/or their encoded proteins) to derive a normalized value that represents a normalized expression level. The specific metric (or units) chosen is not crucial as long as the same units are used (or conversion to the same units is performed) when evaluating multiple vitreous samples from the same individual (e.g., vitreous samples taken at different points in time from the same individual). This is because the units cancel when calculating a fold-change (i.e., determining a ratio) in the expression level from one vitreous sample to the next (e.g., vitreous samples taken at different points in time from the same individual).

For measuring RNA levels, the amount or level of an RNA in the sample is determined, e.g., the level of an mRNA. In some instances, the expression level of one or more additional RNAs may also be measured, and the level of biomarker expression compared to the level of the one or more additional RNAs to provide a normalized value for the biomarker expression level. Any convenient protocol for evaluating RNA levels may be employed wherein the level of one or more RNAs in the assayed sample is determined.

For measuring protein levels, the amount or level of a protein in the vitreous sample is determined. In some cases, the protein comprises a post-translational modification (e.g., phosphorylation, glycosylation) associated with regulation of activity of the protein such as by a signaling cascade, wherein the modified protein is the biomarker, and the amount of the modified protein is therefore measured. In some embodiments, an extracellular protein level is measured. For example, in some cases, the protein (i.e., polypeptide) being measured is a secreted protein (e.g., extracellular matrix protein) and the concentration can therefore be measured in vitreous fluid. In some embodiments, concentration is a relative value measured by comparing the level of one protein relative to another protein. In other embodiments the concentration is an absolute measurement of weight/volume or weight/weight.

In some instances, the concentration of one or more additional proteins may also be measured, and biomarker concentration compared to the level of the one or more additional proteins to provide a normalized value for the biomarker concentration. Any convenient protocol for evaluating protein levels may be employed wherein the level of one or more proteins in the assayed sample is determined.

While a variety of different manners of assaying for protein levels are known to one of ordinary skill in the art and any convenient method may be used, one representative and convenient type of protocol for assaying protein levels is ELISA, an antibody-based method. In ELISA and ELISA-based assays, one or more antibodies specific for the proteins of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Following incubation, the antisera-contacted surface is washed so as to remove non-immunocomplexed material. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g., urease, peroxidase, or alkaline phosphatase, which will generate a color precipitate upon incubating with an appropriate chromogenic substrate. After such incubation with the second antibody and washing to remove unbound material, the amount of label is quantified, for example by incubation with a chromogenic substrate such as urea and bromocresol purple in the case of a urease label or 2,2′-azino-di-(3-ethyl-benzthiazoline)-6-sulfonic acid (ABTS) and H₂O₂, in the case of a peroxidase label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody. The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected in a manner most appropriate for the substrate being used, for example, by removing a bead or dipstick from a reservoir, emptying or diluting a reservoir such as a microtiter plate well, or rinsing a bead, particle, chromatographic column or filter with a wash solution or solvent.

Alternatively, non-ELISA based-methods for measuring the levels of one or more proteins in a sample may be employed. Representative exemplary methods include but are not limited to antibody-based methods (e.g., immunofluorescence assay, radioimmunoassay, immunoprecipitation, Western blotting, proteomic arrays, xMAP microsphere technology (e.g., Luminex technology), immunohistochemistry, flow cytometry, and the like) as well as non-antibody-based methods (e.g., mass spectrometry or tandem mass spectrometry, liquid chromatography-tandem mass spectrometry (LC-MS/MS), NMR).

“Diagnosis” as used herein generally includes determination as to whether a subject is likely affected by a given disease, disorder or dysfunction. The skilled artisan often makes a diagnosis on the basis of one or more diagnostic indicators, i.e., a biomarker, the presence, absence, or amount of which is indicative of the presence or absence of the disease, disorder or dysfunction.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

### Additional Terms

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with uveal melanoma, those with ocular tumors, etc.) as well as those in which prevention is desired (e.g., those with increased susceptibility to eye cancer, those with an increased likelihood of eye cancer, those suspected of having eye cancer, those suspected of harboring an ocular tumor, etc.).

A therapeutic treatment is one in which the subject is inflicted prior to administration and a prophylactic treatment is one in which the subject is not inflicted prior to administration. In some embodiments, the subject has an increased likelihood of becoming inflicted or is suspected of being inflicted prior to treatment. In some embodiments, the subject is suspected of having an increased likelihood of becoming inflicted.

The term “about,” particularly in reference to a given quantity, is meant to encompass deviations of plus or minus five percent.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

A “therapeutically effective dose” or “therapeutic dose” is an amount sufficient to effect desired clinical results (i.e., achieve therapeutic efficacy). A therapeutically effective dose can be administered in one or more administrations.

By “anti-tumor activity” is intended a reduction in the rate of cell proliferation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. Such activity can be assessed using animal models.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer. Both full-length proteins and fragments thereof are encompassed by the definition. The terms also include postexpression modifications of the polypeptide, for example, phosphorylation, glycosylation, acetylation, hydroxylation, oxidation, and the like.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), and any other type of polynucleotide which is an N- or C-glycoside of a purine or pyrimidine base. There is no intended distinction in length between the terms “polynucleotide,” “oligonucleotide,” “nucleic acid” and “nucleic acid molecule,” and these terms are used interchangeably.

By “isolated” is meant, when referring to a protein, polypeptide, or peptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

The term “antibody” encompasses monoclonal antibodies, polyclonal antibodies, as well as hybrid antibodies, altered antibodies, chimeric antibodies, and humanized antibodies. The term antibody includes: hybrid (chimeric) antibody molecules (see, for example, Winter et al. (1991) Nature 349:293-299; and U.S. Pat. No. 4,816,567); bispecific antibodies, bispecific T cell engager antibodies (BiTE), trispecific antibodies, and other multispecific antibodies (see, e.g., Fan et al. (2015) J. Hematol. Oncol. 8:130, Krishnamurthy et al. (2018) Pharmacol Ther. 185:122-134), F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (scFv) (see, e.g., Huston et al. (1988) Proc Natl Acad Sci USA 85:5879-5883); nanobodies or single-domain antibodies (sdAb) (see, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

The phrase “specifically (or selectively) binds” with reference to binding of an antibody to an antigen (e.g., biomarker) refers to a binding reaction that is determinative of the presence of the antigen in a heterogeneous population of proteins and other biologics. Thus, under designated immunoassay conditions, the specified antibodies bind to a particular antigen at least two times over the background and do not substantially bind in a significant amount to other antigens present in the sample. Specific binding to an antigen under such conditions may require an antibody that is selected for its specificity for a particular antigen. For example, antibodies raised to an antigen from specific species such as rat, mouse, or human can be selected to obtain only those antibodies that are specifically immunoreactive with the antigen and not with other proteins, except for polymorphic variants and alleles. This selection may be achieved by subtracting out antibodies that cross-react with molecules from other species. A variety of immunoassay formats may be used to select antibodies specifically immunoreactive with a particular antigen. For example, solid-phase ELISA immunoassays are routinely used to select antibodies specifically immunoreactive with a protein (see, e.g., Harlow & Lane. Antibodies, A Laboratory Manual (1988), for a description of immunoassay formats and conditions that can be used to determine specific immunoreactivity). Typically, a specific or selective reaction will be at least twice background signal or noise and more typically more than 10 to 100 times background.

“Providing an analysis” is used herein to refer to the delivery of an oral or written analysis (i.e., a document, a report, etc.). A written analysis can be a printed or electronic document. A suitable analysis (e.g., an oral or written report) provides any or all of the following information: identifying information of the subject (name, age, etc.), a description of what type of vitreous sample(s) was used and/or how it was used, the technique used to assay the sample, the results of the assay (e.g., the level of the biomarker as measured and/or the fold-change of a biomarker level over time or in a post-treatment assay compared to a pre-treatment assay), the assessment as to whether the individual is determined to have uveal melanoma, and in some cases, further classification of an eye tumor by subtype (e.g., uveal melanoma GEP class and/or PRAME status), a recommendation for treatment (e.g., adjuvant systemic therapy, radioactive plaque therapy, external beam proton therapy, laser therapy, enucleation, evisceration, exenteration, iridectomy, choroidectomy, iridocyclectomy, eyewall resection, chemotherapy, brachytherapy, transpupillary thermotherapy, resection of the eye tumor, or gamma knife stereotactic radiosurgery), and/or to continue or alter therapy, a recommended strategy for additional therapy, etc. The report can be in any format including, but not limited to printed information on a suitable medium or substrate (e.g., paper); or electronic format. If in electronic format, the report can be in any computer readable medium, e.g., diskette, compact disk (CD), flash drive, and the like, on which the information has been recorded. In addition, the report may be present as a website address which may be used via the internet to access the information at a remote site.

### Biomarkers and Diagnostic Methods

Biomarkers that can be used in the practice of the subject methods include, without limitation, fatty acid-binding protein 1 (FABP1), granulocyte-macrophage colony-stimulating factor receptor (GM-CSF Ra), kallikrein 7 (KLK7), sialic acid-binding Ig-like lectin 6 (SIGL6), Myc proto-oncogene protein (MYC), oncostatin-M (OSM), stem cell growth factor receptor Kit (SCFR/KIT), common beta chain (CSF2RB), hepatocyte growth factor receptor (c-MET/HGFR), sirtuin-1 (SIR1), granzyme A (GRAA), myocyte-specific enhancer factor 2C (MEF2C), arginase-1 (ARGI1), fas ligand (FASLG), pancreatic prohormone (PP/PAHO), DNA (cytosine-5)-methyltransferase 3A (DNMT3A), desmoglein-3 (DSG3), autotaxin (ENPP2), galectin-9 (LEG9), and hepatocyte growth factor (HGF). Differential expression of these biomarkers is associated with uveal melanoma and therefore expression profiles of these biomarkers are useful for diagnosing uveal melanoma. In addition, biomarker expression profiles can be used to subtype eye tumors according to their GEP class (e.g., GEP class 1 or class 2) or PRAME status (e.g., PRAME negative or PRAME positive) using one or more biomarkers selected from Tables 3, 4, 6, and 7.

In certain embodiments, a panel of biomarkers is provided for diagnosis of uveal melanoma. Biomarker panels of any size can be used in the practice of the subject methods. Biomarker panels for diagnosing uveal melanoma typically comprise at least 3 biomarkers and up to 20 biomarkers, including any number of biomarkers in between, such as 3, 4, 5, 6, 7, 8, 9, 10, 11, 12, 13, 14, 15, 16, 17, 18, 19, or 20 biomarkers. In certain embodiments, a biomarker panel comprising at least 3, or at least 4, or at least 5, or at least 6, or at least 7, or at least 8, or at least 9, or at least 10, or at least 11, or at least 12, or at least 13, or at least 14, or at least 5, or at least 16, or at least 17, or at least 18, or at least 19, or at least 20, or more biomarkers. In some embodiments, the biomarker panel comprises or consists of all of the FABP1, GM-CSF Ra, KLK7, SIGL6, MYC, OSM, SCFR/KIT, CSF2RB, c-MET/HGFR, SIR1, GRAA, MEF2C, ARGH1, FASLG, PP/PAHO, DNMT3A, DSG3, ENPP2, LEG9, and HGF biomarkers. In some embodiments, the biomarker panel comprises or consists of the SIGL6, c-MYC, OSM, and SCFR/c-Kit biomarkers for diagnosing uveal melanoma. In some embodiments, the biomarker panel comprises or consists of the FABP1, GM-CSF Ra, and KLK7 biomarkers for distinguishing patients having a positive diagnosis for uveal melanoma from those with a negative diagnosis for uveal melanoma. In some embodiments, the biomarker panel comprises or consists of the oncostatin M (OSM), colony stimulating factor 2 common beta chain (CSF2RB), GM-CSF Ra, FABP1, kallikrein 7, oligodendrocyte-myelin glycoprotein (OMgp), sirtuin 1, siglec-6, myocyte-specific enhancer factor 2C (MEF2C), arginase-1, DNA (cytosine-5)-methyltransferase 3A (DNMT3A), and heparin-binding EGF-like growth factor (HB-EGF) biomarkers for classifying the uveal melanoma as a GEP class 1 or class 2 uveal melanoma. In some embodiments, the biomarker panel comprises or consists of the desmoglein-3, GM-CSF Ra, FABP1, kallikrein 7, and siglec-6 biomarkers for classifying the uveal melanoma as a PRAME positive or PRAME negative uveal melanoma. In certain embodiments, the biomarker panel comprises one or more biomarkers selected from Table 3, Table 4, Table 6, or Table 7. In some embodiments, the biomarker panel comprises or consists of the colony stimulating factor 2 common beta chain (CSF2RB), hepatocyte growth factor receptor (c-MET/HGFR), sirtuin-1, granzyme A, myocyte-specific enhancer factor 2C (MEF2C), arginase-1, Fas ligand (FASL), pancreatic prohormone (PP), and DNA (cytosine-5)-methyltransferase 3A (DNMT3A) biomarkers. In some embodiments, the biomarker panel comprises or consists of the desmoglein-3, autotaxin, galectin-9, hepatocyte growth factor (HGF), neogenin (NEO1), and pro-low-density lipoprotein receptor-related protein 1 (LRP1) biomarkers. Although smaller biomarker panels are usually more economical, larger biomarker panels (i.e., greater than 20 biomarkers) have the advantage of providing more detailed information and can also be used in the practice of the subject methods.

A vitreous sample comprising the expressed biomarkers is obtained from the subject. The sample is taken from the vitreous humor extracellular matrix located in the posterior chamber of the eye of the subject. A “control” sample, as used herein, refers to a vitreous sample from a subject that is not diseased. That is, a control sample is obtained from a normal or healthy subject (e.g., an individual known to not have uveal melanoma or other cancer). A vitreous sample can be obtained from a subject by conventional techniques. For example, vitreous samples can be obtained by surgical resection or by biopsy using fine needle aspiration (FNA) or pars plana vitrectomy (PPV) according to methods well known in the art.

When analyzing the levels of biomarkers in a vitreous sample from a subject, the reference value ranges used for comparison can represent the levels of one or more biomarkers in a vitreous sample from one or more subjects without uveal melanoma (i.e., normal or healthy control). Alternatively, the reference values can represent the levels of one or more biomarkers from one or more subjects with uveal melanoma, wherein similarity to the reference value ranges indicates the subject has uveal melanoma. More specifically, the reference value ranges can represent the levels of one or more biomarkers from one or more subjects with uveal melanoma (a “uveal melanoma” biomarker expression profile). If a patient is diagnosed with uveal melanoma based on similarity to a “uveal melanoma” biomarker expression profile, further comparison to reference value ranges for the levels of the biomarkers in subjects with different tumor subtypes can be used to classify eye tumors according to their GEP class, e.g., uveal melanoma GEP class 1 (a “uveal melanoma GEP class 1 biomarker expression profile”) and uveal melanoma GEP class 2 (a “uveal melanoma GEP class 2 biomarker expression profile”); and/or PRAME status, e.g., uveal melanoma PRAME positive (a “uveal melanoma PRAME positive expression profile”) and uveal melanoma PRAME negative (a “uveal melanoma PRAME negative expression profile”).

Accordingly, in one aspect, a method of diagnosing uveal melanoma in a patient is provided, the method comprising: a) obtaining a vitreous sample from an eye of the patient; and b) measuring levels of expression of one or more biomarkers selected from the group consisting of fatty acid-binding protein 1 (FABP1), granulocyte-macrophage colony-stimulating factor receptor (GM-CSF Ra), kallikrein 7 (KLK7), sialic acid-binding Ig-like lectin 6 (SIGL6), Myc proto-oncogene protein (MYC), oncostatin-M (OSM), stem cell growth factor receptor Kit (SCFR/KIT), common beta chain (CSF2RB), hepatocyte growth factor receptor (c-MET/HGFR), sirtuin-1 (SIR1), granzyme A (GRAA), myocyte-specific enhancer factor 2C (MEF2C), arginase-1 (ARGH1), fas ligand (FASLG), pancreatic prohormone (PP/PAHO), DNA (cytosine-5)-methyltransferase 3A (DNMT3A), desmoglein-3 (DSG3), autotaxin (ENPP2), galectin-9 (LEG9), and hepatocyte growth factor (HGF) in the vitreous sample, wherein differential expression of FABP1, GM-CSF Ra, KLK7, SIGL6, MYC, OSM, SCFR/KIT, CSF2RB, c-MET/HGFR, SIR1, GRAA, MEF2C, ARGH1, FASLG, PP/PAHO, DNMT3A, DSG3, ENPP2, LEG9, and HGF compared to reference value ranges for a vitreous sample from a control subject indicate that the patient has uveal melanoma.

In certain embodiments, the levels of expression of the SIGL6, c-MYC, OSM, and SCFR/c-Kit biomarkers in the vitreous sample from the eye of the patient are measured, wherein increased levels of expression of the SIGL6, c-MYC, OSM, and SCFR/c-Kit biomarkers in the vitreous sample from the eye of the patient compared to reference value ranges for the biomarkers in a vitreous sample from a control subject indicate that the patient has uveal melanoma.

In certain embodiments, the levels of expression of the FABP1, GM-CSF Ra, and KLK7 biomarkers in the vitreous sample from the eye of the patient are measured, wherein decreased levels of expression of the FABP1, GM-CSF Ra, and KLK7 biomarkers in the vitreous sample from the eye of the patient compared to reference value ranges for the biomarkers in a vitreous sample from a control subject indicate that the patient has uveal melanoma.

In certain embodiments, the method further comprises classifying the uveal melanoma by GEP class or PRAME status if the patient has a positive diagnosis for uveal melanoma. Exemplary biomarkers for determining GEP class are listed in Tables 3 and 6. Exemplary biomarkers for determining PRAME status are listed in Tables 4 and 7. In some embodiments, classifying the uveal melanoma comprises comparing the levels of expression of the one or more biomarkers from the vitreous sample from the eye of the patient to reference value ranges for the biomarkers obtained from one or more reference vitreous samples from one or more reference subjects having uveal melanoma that has been classified by GEP class or PRAME status. For example, increased levels of expression of CSF2RB, HGFR/c-MET, sirtuin-1, granzyme A, MEF2C, arginase-1, Fas L, pancreatic prohormone, and DNMT3A in the vitreous sample from the eye of the patient compared to reference value ranges for a vitreous sample from a control subject indicate that the uveal melanoma is Class 2 uveal melanoma. Increased levels of expression of desmoglein-3, autotaxin, galectin-9, and HGF in the vitreous sample from the eye of the patient compared to reference value ranges for a vitreous sample from a control subject indicate that the uveal melanoma is PRAME positive uveal melanoma.

The methods described herein may be used to determine an appropriate treatment regimen for a patient and, in particular, whether a patient should be treated for uveal melanoma. For example, a patient is selected for treatment for uveal melanoma if the patient has a positive diagnosis for uveal melanoma based on a biomarker expression profile, as described herein. The treatment for uveal melanoma may comprise, for example, administering adjuvant systemic therapy, radioactive plaque therapy, external beam proton therapy, laser therapy, enucleation, evisceration, exenteration, iridectomy, choroidectomy, iridocyclectomy, eyewall resection, chemotherapy, brachytherapy, transpupillary thermotherapy, resection of the eye tumor, gamma knife stereotactic radiosurgery, or a combination thereof. Further characterization of the tumor subtype based on expression profiling, as described herein, is useful in evaluating the severity of disease and determining prognosis. For example, patients having GEP class 1 tumors have a low risk of metastasis and patients having GEP class 2 tumors have a high risk of metastasis. In addition, patients having PRAME positive uveal melanoma have a higher risk of metastasis than patients having PRAME negative uveal melanoma. Patients identified as having a high-risk of metastasis based on GEP class (i.e., having GEP class 2 subtype) or PRAME status (i.e., having PRAME positive subtype) may be treated more aggressively, for example, with surgery, adjuvant systemic therapy, or radiotherapy, or recommended for clinical trials.

In some embodiments, the methods described herein are used for monitoring uveal melanoma in a subject. For example, a first vitreous sample can be obtained from the patient at a first time point and a second vitreous sample can be obtained from the subject at a second (later) time point. In one embodiment, uveal melanoma is monitored in the patient by measuring levels of expression of one or more biomarkers selected from the group consisting of FABP1, GM-CSF Ra, KLK7, SIGL6, c-MYC, OSM, and SCFR/c-Kit in the first vitreous sample and the second vitreous sample; and analyzing the levels of expression of the one or more biomarkers in conjunction with respective reference value ranges for the biomarkers, wherein detection of increased levels of expression of one or more biomarkers selected from the group consisting of increased levels of expression of the SIGL6, c-MYC, OSM, and SCFR/c-Kit and detection of decreased levels of expression of one or more biomarkers selected from the group consisting of FABP1, GM-CSF Ra, and KLK7 in the second vitreous sample compared to the first vitreous sample indicate that the patient is worsening (i.e., cancer is progressing), and detection of decreased levels of expression of the one or more biomarkers selected from the group consisting of SIGL6, c-MYC, OSM, and SCFR/c-Kit and detection of increased levels of expression of one or more biomarkers selected from the group consisting of FABP1, GM-CSF Ra, and KLK7 in the second vitreous sample compared to the first vitreous sample indicate that the patient is improving.

The subject methods may also be used for assaying pre-treatment and post-treatment vitreous samples obtained from an individual to determine whether the individual is responsive or not responsive to a treatment. For example, a first vitreous sample can be obtained from a subject before the subject undergoes the therapy, and a second vitreous sample can be obtained from the subject after the subject undergoes the therapy. In one embodiment, the efficacy of a treatment of a patient for uveal melanoma is monitored by measuring one or more biomarkers selected from the group consisting of FABP1, GM-CSF Ra, KLK7, SIGL6, c-MYC, OSM, and SCFR/c-Kit in the first vitreous sample and the second vitreous sample; and evaluating the efficacy of the treatment, wherein detection of increased levels of expression of the one or more biomarkers selected from the group consisting of SIGL6, c-MYC, OSM, and SCFR/c-Kit and detection of decreased levels of expression of one or more biomarkers selected from the group consisting of FABP1, GM-CSF Ra, and KLK7 in the second vitreous sample compared to the first vitreous sample indicate that the patient is worsening or not responding to the treatment, and detection of decreased levels of expression of the one or more biomarkers selected from the group consisting of SIGL6, c-MYC, OSM, and SCFR/c-Kit and detection of increased levels of expression of one or more biomarkers selected from the group consisting of FABP1, GM-CSF Ra, and KLK7 in the second vitreous sample compared to the first vitreous sample indicate that the patient is improving.

In some cases, the diagnostic methods described herein may be used by themselves or combined with medical imaging or other ophthalmology techniques for detecting ocular lesions to confirm the diagnosis and further evaluate the extent of cancerous disease (how far and where the cancer has spread) to aid in determining prognosis and evaluating optimal strategies for treatment (e.g., surgery, adjuvant therapy, radiotherapy, etc.). Exemplary medical imaging and ophthalmology techniques include, without limitation, ultrasonography, fluorescein angiography, optical coherence tomography, autofluorescence, indocyanine green angiography, and the radioactive phosphorus uptake test on the eye.

In some cases, combinations of biomarkers are used in the subject methods. In some such cases, the levels of all measured biomarkers must change (as described above) in order for the diagnosis to be made. In some embodiments, only some biomarkers are used in the methods described herein. For example, a single biomarker, 2 biomarkers, 3 biomarkers, 4 biomarkers, 5 biomarkers, 6 biomarkers, 7 biomarkers, 8 biomarkers, 9 biomarkers, 10 biomarkers, 11 biomarkers, 12 biomarkers, 13 biomarkers, 14 biomarkers, 15 biomarkers, 16 biomarkers, 17 biomarkers, 18 biomarkers, 19 biomarkers, or 20 biomarkers can be used in any combination. In other embodiments, all the biomarkers are used. The quantitative values may be combined in linear or non-linear fashion to calculate one or more risk scores for uveal melanoma for the individual, including further classifying uveal melanoma according to GEP class and/or PRAME status.

The level of a biomarker in a pre-treatment vitreous sample can be referred to as a “pre-treatment value” because the first vitreous sample is isolated from the individual prior to the administration of the therapy (i.e., “pre-treatment”). The level of a biomarker in the pre-treatment vitreous sample can also be referred to as a “baseline value” because this value is the value to which “post-treatment” values are compared. In some cases, the baseline value (i.e., “pre-treatment value”) is determined by determining the level of a biomarker in multiple (i.e., more than one, e.g., two or more, three or more, for or more, five or more, etc.) pre-treatment vitreous samples. In some cases, the multiple pre-treatment vitreous samples are isolated from an individual at different time points in order to assess natural fluctuations in biomarker levels prior to treatment. As such, in some cases, one or more (e.g., two or more, three or more, for or more, five or more, etc.) pre-treatment vitreous samples are isolated from the individual. In some embodiments, all of the pre-treatment vitreous samples will be the same type of vitreous sample (e.g., a biopsy sample). In some cases, two or more pre-treatment vitreous samples are pooled prior to determining the level of the biomarker in the vitreous samples. In some cases, the level of the biomarker is determined separately for two or more pre-treatment vitreous samples and a “pre-treatment value” is calculated by averaging the separate measurements.

A post-treatment vitreous sample is isolated from an individual after the administration of a therapy. Thus, the level of a biomarker in a post-treatment sample can be referred to as a “post-treatment value”. In some embodiments, the level of a biomarker is measured in additional post-treatment vitreous samples (e.g., a second, third, fourth, fifth, etc. post-treatment vitreous sample). Because additional post-treatment vitreous samples are isolated from the individual after the administration of a treatment, the levels of a biomarker in the additional vitreous samples can also be referred to as “post-treatment values.”

The term “responsive” as used herein means that the treatment is having the desired effect such as having anti-tumor activity. When the individual does not improve in response to the treatment, it may be desirable to seek a different therapy or treatment regime for the individual.

The determination that an individual has uveal melanoma and the classification of uveal melanoma by GEP class and/or PRAME status by expression profiling are active clinical applications of the correlation between levels of a biomarker and the disease. For example, “determining” requires the active step of reviewing the data, which is produced during the active assaying step(s), and resolving whether an individual does or does not have uveal melanoma. Additionally, in some cases, a decision is made to proceed with the current treatment (i.e., therapy), or instead to alter the treatment. In some cases, the subject methods include the step of continuing therapy or altering therapy.

The term “continue treatment” (i.e., continue therapy) is used herein to mean that the current course of treatment (e.g., continued administration of a therapy) is to continue. If the current course of treatment is not effective in treating uveal melanoma, the treatment may be altered. “Altering therapy” is used herein to mean “discontinuing therapy” or “changing the therapy” (e.g., changing the type of treatment, changing the particular dose and/or frequency of administration of medication, e.g., increasing the dose and/or frequency). In some cases, therapy can be altered until the individual is deemed to be responsive. In some embodiments, altering therapy means changing which type of treatment is administered, discontinuing a particular treatment altogether, etc.

As a non-limiting illustrative example, a patient may be initially treated for uveal melanoma by administering chemotherapy. Then to “continue treatment” would be to continue with this type of treatment. If the current course of treatment is not effective, the treatment may be altered, e.g., switching treatment to a different chemotherapy agent or increasing the dose or frequency of administration of the chemotherapy agent, or changing to a different type of treatment such as radiation therapy or surgery.

In other words, the level of one or more biomarkers may be monitored in order to determine when to continue therapy and/or when to alter therapy. As such, a post-treatment vitreous sample can be isolated after any of the administrations and the vitreous sample can be assayed to determine the level of a biomarker. Accordingly, the subject methods can be used to determine whether an individual being treated for uveal melanoma is responsive or is maintaining responsiveness to a treatment.

The therapy can be administered to an individual any time after a pre-treatment vitreous sample is isolated from the individual, but it is preferable for the therapy to be administered simultaneous with or as soon as possible (e.g., about 7 days or less, about 3 days or less, e.g., 2 days or less, 36 hours or less, 1 day or less, 20 hours or less, 18 hours or less, 12 hours or less, 9 hours or less, 6 hours or less, 3 hours or less, 2.5 hours or less, 2 hours or less, 1.5 hours or less, 1 hour or less, 45 minutes or less, 30 minutes or less, 20 minutes or less, 15 minutes or less, 10 minutes or less, 5 minutes or less, 2 minutes or less, or 1 minute or less) after a pre-treatment vitreous sample is isolated (or, when multiple pre-treatment vitreous samples are isolated, after the final pre-treatment vitreous sample is isolated).

In some cases, more than one type of therapy may be administered to the individual. For example, a subject who has uveal melanoma may be treated with a chemotherapeutic agent and surgery and/or radiation therapy. A subject diagnosed with GEP class 2 and/or PRAME positive uveal melanoma, who is at high risk of metastasis, may be treated more aggressively. For example, treatment of a high-risk patient may include, without limitation, adjuvant systemic therapy, radiation therapy, or surgery.

In some embodiments, the subject methods include providing an analysis indicating whether the individual is determined to have uveal melanoma. The analysis may further indicate whether the individual has GEP class 2 or PRAME positive uveal melanoma and is at high risk of metastasis (i.e., who should receive more aggressive treatment such as adjuvant systemic therapy, radiation therapy, or surgery). The analysis may further provide an analysis of whether an individual is responsive or not responsive to a treatment, or whether the individual is determined to be maintaining responsiveness or not maintaining responsiveness to a treatment for uveal melanoma. As described above, an analysis can be an oral or written report (e.g., written or electronic document). The analysis can be provided to the subject, to the subject's physician, to a testing facility, etc. The analysis can also be accessible as a website address via the internet. In some such cases, the analysis can be accessible by multiple different entities (e.g., the subject, the subject's physician, a testing facility, etc.).

### Detecting and Measuring Biomarkers

It is understood that the biomarkers in a sample can be measured by any suitable method known in the art. Measurement of the expression level of a biomarker can be direct or indirect. For example, the abundance levels of RNAs or proteins can be directly quantitated. Alternatively, the amount of a biomarker can be determined indirectly by measuring abundance levels of cDNAs, amplified RNAs or DNAs, or by measuring quantities or activities of RNAs, proteins, or other molecules (e.g., metabolites or metabolic byproducts) that are indicative of the expression level of the biomarker. The methods for measuring biomarkers in a sample have many applications. For example, one or more biomarkers can be measured to aid in diagnosing a patient with uveal melanoma and determining the appropriate treatment for a subject, as well as monitoring responses of a subject to treatment.

In some embodiments, the amount or level in the sample of one or more proteins/polypeptides encoded by a gene of interest is determined. Any convenient protocol for evaluating protein levels may be employed where the level of one or more proteins in the assayed sample is determined. For antibody-based methods of protein level determination, any convenient antibody can be used that specifically binds to the intended biomarker (e.g., FABP1, GM-CSF Ra, KLK7, SIGL6, MYC, OSM, SCFR/KIT, CSF2RB, c-MET/HGFR, SIR1, GRAA, MEF2C, ARGI1, FASLG, PP/PAHO, DNMT3A, DSG3, ENPP2, LEG9, and HGF and biomarkers listed in Tables 3, 4, 6, and 7). The terms “specifically binds” or “specific binding” as used herein refer to preferential binding to a molecule relative to other molecules or moieties in a solution or reaction mixture (e.g., an antibody specifically binds to a particular polypeptide or epitope relative to other available polypeptides or epitopes). In some embodiments, the affinity of one molecule for another molecule to which it specifically binds is characterized by a K_{d} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). By “affinity” it is meant the strength of binding, increased binding affinity being correlated with a lower K_{d}.

While a variety of different manners of assaying for protein levels are known in the art, one representative and convenient type of protocol for assaying protein levels is the enzyme-linked immunosorbent assay (ELISA). In ELISA and ELISA-based assays, one or more antibodies specific for the proteins of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Such conditions include diluting the sample with diluents such as BSA or bovine gamma globulin (BGG) in phosphate buffered saline (PBS)/Tween or PBS/Triton-X 100, which also tend to assist in the reduction of nonspecific background, and allowing the sample to incubate for about 2-4 hours at temperatures on the order of about 25°-27° C. (although other temperatures may be used). Following incubation, the antisera-contacted surface is washed so as to remove non-immunocomplexed material. An exemplary washing procedure includes washing with a solution such as PBS/Tween, PBS/Triton-X 100, or borate buffer. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g., urease, peroxidase, or alkaline phosphatase, which will generate a color precipitate upon incubating with an appropriate chromogenic substrate. For example, a urease or peroxidase-conjugated anti-human IgG may be employed, for a period of time and under conditions which favor the development of immunocomplex formation (e.g., incubation for 2 hours at room temperature in a PBS-containing solution such as PBS/Tween). After such incubation with the second antibody and washing to remove unbound material, the amount of label is quantified, for example by incubation with a chromogenic substrate such as urea and bromocresol purple in the case of a urease label or 2,2′-azino-di-(3-ethyl-benzthiazoline)-6-sulfonic acid (ABTS) and H₂O₂, in the case of a peroxidase label. Quantitation is then achieved by measuring the degree of color generation, e.g., using a visible spectrum spectrophotometer. The preceding format may be altered by first binding the sample to the assay plate. Then, primary antibody is incubated with the assay plate, followed by detecting of bound primary antibody using a labeled second antibody with specificity for the primary antibody.

The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected in a manner most appropriate for the substrate being used, for example, by removing a bead or dipstick from a reservoir, emptying or diluting a reservoir such as a microtiter plate well, or rinsing a bead, particle, chromatographic column or filter with a wash solution or solvent.

Alternatively, non-ELISA based-methods for measuring the levels of one or more proteins in a sample may be employed and any convenient method may be used. Representative examples known to one of ordinary skill in the art include but are not limited to other immunoassay techniques such as radioimmunoassays (RIA), sandwich immunoassays, fluorescent immunoassays, enzyme multiplied immunoassay technique (EMIT), capillary electrophoresis immunoassays (CEIA), and immunoprecipitation assays; mass spectrometry, or tandem mass spectrometry, proteomic arrays, xMAP microsphere technology, western blotting, immunohistochemistry, flow cytometry, cytometry by time-of-flight (CyTOF), multiplexed ion beam imaging (MIBI), and detection in body fluid by electrochemical sensor. In, for example, flow cytometry methods, the quantitative level of gene products of the one or more genes of interest are detected on cells in a cell suspension by lasers. As with ELISAs and immunohistochemistry, antibodies (e.g., monoclonal antibodies) that specifically bind the polypeptides encoded by the genes of interest are used in such methods.

As another example, electrochemical sensors may be employed. In such methods, a capture aptamer or an antibody that is specific for a target protein (the “analyte”) is immobilized on an electrode. A second aptamer or antibody, also specific for the target protein, is labeled with, for example, pyrroquinoline quinone glucose dehydrogenase ((PQQ)GDH). The sample of body fluid is introduced to the sensor either by submerging the electrodes in body fluid or by adding the sample fluid to a sample chamber, and the analyte allowed to interact with the labeled aptamer/antibody and the immobilized capture aptamer/antibody. Glucose is then provided to the sample, and the electric current generated by (PQQ)GDH is observed, where the amount of electric current passing through the electrochemical cell is directly related to the amount of analyte captured at the electrode.

For measuring protein activity levels, the amount or level of protein activity in the sample of one or more proteins/polypeptides encoded by the gene of interest is determined.

In other embodiments, the amount or level in the sample of one or more proteins is determined. Any convenient method for measuring protein levels in a sample may be used, e.g., antibody-based methods, e.g., immunoassays, e.g., enzyme-linked immunosorbent assays (ELISAs), immunohistochemistry, and mass spectrometry.

The resultant data provides information regarding expression, amount, and/or activity for each of the biomarkers that have been measured, wherein the information is in terms of whether or not the biomarker is present (e.g., expressed) and at what level, and wherein the data may be both qualitative and quantitative.

### Data Analysis

In some embodiments, one or more pattern recognition methods can be used in analyzing the data for biomarker levels. The quantitative values may be combined in linear or non-linear fashion to calculate one or more risk scores for uveal melanoma for an individual. In some embodiments, measurements for a biomarker or combinations of biomarkers are formulated into linear or non-linear models or algorithms (e.g., a ‘biomarker signature’) and converted into a likelihood score. This likelihood score indicates the probability that a vitreous sample is from a patient who may exhibit no evidence of disease, who may exhibit uveal melanoma. A likelihood score can also be used to distinguish among uveal melanoma disease subtypes, including classifying uveal melanoma by GEP class (i.e., GEP class 1 or class 2) and/or PRAME status (i.e., PRAME positive or negative). The models and/or algorithms can be provided in machine readable format, and may be used to correlate biomarker levels or a biomarker profile with a disease state, and/or to designate a treatment modality for a patient or class of patients.

Analyzing the levels of a plurality of biomarkers may comprise the use of an algorithm or classifier. In some embodiments, a machine learning algorithm is used to classify a patient as having uveal melanoma or further classify the patient by uveal melanoma subtype (e.g., GEP class 1, GEP class 2, PRAME positive, or PRAME negative uveal melanoma). The machine learning algorithm may comprise a supervised learning algorithm. Examples of supervised learning algorithms may include Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models.

The machine learning algorithms may also comprise an unsupervised learning algorithm. Examples of unsupervised learning algorithms may include artificial neural network, Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering.

In some instances, the machine learning algorithms comprise a reinforcement learning algorithm. Examples of reinforcement learning algorithms include, but are not limited to, temporal difference learning, Q-learning and Learning Automata. Alternatively, the machine learning algorithm may comprise Data Pre-processing.

Preferably, the machine learning algorithms may include, but are not limited to, Average One-Dependence Estimators (AODE), Fisher's linear discriminant, Logistic regression, Perceptron, Multilayer Perceptron, Artificial Neural Networks, Support vector machines, Quadratic classifiers, Boosting, Decision trees, C4.5, Bayesian networks, Hidden Markov models, High-Dimensional Discriminant Analysis, and Gaussian Mixture Models. The machine learning algorithm may comprise support vector machines, Naïve Bayes classifier, k-nearest neighbor, high-dimensional discriminant analysis, or Gaussian mixture models. In some instances, the machine learning algorithm comprises Random Forests.

### Kits

Also provided are kits for use in the methods. The subject kits include agents (e.g., an antibody that specifically binds to a biomarker and/or other immunoassay reagents, and the like) for determining the level of at least one biomarker. In some embodiments, a kit comprises agents for determining the level of a single biomarker, two or more different biomarkers, three or more different biomarkers, or all the biomarkers selected from the group consisting of FABP1, GM-CSF Ra, KLK7, SIGL6, MYC, OSM, SCFR/KIT, CSF2RB, c-MET/HGFR, SIR1, GRAA, MEF2C, ARGH1, FASLG, PP/PAHO, DNMT3A, DSG3, ENPP2, LEG9, and HGF biomarkers for diagnosing a patient with uveal melanoma. In some embodiments, the kit comprises agents for detecting one or more biomarkers selected from Table 3 and Table 6 for classifying uveal melanoma by GEP Class. In some embodiments, the kit comprises agents for detecting one or more biomarkers selected from Table 4 and Table 7 for classifying uveal melanoma by PRAME status.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, flash drive, and the like, on which the information has been recorded. Yet another form of these instructions that may be present is a website address which may be used via the internet to access the information at a removed site.

In certain embodiments, the kit further comprises reagents for performing an immunoassay. In some embodiments, the kit comprises an antibody that specifically binds to FABP1, an antibody that specifically binds to GM-CSF Ra, an antibody that specifically binds to KLK7, an antibody that specifically binds to SIGL6, an antibody that specifically binds to MYC, an antibody that specifically binds to OSM, an antibody that specifically binds to SCFR/KIT, an antibody that specifically binds to CSF2RB, an antibody that specifically binds to c-MET/HGFR, an antibody that specifically binds to SIR1, an antibody that specifically binds to GRAA, an antibody that specifically binds to MEF2C, an antibody that specifically binds to ARGH1, an antibody that specifically binds to FASLG, an antibody that specifically binds to PP/PAHO, an antibody that specifically binds to DNMT3A, an antibody that specifically binds to DSG3, an antibody that specifically binds to ENPP2, an antibody that specifically binds to LEG9, and an antibody that specifically binds to HGF.

In certain embodiments, the kit comprises agents for detecting oncostatin M (OSM), colony stimulating factor 2 common beta chain (CSF2RB), GM-CSF Ra, FABP1, kallikrein 7, oligodendrocyte-myelin glycoprotein (OMgp), sirtuin 1, siglec-6, myocyte-specific enhancer factor 2C (MEF2C), arginase-1, DNA (cytosine-5)-methyltransferase 3A (DNMT3A), and heparin-binding EGF-like growth factor (HB-EGF).

In certain embodiments, the kit comprises agents for detecting colony stimulating factor 2 common beta chain (CSF2RB), hepatocyte growth factor receptor (c-MET/HGFR), sirtuin-1, granzyme A, myocyte-specific enhancer factor 2C (MEF2C), arginase-1, Fas ligand (FASL), pancreatic prohormone (PP), and DNA (cytosine-5)-methyltransferase 3A (DNMT3A).

In certain embodiments, the kit comprises agents for detecting desmoglein-3, GM-CSF Ra, FABP1, kallikrein 7, and siglec-6.

In certain embodiments, the kit comprises agents for detecting desmoglein-3, autotaxin, galectin-9, hepatocyte growth factor (HGF), neogenin (NEO1), and pro-low-density lipoprotein receptor-related protein 1 (LRP1).

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-47 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method of diagnosing and treating uveal melanoma in a patient, the method comprising:
a) obtaining a vitreous sample from an eye of the patient;
b) measuring levels of expression of one or more biomarkers selected from the group consisting of fatty acid-binding protein 1 (FABP1), granulocyte-macrophage colony-stimulating factor receptor (GM-CSF Ra), kallikrein 7 (KLK7), sialic acid-binding Ig-like lectin 6 (SIGL6), Myc proto-oncogene protein (MYC), oncostatin-M (OSM), stem cell growth factor receptor Kit (SCFR/KIT), colony stimulating factor 2 common beta chain (CSF2RB), hepatocyte growth factor receptor (c-MET/HGFR), sirtuin-1 (SIR1), granzyme A (GRAA), myocyte-specific enhancer factor 2C (MEF2C), arginase-1 (ARGH1), fas ligand (FASLG), pancreatic prohormone (PP/PAHO), DNA (cytosine-5)-methyltransferase 3A (DNMT3A), desmoglein-3 (DSG3), autotaxin (ENPP2), galectin-9 (LEG9), and hepatocyte growth factor (HGF) in the vitreous sample, wherein differential expression of FABP1, GM-CSF Ra, KLK7, SIGL6, MYC, OSM, SCFR/KIT, CSF2RB, c-MET/HGFR, SIR1, GRAA, MEF2C, ARGH1, FASLG, PP/PAHO, DNMT3A, DSG3, ENPP2, LEG9, and HGF compared to reference value ranges for a vitreous sample from a control subject indicate that the patient has uveal melanoma; and
c) treating the patient for the uveal melanoma, if the patient has a positive diagnosis for said uveal melanoma based on the levels of expression of the one or more biomarkers.

2. The method of aspect 2, wherein increased levels of expression of SIGL6, c-MYC, OSM, and SCFR/c-Kit and decreased levels of expression of FABP1, GM-CSF Ra, and KLK7 compared to reference value ranges for the biomarkers in a vitreous sample from a control subject indicate that the patient has uveal melanoma.

3. The method of any one of aspects 1 to 3, further comprising classifying the uveal melanoma by gene expression profile (GEP) class if the patient has a positive diagnosis for uveal melanoma by comparing the levels of expression of one or more biomarkers selected from Table 3 and Table 6 in the vitreous sample from the eye of the patient to reference value ranges for the one or more biomarkers obtained from one or more reference vitreous samples from one or more reference subjects having uveal melanoma that has been classified by gene expression profile (GEP) class.

4. The method of aspect 3, wherein the one or more biomarkers are selected from the group consisting of oncostatin M (OSM), colony stimulating factor 2 common beta chain (CSF2RB), GM-CSF Ra, FABP1, kallikrein 7, oligodendrocyte-myelin glycoprotein (OMgp), sirtuin 1, siglec-6, myocyte-specific enhancer factor 2C (MEF2C), arginase-1, DNA (cytosine-5)-methyltransferase 3A (DNMT3A), and heparin-binding EGF-like growth factor (HB-EGF).

5. The method of aspect 4, wherein the one or more biomarkers comprise or consist of oncostatin M (OSM), colony stimulating factor 2 common beta chain (CSF2RB), GM-CSF Ra, FABP1, kallikrein 7, oligodendrocyte-myelin glycoprotein (OMgp), sirtuin 1, siglec-6, myocyte-specific enhancer factor 2C (MEF2C), arginase-1, DNA (cytosine-5)-methyltransferase 3A (DNMT3A), and heparin-binding EGF-like growth factor (HB-EGF).

6. The method of aspect 3, wherein increased levels of expression of colony stimulating factor 2 common beta chain (CSF2RB), hepatocyte growth factor receptor (c-MET/HGFR), sirtuin-1, granzyme A, myocyte-specific enhancer factor 2C (MEF2C), arginase-1, Fas ligand (FASL), pancreatic prohormone (PP), and DNA (cytosine-5)-methyltransferase 3A (DNMT3A) compared to reference value ranges for a vitreous sample from a control subject indicate that the patient has GEP Class 2 uveal melanoma.

7. The method of any one of aspects 1 to 6, further comprising classifying the uveal melanoma by PRAME status if the patient has a positive diagnosis for uveal melanoma by comparing the levels of expression of one or more biomarkers selected from Table 4 and Table 7 in the vitreous sample from the eye of the patient to reference value ranges for the one or more biomarkers obtained from one or more reference vitreous samples from one or more reference subjects having uveal melanoma that has been classified by PRAME status.

8. The method of aspect 7, wherein the one or more biomarkers are selected from the group consisting of desmoglein-3, GM-CSF Ra, FABP1, kallikrein 7, and siglec-6.

9. The method of aspect 8, wherein the one or more biomarkers comprise or consist of desmoglein-3, GM-CSF Ra, FABP1, kallikrein 7, and siglec-6.

10. The method of aspect 7, wherein increased levels of expression of desmoglein-3, autotaxin, galectin-9, hepatocyte growth factor (HGF), neogenin (NEO1), and pro-low-density lipoprotein receptor-related protein 1 (LRP1) indicate that the patient has PRAME positive uveal melanoma.

11. The method of any one of aspects 1 to 10, wherein the patient has been diagnosed with idiopathic uveitis.

12. The method of any one of aspects 1 to 11, further comprising detecting leukocytes in the vitreous humor or active chorioretinal inflammation in the patient.

13. The method of any one of aspects 1 to 12, wherein said treating the patient for uveal melanoma comprises administering adjuvant systemic therapy, radioactive plaque therapy, external beam proton therapy, laser therapy, enucleation, evisceration, exenteration, iridectomy, choroidectomy, iridocyclectomy, eyewall resection, chemotherapy, brachytherapy, transpupillary thermotherapy, resection of the eye tumor, gamma knife stereotactic radiosurgery, or a combination thereof.

14. The method of any one of aspects 1 to 13, wherein said measuring the levels of expression comprises performing mass spectrometry, tandem mass spectrometry, liquid chromatography, liquid chromatography-tandem mass spectrometry (LC-MS/MS), NMR, an enzyme-linked immunosorbent assay (ELISA), a radioimmunoassay (RIA), an immunofluorescent assay (IFA), immunohistochemistry, fluorescence-activated cell sorting (FACS), or a Western Blot.

15. The method of aspect 14, wherein the ELISA is performed using a multiplex ELISA array.

16. The method of any one of aspects 1 to 15, further comprising performing ultrasonography, fluorescein angiography, optical coherence tomography, autofluorescence, indocyanine green angiography, or a radioactive phosphorus uptake test on the eye.

17. The method of any one of aspects 1 to 16, further comprising genotyping the patient to determine if the patient has one or more chromosomal abnormalities or mutations linked to uveal melanoma.

18. The method of aspect 17, wherein the one or more chromosomal abnormalities are selected from the group consisting of monosomy 3 (M3), gain of long arm of chromosome 8 (8q+), deletion of chromosome 1p (1p−), and changes within chromosome 6 (6p+ or 6q−).

19. A method of subtyping uveal melanoma and determining risk of metastasis, the method comprising:
a) obtaining a vitreous sample from an eye of a patient who has uveal melanoma;
b) measuring levels of expression of one or more biomarkers selected from the group consisting of colony stimulating factor 2 common beta chain (CSF2RB), hepatocyte growth factor receptor (c-MET/HGFR), sirtuin-1, granzyme A, myocyte-specific enhancer factor 2C (MEF2C), arginase-1, Fas ligand (FASL), pancreatic prohormone (PP), and DNA (cytosine-5)-methyltransferase 3A (DNMT3A), wherein increased levels of expression of the one or more biomarkers selected from the group consisting of CSF2RB, c-MET/HGFR, sirtuin-1, granzyme A, MEF2C, arginase-1, FASL, PP, DNMT3A in the vitreous sample from the patient compared to reference value ranges for the biomarkers from a vitreous sample from a control subject indicate that the patient has GEP class 2 uveal melanoma and is at risk of metastasis; and
c) measuring levels of expression of one or more biomarkers selected from the group consisting of desmoglein-3, autotaxin, galectin-9, hepatocyte growth factor (HGF), neogenin (NEO1), and pro-low-density lipoprotein receptor-related protein 1 (LRP1) wherein increased levels of expression of the one or more biomarkers selected from the group consisting of desmoglein-3, autotaxin, galectin-9, HGF, NEO1, and LRP1 in the vitreous sample from the eye of the patient compared to reference value ranges for the biomarkers from a vitreous sample from a control subject indicate that the patient has PRAME positive uveal melanoma and is at risk of metastasis.

20. The method of aspect 19, further comprising administering adjuvant systemic therapy, radiotherapy, or performing surgery if the patient is diagnosed with GEP class 2 uveal melanoma or PRAME positive uveal melanoma.

21. The method of aspect 19 or 20, further comprising measuring levels of one or more additional biomarkers selected from Table 3, Table 4, Table 6, and Table 7.

22. A method of monitoring uveal melanoma in a patient, the method comprising:
a) obtaining a first vitreous sample from an eye of the patient at a first time point and a second vitreous sample from the eye of the subject later at a second time point;
b) measuring one or more biomarkers in the first vitreous sample and the second vitreous sample, wherein the biomarkers are selected from the group consisting of SIGL6, c-MYC, OSM, SCFR/c-Kit, FABP1, KLK7, GM-CSF Ra, and serpin 1; and
c) analyzing the levels of expression of the one or more biomarkers in conjunction with respective reference value ranges for said biomarkers, wherein detection of increased levels of expression of SIGL6, c-MYC, OSM, and SCFR/c-Kit and decreased levels of expression of FABP1, KLK7, GM-CSF Ra, and serpin 1 in the second vitreous sample compared to the first vitreous sample indicate that the patient is worsening, and detection of decreased levels of expression of SIGL6, c-MYC, OSM, and SCFR/c-Kit and increased levels of expression of FABP1, KLK7, GM-CSF Ra, and serpin 1 in the second vitreous sample compared to the first vitreous sample indicate that the patient is improving.

23. A method of monitoring efficacy of a treatment of a patient for uveal melanoma, the method comprising:
a) obtaining a first vitreous sample from the patient before the patient undergoes the treatment and a second vitreous sample from the subject after the patient undergoes the treatment;
b) measuring one or more biomarkers in the first vitreous sample and the second vitreous sample, wherein the biomarkers are selected from the group consisting of SIGL6, c-MYC, OSM, SCFR/c-Kit, FABP1, KLK7, GM-CSF Ra, and serpin 1; and
c) evaluating the efficacy of the treatment, wherein detection of increased levels of expression of SIGL6, c-MYC, OSM, and SCFR/c-Kit and decreased levels of expression of FABP1, KLK7, GM-CSF Ra, and serpin 1 in the second vitreous sample compared to the first vitreous sample indicate that the patient is worsening or not responding to the treatment, and detection of decreased levels of expression of SIGL6, c-MYC, OSM, and SCFR/c-Kit and increased levels of expression of FABP1, KLK7, GM-CSF Ra, and serpin 1 in the second vitreous sample compared to the first vitreous sample indicate that the patient is improving.

24. The method of aspect 23, further comprising altering the treatment if the patient is worsening or not responding to the treatment.

25. A kit comprising agents for detecting at least 3 biomarkers selected from the group consisting of fatty acid-binding protein 1 (FABP1), granulocyte-macrophage colony-stimulating factor receptor (GM-CSF Ra), kallikrein 7 (KLK7), sialic acid-binding Ig-like lectin 6 (SIGL6), Myc proto-oncogene protein (MYC), oncostatin-M (OSM), stem cell growth factor receptor Kit (SCFR/KIT), common beta chain (CSF2RB), hepatocyte growth factor receptor (c-MET/HGFR), sirtuin-1 (SIR1), granzyme A (GRAA), myocyte-specific enhancer factor 2C (MEF2C), arginase-1 (ARGH1), fas ligand (FASLG), pancreatic prohormone (PP/PAHO), DNA (cytosine-5)-methyltransferase 3A (DNMT3A), desmoglein-3 (DSG3), autotaxin (ENPP2), galectin-9 (LEG9), and hepatocyte growth factor (HGF).

26. The kit of aspect 25, wherein the kit comprises agents for detecting the FABP1, GM-CSF Ra, KLK7, SIGL6, MYC, OSM, SCFR/KIT, CSF2RB, c-MET/HGFR, SIR1, GRAA, MEF2C, ARGH1, FASLG, PP/PAHO, DNMT3A, DSG3, ENPP2, LEG9, and HGF biomarkers.

27. The kit of aspect 25 or 26, wherein the kit comprises agents for detecting the SIGL6, c-MYC, OSM, SCFR/c-Kit, FABP1, GM-CSF Ra, and KLK7 biomarkers.

28. The kit of any one of aspects 25 to 27, further comprising agents for detecting one or more biomarkers selected from Table 3, Table 4, Table 6, or Table 7.

29. The kit of aspect 28, wherein the kit comprises agents for detecting oncostatin M (OSM), colony stimulating factor 2 common beta chain (CSF2RB), GM-CSF Ra, FABP1, kallikrein 7, oligodendrocyte-myelin glycoprotein (OMgp), sirtuin 1, siglec-6, myocyte-specific enhancer factor 2C (MEF2C), arginase-1, DNA (cytosine-5)-methyltransferase 3A (DNMT3A), and heparin-binding EGF-like growth factor (HB-EGF).

30. The kit of aspect 28, wherein the kit comprises agents for detecting colony stimulating factor 2 common beta chain (CSF2RB), hepatocyte growth factor receptor (c-MET/HGFR), sirtuin-1, granzyme A, myocyte-specific enhancer factor 2C (MEF2C), arginase-1, Fas ligand (FASL), pancreatic prohormone (PP), and DNA (cytosine-5)-methyltransferase 3A (DNMT3A).

31. The kit of aspect 28, wherein the kit comprises agents for detecting desmoglein-3, GM-CSF Ra, FABP1, kallikrein 7, and siglec-6.

32. The kit of aspect 28, wherein the kit comprises agents for detecting desmoglein-3, autotaxin, galectin-9, hepatocyte growth factor (HGF), neogenin (NEO1), and pro-low-density lipoprotein receptor-related protein 1 (LRP1).

33. The kit of any one of aspects 25 to 32, further comprising reagents for performing an immunoassay.

34. The kit of any one of aspects 25 to 33, further comprising instructions for diagnosing uveal melanoma.

35. The kit of any one of aspects 25 to 34, further comprising an antibody that specifically binds to FABP1, an antibody that specifically binds to GM-CSF Ra, an antibody that specifically binds to KLK7, an antibody that specifically binds to SIGL6, an antibody that specifically binds to MYC, an antibody that specifically binds to OSM, an antibody that specifically binds to SCFR/KIT, an antibody that specifically binds to CSF2RB, an antibody that specifically binds to c-MET/HGFR, an antibody that specifically binds to SIR1, an antibody that specifically binds to GRAA, an antibody that specifically binds to MEF2C, an antibody that specifically binds to ARGH1, an antibody that specifically binds to FASLG, an antibody that specifically binds to PP/PAHO, an antibody that specifically binds to DNMT3A, an antibody that specifically binds to DSG3, an antibody that specifically binds to ENPP2, an antibody that specifically binds to LEG9, and an antibody that specifically binds to HGF.

36. A protein selected from the group consisting of fatty acid-binding protein 1 (FABP1), granulocyte-macrophage colony-stimulating factor receptor (GM-CSF Ra), kallikrein 7 (KLK7), sialic acid-binding Ig-like lectin 6 (SIGL6), Myc proto-oncogene protein (MYC), oncostatin-M (OSM), stem cell growth factor receptor Kit (SCFR/KIT), common beta chain (CSF2RB), hepatocyte growth factor receptor (c-MET/HGFR), sirtuin-1 (SIR1), granzyme A (GRAA), myocyte-specific enhancer factor 2C (MEF2C), arginase-1 (ARGH1), fas ligand (FASLG), pancreatic prohormone (PP/PAHO), DNA (cytosine-5)-methyltransferase 3A (DNMT3A), desmoglein-3 (DSG3), autotaxin (ENPP2), galectin-9 (LEG9), and hepatocyte growth factor (HGF) for use as a biomarker in diagnosing uveal melanoma.

37. A protein selected from the group consisting of colony stimulating factor 2 common beta chain (CSF2RB), hepatocyte growth factor receptor (c-MET/HGFR), sirtuin-1, granzyme A, myocyte-specific enhancer factor 2C (MEF2C), arginase-1, Fas ligand (FASL), pancreatic prohormone (PP), and DNA (cytosine-5)-methyltransferase 3A (DNMT3A) for use as a biomarker in diagnosing GEP Class 2 uveal melanoma.

38. A protein selected from the group consisting of desmoglein-3, autotaxin, galectin-9, hepatocyte growth factor (HGF), neogenin (NEO1), and pro-low-density lipoprotein receptor-related protein 1 (LRP1) for use as a biomarker in diagnosing PRAME positive uveal melanoma.

39. An in vitro method of diagnosing uveal melanoma, the method comprising:
a) obtaining a vitreous sample from an eye of the patient; and
b) measuring levels of expression of at least 3 biomarkers selected from the group consisting of fatty acid-binding protein 1 (FABP1), granulocyte-macrophage colony-stimulating factor receptor (GM-CSF Ra), kallikrein 7 (KLK7), sialic acid-binding Ig-like lectin 6 (SIGL6), Myc proto-oncogene protein (MYC), oncostatin-M (OSM), stem cell growth factor receptor Kit (SCFR/KIT), common beta chain (CSF2RB), hepatocyte growth factor receptor (c-MET/HGFR), sirtuin-1 (SIR1), granzyme A (GRAA), myocyte-specific enhancer factor 2C (MEF2C), arginase-1 (ARGI1), fas ligand (FASLG), pancreatic prohormone (PP/PAHO), DNA (cytosine-5)-methyltransferase 3A (DNMT3A), desmoglein-3 (DSG3), autotaxin (ENPP2), galectin-9 (LEG9), and hepatocyte growth factor (HGF) in the vitreous sample, wherein differential expression of the FABP1, GM-CSF Ra, KLK7, SIGL6, MYC, OSM, SCFR/KIT, CSF2RB, c-MET/HGFR, SIR1, GRAA, MEF2C, ARGI1, FASLG, PP/PAHO, DNMT3A, DSG3, ENPP2, LEG9, and HGF compared to reference value ranges for a control sample indicate that the patient has the uveal melanoma.

It will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

Proteomic Analysis of Uveal Melanoma Reveals Candidate Biomarkers Associated with Increased Metastatic Risk

### Introduction

Proteomic analysis is becoming an attractive and powerful tool for characterizing the molecular profiles of diseased tissues [18]. The proteome of vitreous (intraocular fluid) in patients with UM can be characterized to uncover candidate biomarkers for metastasis and cancer progression that can later be validated in serum. To advance precision health approaches for uveal melanoma, our group has created a novel device and software that allows for immediate and point-of-care processing of liquid biopsy specimens. Our group has used large-scale proteomic platforms to analyze the protein signature in vitreous biopsies from patients with many vitreoretinal diseases. This approach allowed us to identify a “short list’ of several secreted candidate biomarkers that can reliably differentiate UM from control vitreous. It is the hope that by earlier identification of UM will influence the schedule of treatment and increase survival.

### Case Series

Case 1—A 67-year-old male presented for consultation for a uveal melanoma in the left eye measuring 16 mm×13.66 mm×5.8 mm. The patient reported a six-month history of blurry vision with waves of light. He was seen by a local eye doctor who noted an ocular lesion and retinal detachment prior to referral. He had no prior history of eye surgery or eye problems. Left eye enucleation with orbital implant was subsequently performed and a biopsy was sent for genetic testing. The biopsy revealed both spindle and epithelioid cells (G2) with focal emissary vein and inner scleral invasion without extraocular tumor extension (EOE). The tumor was found to be gene expression profile (GEP) Class 1B, or intermediate risk disomy 3, and PRAME positive [19-21]. The American Joint Committee on Cancer (AJCC) classification was T3a with an anatomic stage Ill (T3, N0, M0) [22, 23].

Case 2—A 30-year-old female with a family history of cancer (BAP1 deletion) presented for a second opinion regarding a retinal lesion in the left eye found to be uveal melanoma measuring 6 mm×7 mm×2.5 mm. The patient had regular eye exams for the past 4 years but, by her account, had never been dilated at these exams. She noted a two-month history of worsening vision of the left eye. She was seen by an optometrist and subsequently a retina specialist and found to have a peripapillary elevated lesion of the left eye. She has a strong family history of cancer, including mesothelioma in her grandmother, and breast and lung cancer in her mother. She had a breast and thyroid thermography test performed which showed some suspicious activity in her thyroid but no concerning breast activity. Left eye enucleation with orbital implant was subsequently performed and a biopsy was sent for genetic testing. The biopsy revealed both spindle A and spindle B cells (G1). The tumor was found to be GEP Class 1A, or low risk disomy 3, PRAME positive. The AJCC classification was T1a with an anatomic stage I (T1a, N0, M0).

Case 3—A 80-year-old male with a history of parkinsonism and ANS dysfunction presented for a consultation for retinal detachment in the left eye and was found to have uveal melanoma measuring 12.7 mm×11.22 mm×4.75 mm. The patient had undergone cataract surgery in both eyes six months prior and had blurry vision in the left eye since that time. His vision had been stably poor with no flashes or floaters and very mild intermittent pain. Left eye enucleation with orbital implant was performed and a biopsy was sent for genetic testing. The biopsy revealed >90% epithelioid cells (G3). The tumor was found to be GEP Class 2, PRAME positive. The AJCC classification was T2a with an anatomic stage IIA (T2a, N0, M0).

Case 4—A 47-year-old male presented for a consultation for a mass in the right eye found to be an amelanotic uveal melanoma measuring 11.48 mm×11.75 mm×7.5 mm. The patient described a subtle decrease in vision in the right eye three weeks prior to presentation with a curtain veil noted. He had been seen 9-months prior by the referring physician with a normal posterior segment exam. Right eye I-125 plaque brachytherapy was performed with a vitrectomy assisted-FNA biopsy and injection of bevacizumab at the time of plaque insertion. The biopsy revealed predominantly epithelioid cells (G3) and the SOX10 immunohistochemistry stain was positive in lesional cells. The tumor was found to be GEP Class 1A, PRAME negative. The AJCC classification was T2a with an anatomic stage IIA (T2a, NX, MX).

Case 5—A 57-year-old diabetic male presented on for a consultation for ocular melanoma in the right eye measuring 11 mm×9 mm×3.99 mm. The patient had no vision changes and had initially been sent for a diabetic eye exam when a choroidal lesion was noted in the right eye. Right eye I-125 plaque brachytherapy was performed with a vitrectomy assisted-FNA biopsy at the time of insertion. The tumor was found to be GEP Class 1A, PRAME negative. The pathology did not result. The AJCC classification was T2a with an anatomic stage IIA (T2a, NX, MX).

Case 6—A 57-year-old male presented for a consultation for ciliary body mass in the left eye found to be uveal melanoma measuring 21.8 mm×16.66 mm×12.74 mm. The patient noted a two-year history of vision changes which worsened over the past 2 months with no other prior vision problems. He had been trying unsuccessfully for 1-2 months to obtain a functional prescription for eyeglass. He denied pain but noted a pressure-like sensation. He reported darkness in his peripheral field of vision as well as photopsias for the past 3 weeks with dramatic changes leading up to his presentation. The patient's last dilated fundus examination had been performed one year prior to the noticed growth in his eyes by his doctor. Following this, he was seen by two referring physicians who reported large ciliary body mass lesions. Ophthalmic imaging showed the right eye with flat dark choroidal nevus and left eye with large temporal mass with inferior retinal detachment. Left eye enucleation with orbital implant was performed and a biopsy was sent for genetic testing. The biopsy revealed mixed spindle and epithelioid cell types (G2) confined to the uveal tract and abutting the ciliary body with negative surgical margins. The tumor was found to be GEP Class 2, PRAME positive. There were no metastases noted at the time of the procedure, but liver metastases have since been noted. The AJCC classification was T4b with an anatomic stage IIB (T3a, N0, M0) at the time of procedure and IV (T4b, N1, M1) following.

Case 7—A 35-year-old female with a history of Henoch-Schönlein purpura presented for a consultation for possible subretinal fluid in left eye and was found to have uveal melanoma measuring 15.6 mm×15.9 mm×7.3 mm. The patient noted several years of an intermittent “heat wave” pattern in the left eye with a 1-month history of peripheral vision changes. She experienced rare floaters but no photopsias, eye pain, photophobia nor trauma. She denied prior eye surgery, laser procedures, or intravitreal injections. Left eye I-125 plaque brachytherapy was performed with a vitrectomy assisted-FNA biopsy at the time of insertion. The tumor was found to be GEP Class 1B, PRAME negative. The pathology did not result. The AJCC classification was T3a with an anatomic stage IIB (T3a, N0, M0).

Case 8—A 58-year-old male with a recent diagnosis of prostate cancer (the day prior to presentation) presented for a consultation for choroidal lesion to the left eye found to be uveal melanoma measuring 15.6 mm×14.5 mm×7.3 mm. The patient awoke two weeks prior and noticed that vision in the left eye was significantly decreased with an increase in floaters. He denied recent trauma or eye pain. He immediately saw an optometrist who noted “bleeding in the eye” and suspected a retinal detachment possibly with a growth noted. The patient saw another referring patient on the same day who performed an ultrasound which revealed possible melanoma with a vitreous hemorrhage. He described his vision in the left eye as looking through fog and shadow with an inability to read or focus. The patient's mother has glaucoma and his older brother was previously diagnosed with prostate cancer as well. Right eye I-125 plaque brachytherapy was performed with a vitrectomy assisted-FNA biopsy and injection of bevacizumab at the time of plaque insertion. Biopsy revealed a primarily epithelioid morphology (G3). The tumor was found to be GEP Class 2, PRAME negative. The AJCC classification was T3a with an anatomic stage IIB (T3a, N0, M0).

### Results

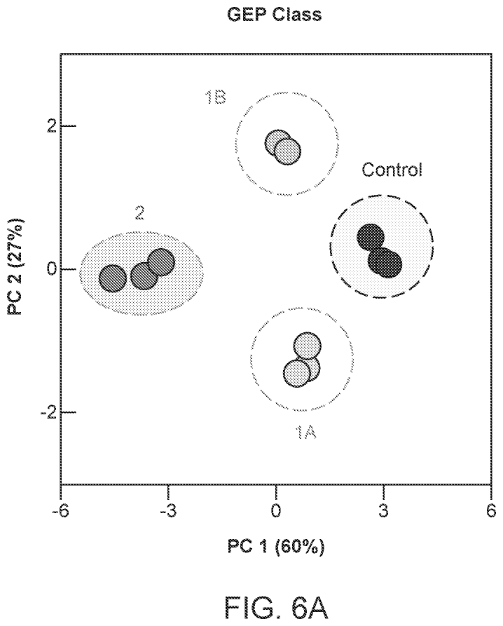

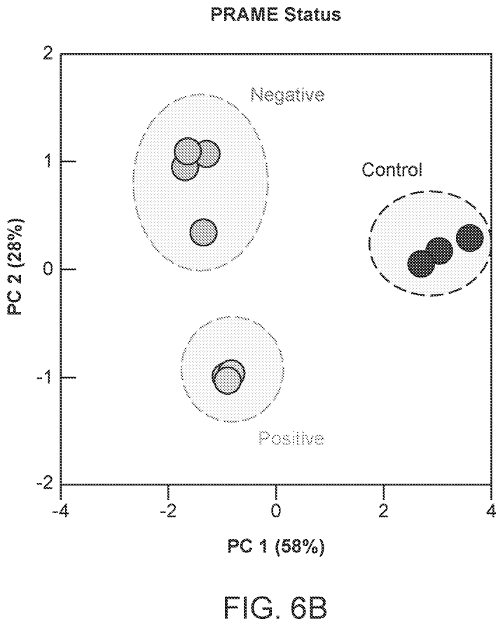

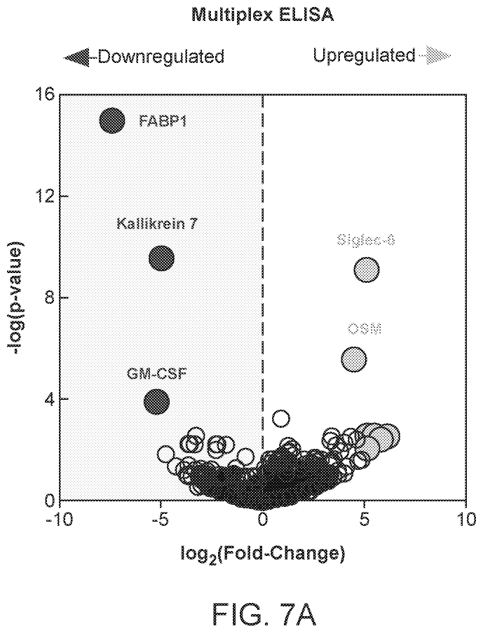

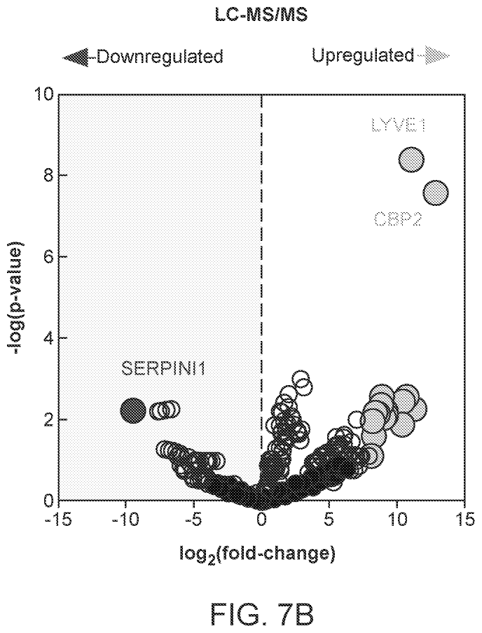

Targeted proteomic analysis distinguishes molecular classes of uveal melanoma—The current prognostic classification of uveal melanoma tumors is performed by direct molecular genetic testing of the primary tumor. While highly informative, these prognostic biopsies are invasive and carry the risk of extraocular extension of the tumor [24-26]. We therefore sought to identify vitreous proteins that could serve as accessible biomarkers in place of tumor biopsies. To identify candidate protein biomarkers, liquid biopsies from patient eyes were screened using a targeted proteomic platform that can quantitatively measure 1,000 cytokine-signaling molecules simultaneously—a precision medicine strategy. Biopsies were analyzed using this membrane-based antibody array to identify any abnormally expressed proteins and determine a protein signature. Protein concentrations were first analyzed by principal component analysis (PCA). Multi-group comparison (1-way ANOVA) followed by Benjamini and Hochberg false-discovery rate (FDR) corrections was used to identify differentially expressed proteins in the large-scale dataset. The score plot of PC1 and PC2 showed separation between the 8 uveal melanoma (UM) cases and 3 controls based on differentially expressed proteins that were significantly different between the two groups (FDR=0.0004%, adjusted p-value=5.4e-4; FIG. 6). A total of 77 proteins were differentially expressed among control and UM samples (62 upregulated proteins and 15 downregulated proteins; p<0.05; FIG. 7A).

Among the proteins upregulated in UM vitreous were Sialic acid immunoglobulin-like lectin 6 (Siglec-6), c-MYC, Oncostatin M (OSM), and Stem cell factor receptor (SCFR/c-Kit). Siglec-6 is a member of the immunoglobulin superfamily which binds sialic acid [27]. Following malignant transformation, cancer cells can overexpress sialic acid which elicits an immune response through Siglec interactions. Significant increase in Siglec-6 levels have been shown to occur in situ with colon cancer cells and under hypoxic conditions commonly found in tumor microenvironments, implicating Siglec-6 as a functionally inhibitory receptor [28]. OSM is a cytokine belonging to the gp130 family and plays a complex role in cancer biology. It was originally shown to inhibit proliferation of a number of cancer cell lines in vitro, including melanoma, osteosarcoma, and breast cancer. However, a pro-tumorigenic role for OSM in other types of cancers was later described [29]. Furthermore, OSM has been shown to promote cancer cell plasticity through STAT3-SMAD3 signaling pathways [30]. Thus, the precise role of OSM depends heavily on its context. The role of c-MYC in cancer cellular growth and metabolism has been extensively studied, particularly in the context of Burkitt lymphoma [31]. Cancer cells overexpress c-MYC, which results in increased mitochondrial biogenesis, glycolysis, and rRNA and protein biosynthesis [31]. SCFR/c-Kit promotes cancer cell proliferation by stimulating mTOR/PI3K and ERK signaling pathways [32, 33].

There were several proteins that were highly expressed in control patients and not present in UM vitreous: Fatty acid binding protein 1 (FABP1), Granulocyte-macrophage colony-stimulating factor (GM-CSF Ra), and Kallikrein 7 (KLK7). These results gave us confidence that we could identify vitreous protein signatures that distinguish uveal melanoma from controls. Liver fatty acid-binding protein (FABP1) is involved in the binding of long chain fatty acids and is expressed in the intestine, liver, pancreas, stomach and kidney [34]. FABP1 is thought to play a significant role in liver diseases including hepatocellular carcinoma (HCC) [35-38]. Overexpression of FABP1 has been found in hepatocellular carcinoma, lung, and colorectal cancers. It has been shown to promote tumor growth and metastasis in mouse models of HCC through upregulation of VEGFA and an increase in migration activity [39]. GM-CSF is known both for its role as an immunomodulator [40] and has been found to inhibit the proliferation of small-cell lung cancer by blocking cell cycle progression [41, 42]. GM-CSF has already been used in the treatment of cancer in several different modalities. In a Phase II trial for metastatic cutaneous melanoma, combined Ipilimumab and GM-CSF was safe and more effective than Ipilimumab monotherapy [43]. KLK7 is a member of a class of serine proteases involved in extracellular matrix remodeling, skin desquamation, processing of hormone precursors, and regulation of tumor cell proliferation. KLK7 performs important roles in the skin and in cancer, where it functions to degrade the extracellular matrix and cellular adhesion molecules.

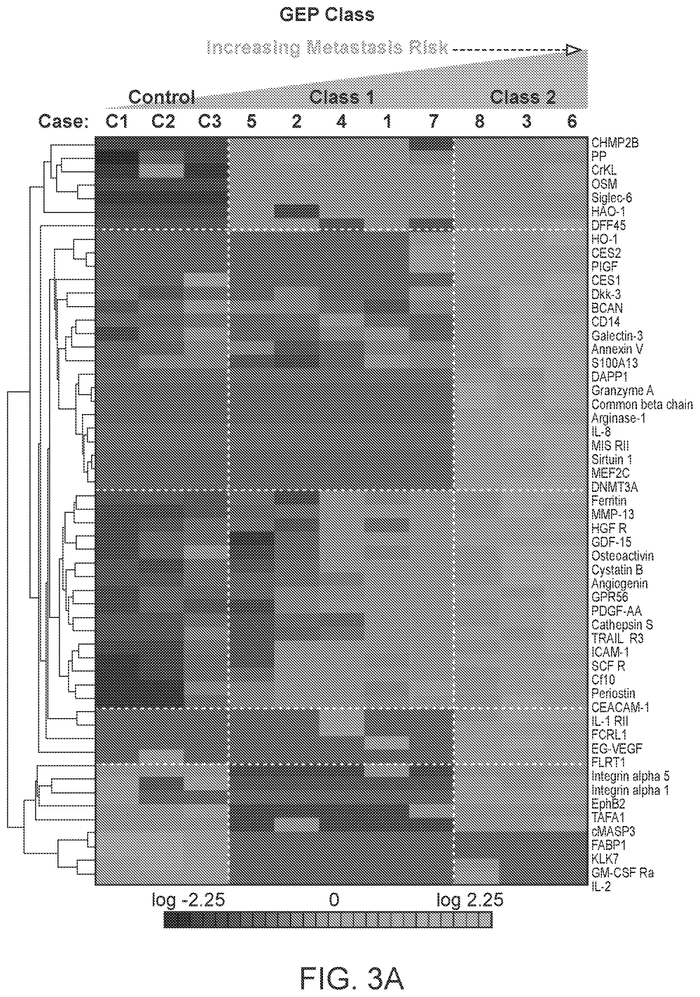

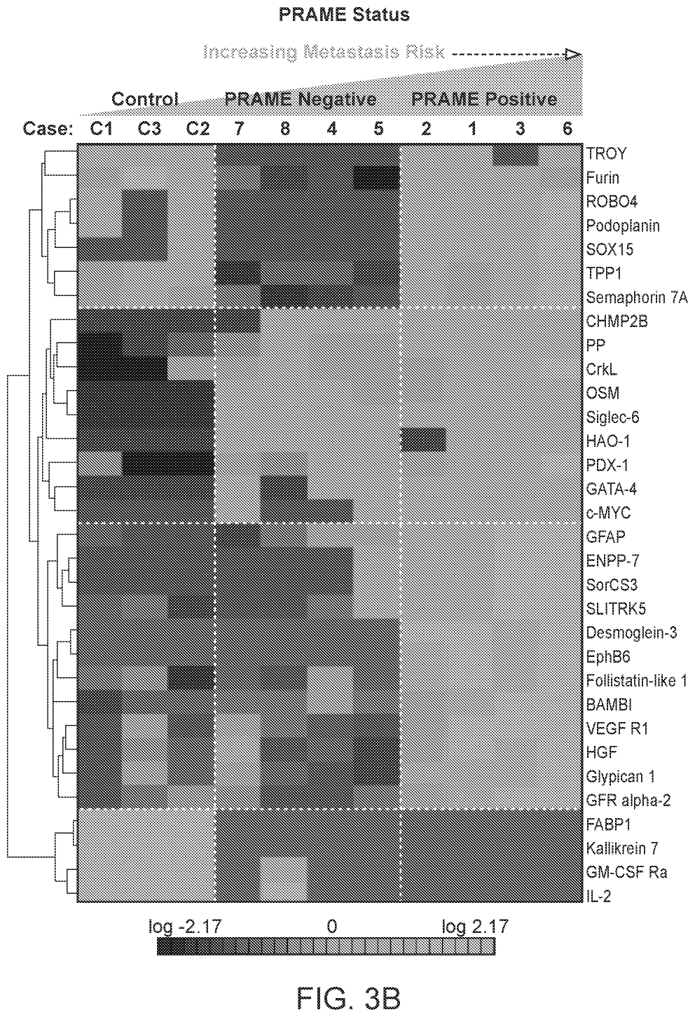

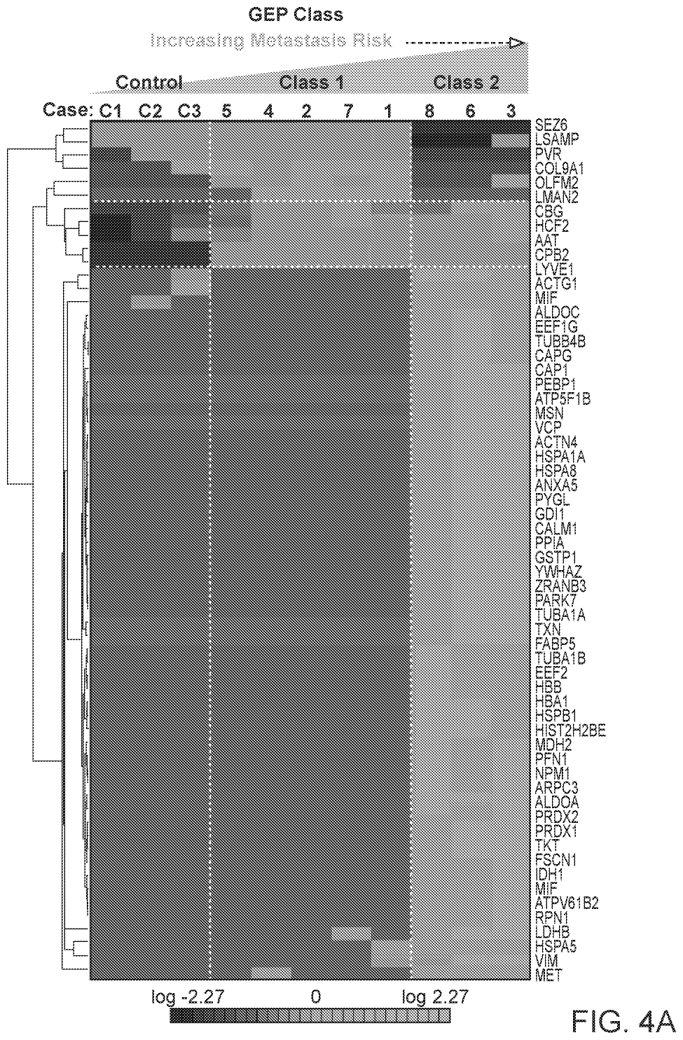

Since primary UM tumors can be classified by gene expression profile (GEP class) or PRAME status, we sought to identify protein signatures that were associated with these molecular classifications. When comparing protein expression by GEP class, there were 46 differentially expressed proteins at the p<0.01 level (FIG. 3A; Table 3). Among the upregulated proteins in GEP Class 2 patients were: Hepatocyte growth factor receptor (HGFR/c-MET), Sirtuin-1 (SIRT1), Pancreatic prohormone (PP), DNA (cytosine-5)-methyltransferase 3A (DNMT3A), Myocyte-specific enhancer factor 2C (MEF2C), Colony stimulating factor 2 common β chain (βc), Arginase-1, Granzyme A, and Fas ligand (FASL). DNMT3A is frequently mutated in cancer. It is one of the 127 frequently mutated genes identified in the Cancer Genome Atlas project (TCGA) and overexpression is associated with poor prognosis in human HCC [44, 45]. MEF2C is required for normal neural differentiation, but also hematopoietic differentiation towards lymphoid rather than myeloid lineages. This is consistent with the increased expression of CD4 seen in UM vitreous. βc regulates the differentiation of granulocytes and macrophages [46]. Arginase-1 converts L-arginine to L-ornithine and urea and is an immunohistochemical marker for HCC [47]. Cancer cells have been demonstrated to overexpress Arginase-1 to deplete effector T-cells of L-arginine, leading to impaired T-cell function, allowing tumors to evade the host immune system [48]. Pancreatic prohormone (PP) self-regulates pancreatic secretion activities and has effects on hepatic glucose levels through impaired hepatic insulin sensitivity [49]. Upregulations in PP secretion have been observed in pancreatic neuroendocrine tumors (PNETs) of the distal pancreas and of the gastrointestinal tract [50, 51]. HGFR/c-MET is involved in promoting tumor cell survival through ERK/MAPK, PAK, and mTOR signaling pathways [52]. Granzyme A (a serine protease) and FASL are deployed by cytotoxic T-lymphocytes (CTL) and natural killer (NK) cells to trigger tumor cell death [53, 54]. High levels of FASL in the aqueous humor (AH) were previously associated with poor survival in UM patients.

When comparing protein expression by PRAME status, there were 32 differentially expressed proteins at the p<0.01 level (FIG. 3B; Table 4). Among the upregulated proteins in PRAME positive patients were Desmoglein-3, Ectonucleotide pyrophosphatase/phosphodiesterase family member 2 (ENPP2, Autotaxin), Galectin-9 (LEG9), and Hepatocyte growth factor (HGF). Desmoglein-3 promotes cancer cell migration and invasion in squamous cell carcinoma by regulation activator protein 1 and PKC-dependent Ezrin activation [55]. Autotaxin is a tumor cell motility stimulating factor that promotes cell motility, proliferation, and angiogenesis through the generation lysophosphatidic acid (via lysophosphatidyl choline hydrolysis) [56-58]. Autotaxin is upregulated in several types of malignancies [59-61] (including lung, thyroid, and breast cancer) and its overexpression is associated with poor prognosis in UM patients [62]. Galectin-9 promotes differentiation of Th2 cells and M2 macrophages and is associated with poor prognosis for UM and cutaneous melanoma patients [63, 64]. Finally, HGF binds to the tyrosine kinase HGFR/c-MET receptor and promotes tumor cell survival [52]. Together, these data indicate that Siglec-6, c-MYC, OSM, SCFR/c-Kit, βc, HGFR/c-MET, Granzyme A, MEF2C, Arginase-1, FASL, PP, DNMT3A, Desmoglein-3, Autotaxin, Galectin-9, and HGF could serve as prognostic vitreous biomarkers for metastatic UM.

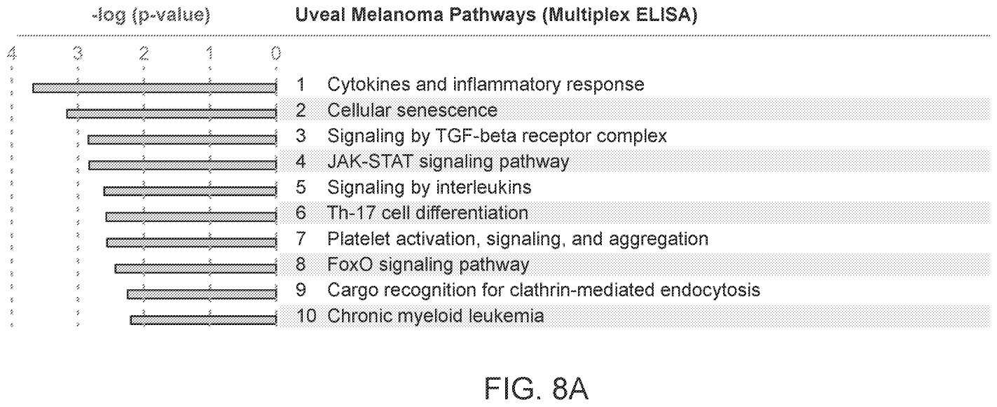

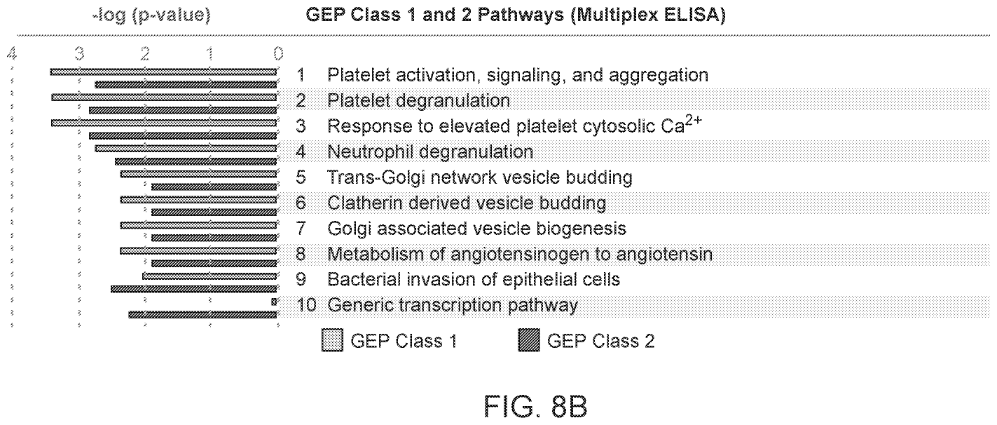

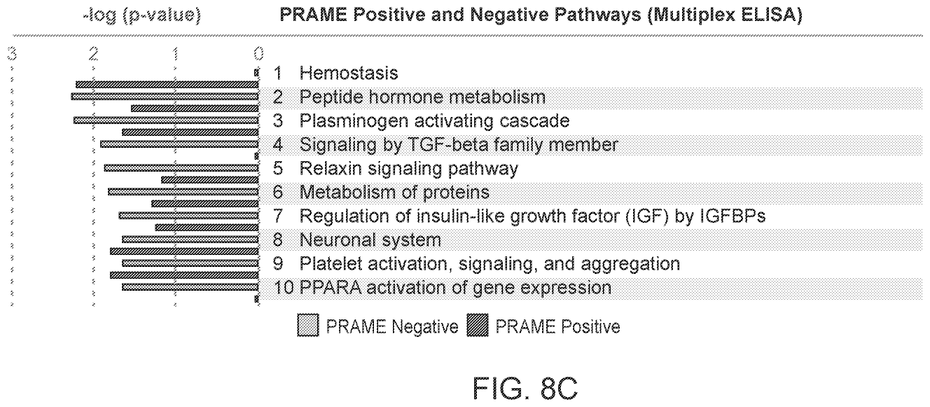

To classify differentially expressed proteins in UM vitreous, we performed pathway analysis, which identifies groups of functionally linked proteins (FIG. 8). Since the multiplex ELISA array only measured 1,000 proteins (and does not represent the entire proteome), we used an overrepresentation analysis (ORA)-based method with the 1,000 represented proteins as the reference protein list in WebGestalt [65, 66]. The top represented pathways in GEP Class 1 and 2 vitreous were platelet degranulation and activation, vesicle budding and biogenesis, and angiotensinogen metabolism (FIG. 8). (FIG. 8). The top represented pathways in PRAME positive vitreous were peptide hormone metabolism, plasminogen activating cascade, TGFβ signaling, and the Relaxin signaling pathway. The top represented pathways in PRAME negative vitreous were hemostasis, platelet activation, and neuronal system pathways (FIG. 8).

Liquid chromatography mass spectrometry identifies additional tumor markers not captured in targeted proteomics screens—Uveal melanoma and control vitreous samples were albumin-depleted and underwent trypsinization followed by multidimensional liquid chromatography before analysis by tandem mass spectrometry (Table 5). We identified 380±122 (mean±SD) individual proteins (6,600±2,469 spectra with 4,218±1,309 unique peptides) in UM vitreous and 253±88 individual proteins (2,844±2,405 spectra with 2,022±1,222 unique peptides) in control vitreous. The most abundant proteins identified in UM and control vitreous were Complement C3 (C3), Hemopexin (HPX), Ceruloplasmin (CP), and Vitamin D-binding protein (VTDB). Protein spectral counts were analyzed with 1-way ANOVA. A total of 69 proteins were differentially expressed among control and UM samples (64 upregulated proteins and 5 downregulated proteins; p<0.05; FIG. 7B; Table 6).

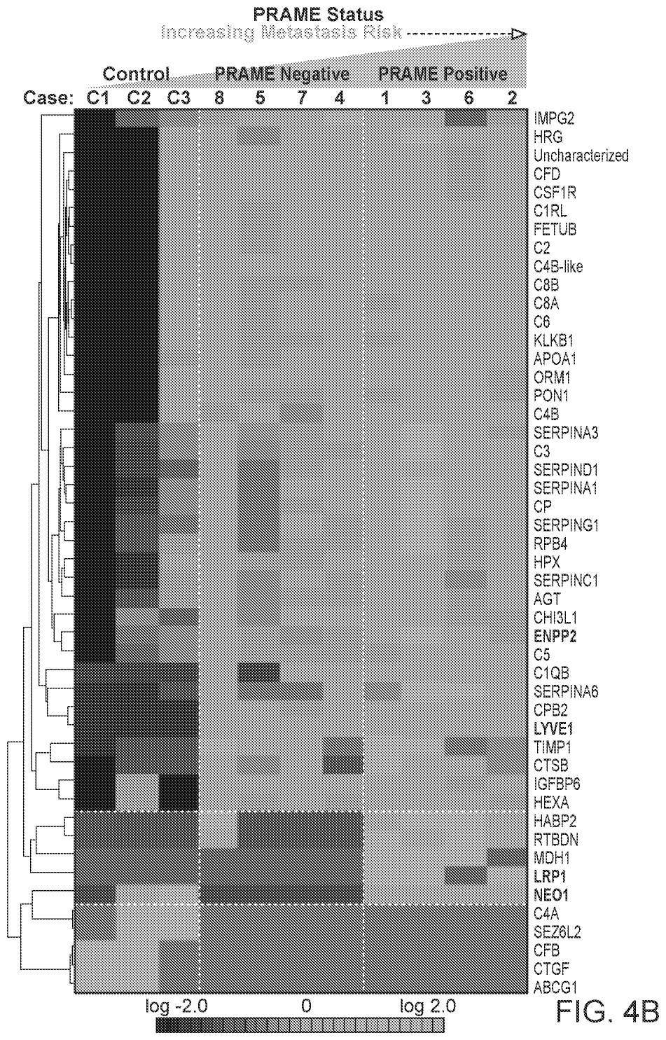

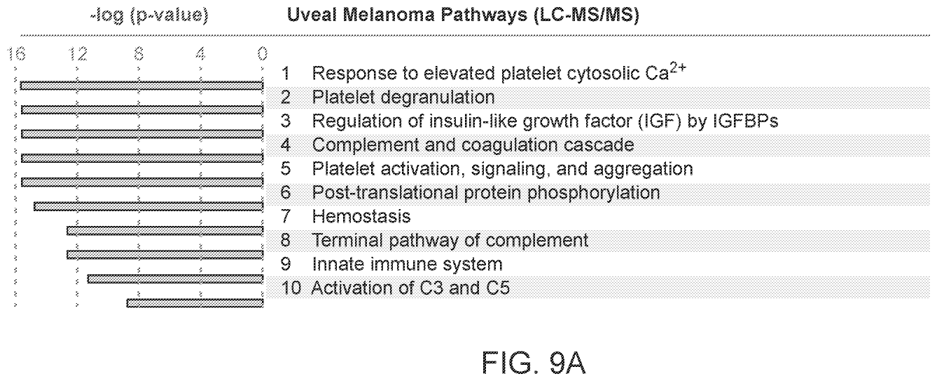

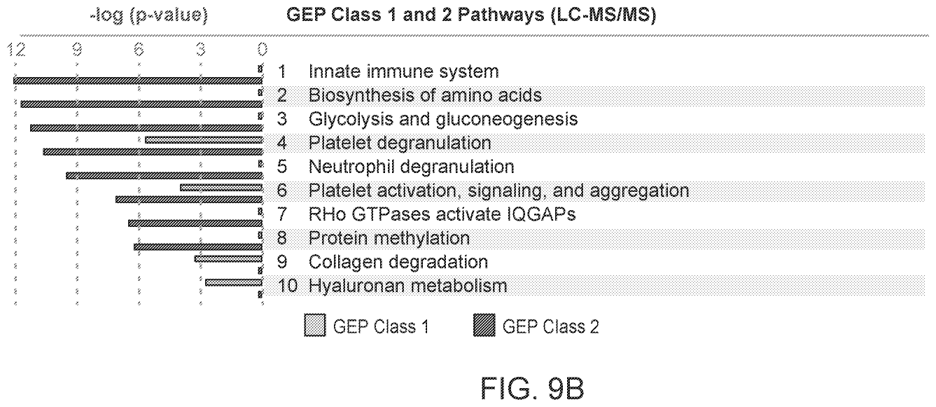

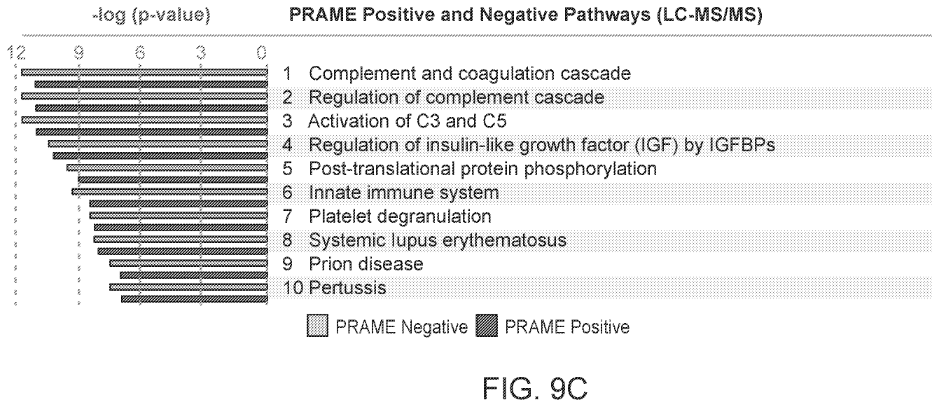

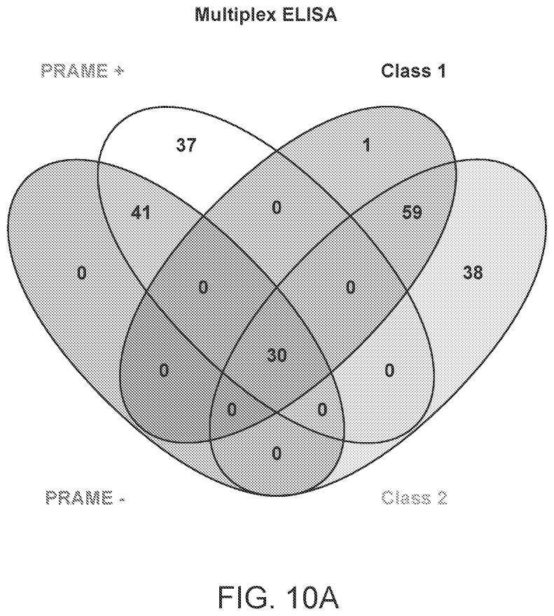

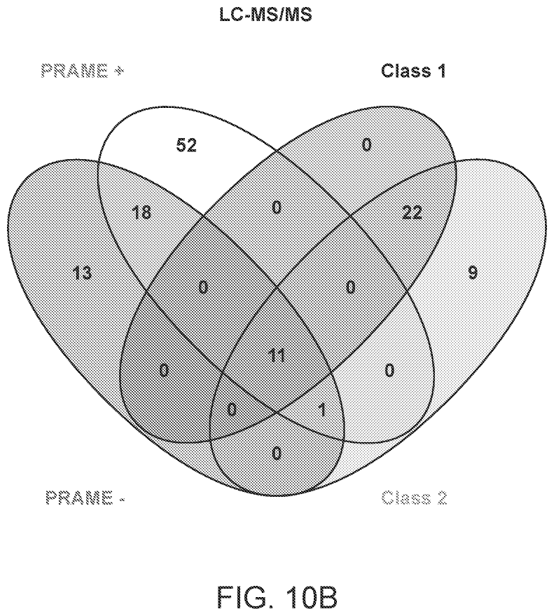

When comparing protein expression by GEP class, there were 53 differentially expressed proteins (43 upregulated proteins and 10 downregulated proteins) at the p<0.05 level (FIG. 4A). Among the upregulated proteins in GEP Class 2 vitreous were HGFR/c-MET and liver glycogen phosphorylase (PYGL; FIG. 4A). The top represented pathways in GEP Class 1 vitreous were platelet degranulation and activation, collagen degradation, and hyaluronan metabolism (FIG. 9). The top represented pathways in GEP class 2 vitreous were metabolic processes (glycolysis, gluconeogenesis, and amino acid biosynthesis), neutrophil degranulation, and innate immune system (FIG. 9). When comparing protein expression by PRAME status, there were 48 differentially expressed proteins (43 upregulated proteins and 5 downregulated proteins) at the p<0.05 level (FIG. 4B; Table 7). Among the upregulated proteins in PRAME positive vitreous were ENPP2, Neogenin (NEO1), and Pro-low-density lipoprotein receptor-related protein 1 (LRP1; FIG. 4B). The top represented pathways in PRAME positive and negative vitreous were complement cascade, coagulation cascade, and regulation of insulin-growth factor (IGF) by IGF-binding proteins (IGFBPs) (FIG. 9). The detection of ENPP2 and HGFR/c-MET on a separate proteomic platform further validated these proteins as vitreous biomarkers for UM. The high representation of metabolic pathways in GEP Class 2 and PRAME positive UM vitreous may be due to reprograming by the primary tumor to meet its increased metabolic demands [67].

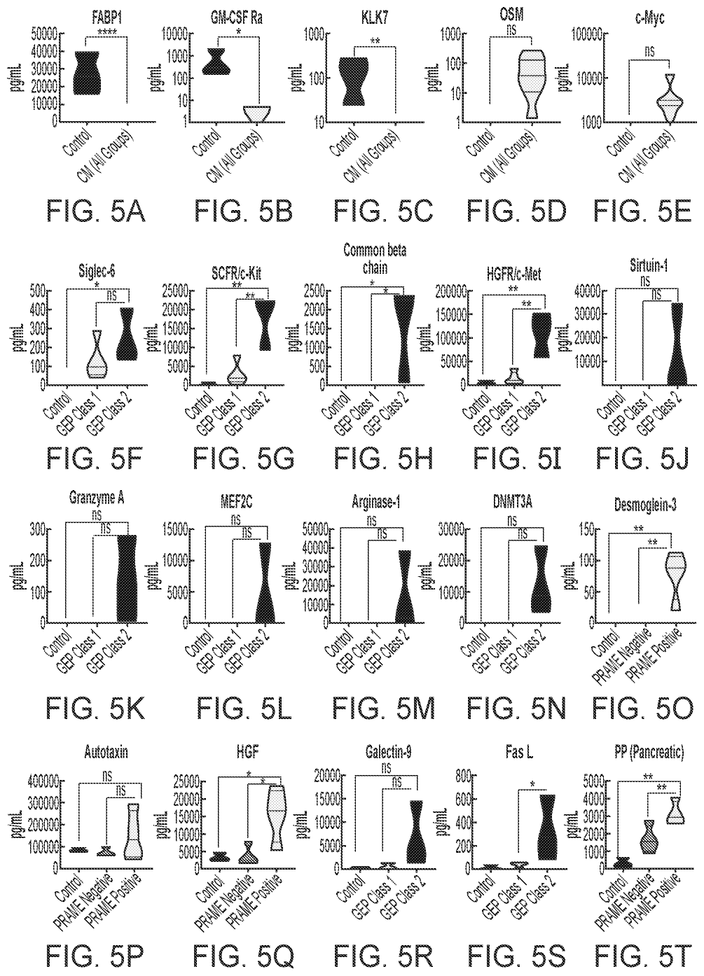

Biomarker Validation Study—The limited number of samples and high number of measurements can introduce bias and false positives in the training dataset. To validate the training dataset prospectively, 20 proteins were selected for further study based on their statistical significance, differential expression, dose response, and/or biological function (Table 2; FIG. 5)—FABP1, GM-CSF Ra, KLK7, Siglec-6, c-MYC, OSM, SCFR/c-Kit, βc, HGFR/c-MET, sirtuin-1, granzyme A, MEF2C, Arginase-1, FASL, PP, DNMT3A, Desmoglein-3, Autotaxin, Galectin-9, and HGF. We generated a custom multiplex ELISA array that measured the levels of these 20 proteins in vitreous and serum samples (FIG. 6).

### Discussion

Prognostic biomarkers in cancer medicine allow physicians to predict the natural course of disease in patients. UM, however, has no equivalent sensitive and specific molecular assay, so diagnosis relies heavily on routine body imaging, which can delay the diagnosis and still does not inform targeted treatment. Thus, there is a critical unmet need to develop rapid and precise diagnostic tools for earlier detection of metastatic UM and to identify targetable biomarkers which may delay or eliminate the risk of metastatic disease.

We worked under the assumption that our UM patients might benefit from already available therapeutics or natural compounds (FIG. 7). SCFR has been shown to be inhibited by imatinib (Gleevac) and may be a drug repositioning candidate for adjuvant UM therapy [32].

TABLE 1
Patient Demographics

| | | | | Surgical | | Tumor | |
|---|---|---|---|---|---|---|---|
| Case | Sex | Age | Eye | Indication | Diagnosis | Size | Comments |
| 1 | M | 67 | OS | Left eye | Uveal melanoma | 16 × 13.66 × | Large inferior uveal |
| | | | | enucleation with | OS | 5.8 mm (6.7 mm | melanoma tumor of with |
| | | | | orbital implant | Age-related | with overlying | associated exudative |
| | | | | | nuclear cataract | RD) | detachment |
| | | | | | OU | A scan height: | |
| | | | | | Oncocytoma of | 6.73 mm | |
| | | | | | kidney | | |
| 2 | F | 30 | OS | Left eye | Uveal melanoma | 6 × 7 × | Circumpapillary choroid |
| | | | | enucleation with | OS | 2.5 mm | melanoma tumor with |
| | | | | orbital implant | Monoallelic | | associated exudative |
| | | | | | mutation of BAP1 | | detachment. Visual |
| | | | | | gene | | symptoms, subretinal |
| | | | | | Left thyroid | | fluid, lipofuscin, |
| | | | | | nodule | | and 2 months of |
| | | | | | Family history of | | visual symptoms. |
| | | | | | malignant | | |
| | | | | | neoplasm of | | |
| | | | | | breast | | |
| | | | | | Family history of | | |
| | | | | | melanoma | | |
| 3 | M | 80 | OS | Left eye | Uveal melanoma | 12.7 × 11.22 × | Low-mid reflective macular |
| | | | | enucleation with | OS | 4.75 mm | domed choroid melanoma |
| | | | | orbital implant | Abnormal CT | A scan height: | lesion with associated |
| | | | | | lung screening | 4.12 mm | exudative detachment |
| | | | | | Abnormal PET | | |
| | | | | | scan of colon | | |
| | | | | | Autonomic | | |
| | | | | | dysfunction | | |
| | | | | | Hyponatremia | | |
| 4 | M | 47 | OD | Right eye plaque | Uveal melanoma | 11.48 × 11.75 × | Collar stud choroid |
| | | | | brachytherapy | OD | 7.5 mm | melanoma tumor |
| | | | | and vitrectomy | | A scan height: | encroaching edge of |
| | | | | | | 6.97 mm | macula with associated |
| | | | | | | | exudative detachment |
| 5 | M | 57 | OD | Right eye plaque | Uveal melanoma | 11 × 9 × | Superior nasal choroid |
| | | | | brachytherapy | OD | 3.99 mm | melanoma tumor, low |
| | | | | and vitrectomy | Type 2 diabetes | A scan height: | domed with internally |
| | | | | | Chronic kidney | 4.06 mm (with | reflective lesion and |
| | | | | | disease | mid-low internal | surface retinal pigment |
| | | | | | Elevated LFTs | reflectivity) | epithelium changes, only |
| | | | | | | | mild subretinal fluid |
| | | | | | | | exudation |
| 6 | M | 57 | OS | Left eye | Uveal melanoma | 21.8 × 16.65 × | Large temporal cilio-choroid |
| | | | | enucleation with | OS | 12.74 mm | melanoma, low mid internal |
| | | | | orbital implant | Uveal nevus OD | A scan height: | reflectivity with pulsation |
| | | | | | | 14.63 mm | and with associated |
| | | | | | | | exudative detachment |
| 7 | F | 35 | OS | Left eye plaque | Uveal melanoma | 15.6 × 15.9 × | Peripapillary choroid |
| | | | | brachytherapy | OS | 7.3 mm | melanoma tumor with |
| | | | | and vitrectomy | History of | A scan height: | associated exudative |
| | | | | | Henoch- | 7.6 mm | detachment |
| | | | | | Schönlein | | |
| | | | | | purpura | | |
| | | | | | Meibomian gland | | |
| | | | | | disease OS | | |
| 8 | M | 58 | OS | Left eye plaque | Uveal melanoma | 15.6 × 14.5 × | Collar stud configuration |
| | | | | brachytherapy | OS | 7.3 mm | choroid melanoma with |
| | | | | and vitrectomy | Malignant | A scan with low | associated exudative |
| | | | | | neoplasm of | internal | detachment |
| | | | | | prostate | reflectivity | |
| | | | | | Non-significant | | |
| | | | | | vitreous | | |
| | | | | | hemorrhage OS | | |
| | | | | | Senile nuclear | | |
| | | | | | sclerosis, bilateral | | |

TABLE 2
Vitreous biomarkers for validation: Significantly differentially
expressed proteins detected by multiplex ELISA and LC-MS/MS.

| Protein | UniProt | Full Protein Name | Patient Group |
|---|---|---|---|
| FABP1 | P07148 | Fatty acid-binding protein 1 | Control |
| GM-CSF Ra | P15509 | Granulocyte-macrophage colony-stimulating | Control |
| | | factor receptor | |
| KLK7 | P49862 | Kallikrein 7 | Control |
| SIGL6 | O43699 | Sialic acid-binding Ig-like lectin 6 | UM (All Groups) |
| MYC | P01106 | Myc proto-oncogene protein | UM (All Groups) |
| OSM | P13725 | Oncostatin-M | UM (All Groups) |
| SCFR/KIT | P10721 | Stem cell growth factor receptor Kit (Gleevac) | UM (All Groups) |
| CSF2RB | P32927 | Common beta chain | GEP Class 2 |
| c-MET/HGFR | P08581 | Hepatocyte growth factor receptor (c-MET) | GEP Class 2 |
| SIR1 | Q96EB6 | Sirtuin-1 | GEP Class 2 |
| GRAA | P12544 | Granzyme A | GEP Class 2 |
| MEF2C | Q06413 | Myocyte-specific enhancer factor 2C | GEP Class 2 |
| ARGI1 | P05089 | Arginase-1 | GEP Class 2 |
| FASLG | P48023 | Fas ligand | GEP Class 2 |
| PP/PAHO | P01298 | Pancreatic prohormone | GEP Class 2 |
| DNMT3A | Q9Y6K1 | DNA (cytosine-5)-methyltransferase 3A | GEP Class 2 |
| DSG3 | P32926 | Desmoglein-3 | PRAME Positive |
| ENPP2 | Q13822 | Autotaxin | PRAME Positive |
| LEG9 | O00182 | Galectin-9 | PRAME Positive |
| HGF | P14210 | Hepatocyte growth factor | PRAME Positive |

TABLE 3
Differentially expressed ELISA proteins based on GEP class

| | | p-value | Fold-Change | p-value | Fold-Change | p-value | Fold-Change |
|---|---|---|---|---|---|---|---|
| | p-value | (Class 1 vs. | (Class 1 vs. | (Class 2 vs. | (Class 1 vs. | (Class 2 vs. | (Class 2 vs. |
| Protein | (GEP) | Control) | Control) | Control) | Control) | Class 1) | Class 1) |
| FABP1 | 1.41E−11 | 3.15E−12 | −2.54E+07 | 6.52E−12 | −2.54E+07 | 1 | 1 |
| DNMT3A | 4.41E−09 | 1 | 1 | 2.04E−09 | 9.26E+06 | 9.89E−10 | 9.26E+06 |
| Siglec-6 | 8.36E−08 | 2.20E−08 | 107289 | 2.97E−08 | 221112 | 0.130566 | 2.0609 |
| Kallikrein 7 | 2.30E−07 | 5.17E−08 | −94797 | 1.06E−07 | −94797 | 1 | 1 |
| OMgp | 4.51E−07 | 4.82E−06 | −1107.98 | 0.0953236 | 3.3153 | 1.66E−06 | 3673.29 |
| Arginase 1 | 1.12E−06 | 1 | 1 | 5.14E−07 | 4.31E+06 | 2.52E−07 | 4.31E+06 |
| OSM | 1.13E−06 | 5.35E−07 | 15054.2 | 2.47E−07 | 153545 | 0.004188 | 10.1995 |
| MEF2C | 1.47E−06 | 1 | 1 | 6.73E−07 | 1.43E+06 | 3.30E−07 | 1.43E+06 |
| Common | 3.42E−06 | 1 | 1 | 1.56E−06 | 694048 | 7.70E−07 | 694048 |
| beta Chain | | | | | | | |
| Granzyme A | 4.74E−06 | 1 | 1 | 2.17E−06 | 68885.7 | 1.07E−06 | 68885.7 |
| Sirtuin 1 | 5.01E−06 | 1 | 1 | 2.29E−06 | 2.52E+06 | 1.13E−06 | 2.52E+06 |
| IL-8 | 1.21E−05 | 1 | 1 | 5.51E−06 | 6929.82 | 2.74E−06 | 6929.82 |
| CD27 | 4.64E−05 | 0.00356218 | 97.493 | 2.72E−05 | 88155.8 | 0.00036983 | 904.227 |
| MIS RII | 0.00014871 | 1 | 1 | 6.64E−05 | 186602 | 3.39E−05 | 186602 |
| MIP-1a | 0.000181 | 0.00391934 | 46.899 | 0.00019982 | 1261.94 | 0.00858141 | 26.9076 |
| HB-EGF | 0.00018311 | 0.120785 | −1.45494 | 0.00731298 | 2.41054 | 0.00059402 | 3.50718 |
| HO-1 | 0.00087724 | 0.367159 | 2.83411 | 0.0002973 | 2814.43 | 0.00037225 | 993.056 |
| IL-16 | 0.00099892 | 0.13305 | 11.6905 | 0.00117358 | 4641.8 | 0.00437172 | 397.057 |
| DAPP1 | 0.00111247 | 1 | 1 | 0.00048384 | 11893.8 | 0.0002569 | 11893.8 |
| GM-CSF Ra | 0.00206575 | 0.00033057 | −441737 | 0.00251104 | −26036.9 | 0.199065 | 16.9658 |
| MMP-13 | 0.0021909 | 0.0120799 | 8.67041 | 0.00034532 | 100.509 | 0.00660998 | 11.5922 |
| Cathepsins | 0.00224408 | 0.0460366 | 2.12959 | 0.00073699 | 7.19839 | 0.00589612 | 3.38018 |
| S100A13 | 0.00288434 | 0.897934 | −1.13859 | 0.0028471 | 128.51 | 0.00138508 | 146.32 |
| TGFa | 0.00368794 | 0.557453 | 2.29556 | 0.00493699 | 422.505 | 0.00616863 | 184.053 |
| GPR56 | 0.00371868 | 0.0490115 | 3.54843 | 0.00066421 | 30.7567 | 0.00483872 | 8.66767 |
| CES2 | 0.00384001 | 0.248021 | 18.7868 | 0.00131028 | 611555 | 0.00292267 | 32552.4 |
| SCF R | 0.0048514 | 0.0143891 | 9.89105 | 0.00080831 | 82.3164 | 0.0202408 | 8.32231 |
| PIGF | 0.00496676 | 0.252041 | 8.89605 | 0.00170705 | 14228.1 | 0.00396761 | 1599.38 |
| TRAIL R3 | 0.00558777 | 0.179859 | 2.26772 | 0.00175767 | 19.8055 | 0.00557742 | 8.73366 |
| TAFA1 | 0.0056455 | 0.00587704 | −7447.92 | 0.768706 | 2.17015 | 0.00383058 | 16163.1 |
| Galectin-9 | 0.00573014 | 0.366733 | 1.65751 | 0.00317524 | 12.8527 | 0.00581489 | 7.75421 |
| Cystatin B | 0.00617715 | 0.0452452 | 3.71738 | 0.00151394 | 20.3233 | 0.0162137 | 5.46709 |
| Angiogenin | 0.00653827 | 0.156838 | 1.25705 | 0.00153027 | 2.23333 | 0.0052991 | 1.77665 |
| EG-VEGF | 0.00673961 | 0.270493 | 12.5327 | 0.00231959 | 55330.8 | 0.00538301 | 4414.91 |
| Integrin | 0.0072314 | 0.00948558 | −78783.1 | 0.660244 | 5.06244 | 0.00488426 | 398835 |
| alpha 5 | | | | | | | |
| HGF R | 0.00753933 | 0.117975 | 2.73008 | 0.00138483 | 24.4099 | 0.00600158 | 8.94111 |
| PDGF Rb | 0.00812138 | 0.201648 | 37.6118 | 0.00249033 | 504296 | 0.00773788 | 13407.9 |
| Siglec-9 | 0.00850629 | 0.670143 | −2.6714 | 0.0629871 | 224.887 | 0.0231855 | 600.762 |
| ROBO3 | 0.00856288 | 0.0102727 | −7.56693 | 0.371439 | 1.85178 | 0.00267123 | 14.0123 |
| CHMP2B | 0.00862391 | 0.00797598 | 170223 | 0.00275923 | 1.37E+07 | 0.223205 | 80.4428 |
| GDF-15 | 0.00893597 | 0.320003 | 1.85641 | 0.00260876 | 18.6033 | 0.00527517 | 10.0211 |
| PDGF-AA | 0.00895789 | 0.0479921 | 3.1195 | 0.00193015 | 12.6881 | 0.0213928 | 4.06735 |
| Ephrin-B3 | 0.00959977 | 0.30057 | 25.4737 | 0.0219542 | 12367.2 | 0.0701194 | 485.49 |
| FUCA1 | 0.00962622 | 0.900785 | 1.04426 | 0.00715577 | 4.03284 | 0.00498028 | 3.8619 |
| Ferritin | 0.00967032 | 0.103758 | 6.00668 | 0.00205892 | 158.317 | 0.0112645 | 26.3569 |
| ANGPTL4 | 0.00994493 | 0.0153275 | 19.3751 | 0.00284933 | 102.16 | 0.116922 | 5.27275 |

TABLE 4
Differentially expressed ELISA proteins based on PRAME status

| | | p-value | Fold-Change | p-value | Fold-Change | p-value | Fold-Change |
|---|---|---|---|---|---|---|---|
| | p-value | (Positive vs. | (Positive vs. | (Negative vs. | (Negative vs. | (Positive vs. | (Positive vs. |
| Protein | (PRAME) | Control) | Control) | Control) | Control) | Negative) | Negative) |
| FABP1 | 1.51E−13 | 1.08E−13 | −2.54E+07 | 1.08E−13 | −2.54E+07 | 1 | 1 |
| Desmoglein-3 | 1.10E−09 | 1.44E−09 | 64708.6 | 1 | 1 | 7.83E−10 | 64708.6 |
| Kallikrein 7 | 9.91E−09 | 7.07E−09 | −94797 | 7.07E−09 | −94797 | 1 | 1 |
| Siglec-6 | 2.54E−08 | 1.84E−08 | 132115 | 1.78E−08 | 138910 | 0.921528 | −1.05143 |
| EphB6 | 1.91E−06 | 2.41E−06 | 392025 | 1 | 1 | 1.34E−06 | 392025 |
| OSM | 1.62E−05 | 7.49E−06 | 62026.4 | 1.92E−05 | 16662.3 | 0.227161 | 3.72255 |
| GM-CSF Ra | 0.0005392 | 0.0002212 | −441737 | 0.00071913 | −52842.5 | 0.29521 | −8.35951 |
| PP | 0.00094297 | 0.00031037 | 12.0526 | 0.0024403 | 6.01525 | 0.10617 | 2.00367 |
| Furin | 0.0012448 | 0.893675 | −1.02755 | 0.00118763 | −2.62709 | 0.00087648 | 2.55667 |
| Podoplanin | 0.00172609 | 0.0556448 | 261.629 | 0.0191317 | −1448.94 | 0.00052441 | 379086 |
| SOX15 | 0.00312145 | 0.0126317 | 1135.1 | 0.194913 | −22.4349 | 0.00107516 | 25465.7 |
| GFAP | 0.00339276 | 0.00162764 | 2.5416 | 0.288317 | 1.25578 | 0.00521598 | 2.02393 |
| ENPP-7 | 0.00372146 | 0.00191911 | 27149.8 | 0.356042 | 9.08256 | 0.00496639 | 2989.22 |
| ROBO4 | 0.00420071 | 0.135063 | 203.646 | 0.0239078 | −7283.9 | 0.00135808 | 1.48E+06 |
| Follistatin- | 0.00457415 | 0.00334798 | 7.08017 | 0.714967 | 1.19699 | 0.00373321 | 5.91498 |
| like 1 | | | | | | | |
| GATA-4 | 0.00617188 | 0.0019966 | 9.61E+06 | 0.0170277 | 45255.8 | 0.143762 | 212.385 |
| BAMBI | 0.00653629 | 0.00290163 | 4.68256 | 0.27579 | 1.53351 | 0.010882 | 3.05349 |
| SLITRK5 | 0.00655776 | 0.0035087 | 8.23222 | 0.438136 | 1.52357 | 0.0077185 | 5.40324 |
| SorCS3 | 0.00666277 | 0.00322331 | 3.27E+06 | 0.348879 | 36.5714 | 0.00930123 | 89424.4 |
| HAO-1 | 0.00679907 | 0.0127883 | 23347.2 | 0.00236015 | 980292 | 0.236209 | −41.9875 |
| GFR alpha-2 | 0.00694925 | 0.00724513 | 2.59236 | 0.909136 | −1.03189 | 0.00401526 | 2.67502 |
| Semaphorin 7A | 0.00758048 | 0.341038 | 1.30654 | 0.0195368 | −2.15784 | 0.00284354 | 2.8193 |
| IL-2 | 0.00764944 | 0.00274229 | −5367.21 | 0.0126477 | −626.183 | 0.282401 | −8.57132 |
| TROY | 0.00804147 | 0.467769 | −8.92378 | 0.00435513 | −79606.3 | 0.0090638 | 8920.69 |
| TPP1 | 0.00831942 | 0.315778 | 1.22212 | 0.0229781 | −1.69214 | 0.00305067 | 2.068 |
| PDX-1 | 0.00837276 | 0.00304391 | 1.93E+06 | 0.0131292 | 57747 | 0.304741 | 33.3681 |
| CHMP2B | 0.00845654 | 0.00307648 | 1.00E+07 | 0.013223 | 202365 | 0.3061 | 49.5443 |
| CrkL | 0.00913102 | 0.00334335 | 88382.8 | 0.0139186 | 5796.01 | 0.318142 | 15.2489 |
| Glypican 1 | 0.00983008 | 0.0124924 | 6.10165 | 0.729242 | −1.22411 | 0.00487412 | 7.46911 |

TABLE 5
Mass spectrometry overview

| | | | | | Spectrum-level |
|---|---|---|---|---|---|
| Patient | Condition | Spectra | Peptide | Proteins | FDR (%) |
| 1 | UM | 7,603 | 4,573 | 349 | 1.1 |
| 2 | UM | 4,863 | 2,997 | 255 | 0.5 |
| 3 | UM | 6,858 | 4,367 | 384 | 1.7 |
| 4 | UM | 5,877 | 3,656 | 300 | 0.9 |
| 5 | UM | 11,626 | 6,755 | 620 | 1.2 |
| 6 | UM | 3,599 | 2,389 | 275 | 0.7 |
| 7 | UM | 8,586 | 4,717 | 493 | 0.6 |
| 8 | UM | 6,500 | 4,292 | 438 | 0.7 |
| 9 | ERM | 2,557 | 1,915 | 287 | 0.7 |
| 10 | ERM | 5,697 | 3,293 | 336 | 0.7 |
| 11 | ERM | 1,061 | 857 | 173 | 0.5 |

TABLE 6
Differentially expressed LC-MS/MS proteins based on GEP class

| | | p-value | Fold-Change | p-value | Fold-Change | p-value | Fold-Change |
|---|---|---|---|---|---|---|---|
| | p-value | (Class 1 vs. | (Class 1 vs. | (Class 2 vs. | (Class 1 vs. | (Class 2 vs. | (Class 2 vs. |
| Protein | (GEP) | Control) | Control) | Control) | Control) | Class 1) | Class 1) |
| ARPC3 | 0 | 1 | 1 | 0 | 1000 | 0 | −1000 |
| PLDX1 | 0 | 0 | 31.6228 | 1 | 1 | 0 | 31.6228 |
| ALDOA | 5.51E−12 | 1 | 1 | 2.56E−12 | 19349.9 | 1.24E−12 | −19349.9 |
| PRDX1 | 3.41E−11 | 1 | 1 | 1.58E−11 | 18469.1 | 7.64E−12 | −18469.1 |
| TGON2 | 3.74E−11 | 3.90E−10 | 49.4923 | 1 | 1 | 3.90E−10 | 49.4923 |
| LAMP2 | 3.74E−11 | 3.90E−10 | 49.4923 | 1 | 1 | 3.90E−10 | 49.4923 |
| PRDX2 | 1.69E−10 | 1 | 1 | 7.85E−11 | 11538 | 3.80E−11 | −11538 |
| CALM1 | 7.04E−10 | 1 | 1 | 3.26E−10 | 7910.46 | 1.58E−10 | −7910.46 |
| TKT | 1.21E−09 | 1 | 1 | 5.60E−10 | 16411.2 | 2.71E−10 | −16411.2 |
| HBA | 1.22E−09 | 1 | 1 | 5.66E−10 | 13722.4 | 2.74E−10 | −13722.4 |
| GPNMB | 1.49E−09 | 2.77E−07 | 31.6228 | 8.74E−10 | 6542.13 | 1.37E−08 | −206.88 |
| NPM | 2.37E−09 | 1 | 1 | 1.10E−09 | 1587.4 | 5.31E−10 | −1587.4 |
| PROF1 | 2.37E−09 | 1 | 1 | 1.10E−09 | 1587.4 | 5.31E−10 | −1587.4 |
| CHL1 | 2.64E−09 | 2.72E−08 | 37.606 | 1 | 1 | 2.72E−08 | 37.606 |
| CAPG | 2.95E−09 | 1 | 1 | 1.37E−09 | 1259.92 | 6.63E−10 | −1259.92 |
| MDHM | 3.36E−09 | 1 | 1 | 1.55E−09 | 3107.23 | 7.54E−10 | −3107.23 |
| FABP5 | 4.08E−09 | 1 | 1 | 1.89E−09 | 10208.9 | 9.17E−10 | −10208.9 |
| CAP1 | 4.23E−09 | 1 | 1 | 1.96E−09 | 3979.06 | 9.49E−10 | −3979.06 |
| PPIA | 5.13E−09 | 1 | 1 | 2.38E−09 | 9435.39 | 1.15E−09 | −9435.39 |
| HBB | 8.09E−09 | 1 | 1 | 3.74E−09 | 18686.5 | 1.82E−09 | −18686.5 |
| GSTP1 | 1.05E−08 | 1 | 1 | 4.86E−09 | 12480.5 | 2.36E−09 | −12480.5 |
| FSCN1 | 1.20E−08 | 1 | 1 | 5.57E−09 | 3301.93 | 2.70E−09 | −3301.93 |
| PSA | 2.05E−08 | 1 | 1 | 9.48E−09 | 1817.12 | 4.60E−09 | −1817.12 |
| ZRAB3 | 2.05E−08 | 1 | 1 | 9.48E−09 | 1817.12 | 4.60E−09 | −1817.12 |
| THIO | 2.41E−08 | 1 | 1 | 1.11E−08 | 4326.75 | 5.41E−09 | −4326.75 |
| 1433Z | 3.36E−08 | 1 | 1 | 1.55E−08 | 9205.16 | 7.55E−09 | −9205.16 |
| HSPB1 | 4.48E−08 | 1 | 1 | 2.07E−08 | 6868.29 | 1.01E−08 | −6868.29 |
| IDHC | 5.55E−08 | 1 | 1 | 2.57E−08 | 5241.48 | 1.25E−08 | −5241.48 |
| RPN1 | 6.41E−08 | 1 | 1 | 2.96E−08 | 1442.25 | 1.44E−08 | −1442.25 |
| VATB2 | 6.41E−08 | 1 | 1 | 2.96E−08 | 1442.25 | 1.44E−08 | −1442.25 |
| PYGL | 7.64E−08 | 1 | 1 | 3.53E−08 | 10208.9 | 1.72E−08 | −10208.9 |
| ACTN4 | 1.07E−07 | 1 | 1 | 4.94E−08 | 5039.68 | 2.40E−08 | −5039.68 |
| H2B2E | 1.13E−07 | 1 | 1 | 5.19E−08 | 2289.43 | 2.53E−08 | −2289.43 |
| TBA1B | 1.17E−07 | 1 | 1 | 5.39E−08 | 5517.85 | 2.62E−08 | −5517.85 |
| AATC | 1.57E−07 | 1 | 1 | 7.26E−08 | 3174.8 | 3.54E−08 | −3174.8 |
| TERA | 1.77E−07 | 1 | 1 | 8.15E−08 | 5451.36 | 3.97E−08 | −5451.36 |
| EF2 | 1.80E−07 | 1 | 1 | 8.29E−08 | 3914.87 | 4.04E−08 | −3914.87 |
| HSP7C | 2.42E−07 | 1 | 1 | 1.12E−07 | 19892.8 | 5.44E−08 | −19892.8 |
| EF1G | 2.93E−07 | 1 | 1 | 1.35E−07 | 1587.4 | 6.60E−08 | −1587.4 |
| ANXA5 | 3.78E−07 | 1 | 1 | 1.74E−07 | 6231.68 | 8.50E−08 | −6231.68 |
| GDIA | 4.05E−07 | 1 | 1 | 1.87E−07 | 5808.79 | 9.11E−08 | −5808.79 |
| HS71A | 4.54E−07 | 1 | 1 | 2.09E−07 | 6382.5 | 1.02E−07 | −6382.5 |
| PEBP1 | 4.85E−07 | 1 | 1 | 2.23E−07 | 5646.22 | 1.09E−07 | −5646.22 |
| GLNA | 5.83E−07 | 1 | 1 | 2.68E−07 | 6782.42 | 1.31E−07 | −6782.42 |
| SEZ6 | 8.36E−07 | 0.353026 | 1.56724 | 4.70E−07 | −6782.42 | 1.64E−07 | 10629.7 |
| LYVE1 | 1.01E−06 | 2.10E−07 | 2397.13 | 5.04E−07 | 2000 | 0.659021 | 1.19857 |
| TBB4B | 1.53E−06 | 1 | 1 | 7.02E−07 | 13313.9 | 3.44E−07 | −13313.9 |
| CAH2 | 2.08E−06 | 1 | 1 | 9.54E−07 | 14765.2 | 4.69E−07 | −14765.2 |
| MOES | 3.80E−06 | 1 | 1 | 1.74E−06 | 4626.07 | 8.57E−07 | −4626.07 |
| TALDO | 4.42E−06 | 1 | 1 | 2.02E−06 | 4702.67 | 9.96E−07 | −4702.67 |
| CBPB2 | 6.69E−06 | 1.41E−06 | 8210.75 | 3.27E−06 | 6854.12 | 0.772593 | 1.19793 |
| ATPB | 7.46E−06 | 1 | 1 | 3.40E−06 | 5646.22 | 1.68E−06 | −5646.22 |
| PARK7 | 1.60E−05 | 1 | 1 | 7.26E−06 | 7211.25 | 3.61E−06 | −7211.25 |
| TBA1A | 4.89E−05 | 1 | 1 | 2.20E−05 | 12082.8 | 1.11E−05 | −12082.8 |
| VIME | 0.00232051 | 0.330681 | 5.62341 | 0.00077287 | 31581.8 | 0.00121891 | −5616.13 |
| ACTG | 0.00344909 | 0.211469 | −10 | 0.00429891 | 2225.06 | 0.00055407 | −22250.6 |
| LDHB | 0.00348419 | 0.283957 | 5.62341 | 0.00116942 | 5943.92 | 0.00226473 | −1057 |
| ALDOC | 0.00388738 | 0.199273 | −10 | 0.00512482 | 1375.07 | 0.00062038 | −13750.7 |
| BIP | 0.00601326 | 0.250003 | 8.40896 | 0.00208774 | 7651.72 | 0.00510191 | −909.949 |
| CATB | 0.00776261 | 0.00674366 | 1.88402 | 0.00431148 | 2.15443 | 0.447876 | −1.14353 |
| MET | 0.00803071 | 0.489387 | 3.54954 | 0.00229404 | 8041.45 | 0.00297627 | −2265.49 |
| NTRI | 0.0108175 | 0.280768 | −7.08506 | 0.00362564 | −2884.5 | 0.00889423 | 407.124 |
| TIMP1 | 0.0117672 | 0.00378267 | 2.87779 | 0.0109492 | 2.57687 | 0.67023 | 1.11678 |
| SUN3 | 0.0123241 | 0.0419823 | 49.4923 | 0.0333101 | 100 | 0.667884 | −2.02052 |
| LCAT | 0.0129995 | 0.249032 | 8.40896 | 0.00477418 | 2080.08 | 0.0139886 | −247.365 |
| HEP2 | 0.0131447 | 0.0037774 | 7.63722 | 0.00484062 | 8.58671 | 0.813366 | −1.12432 |
| C163A | 0.0135788 | 0.411674 | 5.21491 | 0.030701 | 288.45 | 0.0716195 | −55.3125 |
| C1QB | 0.0136473 | 0.00627309 | 1258.24 | 0.00411784 | 5428.84 | 0.455942 | −4.31461 |
| MIF | 0.0140179 | 0.187594 | −10 | 0.0230948 | 158.74 | 0.00227718 | −1587.4 |
| PVR | 0.014924 | 0.130951 | 14.9831 | 0.0342721 | −100 | 0.00242619 | 1498.31 |
| GPR37 | 0.0168431 | 0.323858 | 5.3183 | 0.229024 | −10 | 0.0396229 | 53.183 |
| CO9A1 | 0.0180197 | 0.0255633 | 181.712 | 0.232633 | −14.4225 | 0.00367679 | 2620.74 |
| CRDL1 | 0.0184874 | 0.592428 | −2.89273 | 0.00596632 | −3556.89 | 0.00711126 | 1229.6 |
| LMAN2 | 0.0192862 | 0.00805335 | 667.16 | 1 | 1 | 0.00805335 | 667.16 |
| GOLM1 | 0.0206698 | 0.493621 | −3.46545 | 0.0352346 | −144.225 | 0.0669706 | 41.6179 |
| NOE2 | 0.0212674 | 0.00424029 | 2240.49 | 0.235642 | 14.4225 | 0.0297208 | 155.347 |
| GLU2B | 0.0232166 | 0.0765834 | 53.183 | 0.0335043 | 271.442 | 0.422718 | −5.10392 |
| A1AT | 0.02445 | 0.00844675 | 7.95134 | 0.00674316 | 11.1199 | 0.575954 | −1.39849 |
| FSTL5 | 0.0261041 | 0.0547351 | 37.606 | 0.229024 | 10 | 0.428097 | 3.7606 |
| CO9A3 | 0.0265538 | 0.0554281 | 53.183 | 0.228751 | 12.5992 | 0.433091 | 4.22113 |
| TNR16 | 0.0274876 | 0.0899738 | 41.6179 | 0.0339859 | 251.984 | 0.373881 | −6.05471 |
| LBP | 0.027528 | 0.0899341 | 37.606 | 0.0341196 | 215.443 | 0.375446 | −5.72896 |
| LSAMP | 0.0284361 | 0.946164 | 1.12057 | 0.0120638 | −430.887 | 0.00672402 | 482.841 |
| CBG | 0.0300477 | 0.0128362 | 3.5905 | 0.007032 | 5.00296 | 0.417984 | −1.39339 |
| IC1 | 0.0300888 | 0.012865 | 2.89156 | 0.0107277 | 3.40514 | 0.625514 | −1.17762 |
| NPVF | 0.030459 | 0.0611216 | 51.4369 | 0.237533 | 12.5992 | 0.452438 | 4.08255 |
| TPIS | 0.0315213 | 0.230543 | −10.5444 | 0.255212 | 11.7767 | 0.0311812 | −124.179 |
| ENOA | 0.0345943 | 0.214916 | 26.5811 | 0.0132613 | 6541.63 | 0.0559057 | −246.101 |
| HEXA | 0.0372743 | 0.0152509 | 156.508 | 0.00916333 | 524.148 | 0.470215 | −3.34901 |
| LYAG | 0.0398269 | 0.0744735 | 37.606 | 0.228751 | 12.5992 | 0.547799 | 2.98479 |
| PGK1 | 0.0402188 | 0.197702 | −28.1727 | 0.0719907 | 247.277 | 0.00696247 | −6966.46 |
| PGAM1 | 0.0404845 | 0.799068 | −1.77828 | 0.0307424 | 675.331 | 0.0139176 | −1200.93 |
| T132A | 0.0436807 | 0.0272572 | 219.921 | 1 | 1 | 0.0272572 | 219.921 |
| IBP6 | 0.0463056 | 0.00969056 | 658.905 | 0.0257147 | 319.67 | 0.706313 | 2.0612 |
| CERU | 0.0472586 | 0.0145069 | 3.22199 | 0.0155815 | 3.58696 | 0.77583 | −1.11327 |
| APLP2 | 0.0475523 | 0.484415 | 1.36857 | 0.0371711 | −3.35512 | 0.0089047 | 4.59172 |
| PMEL | 0.0479568 | 0.203499 | 31.2399 | 0.00977178 | 14374.3 | 0.0410664 | −460.125 |
| GDF8 | 0.0493665 | 0.0297319 | 142.816 | 1 | 1 | 0.0297319 | 142.816 |

TABLE 7
Differentially expressed LC-MS/MS proteins based on PRAME status

| | | p-value | Fold-Change | p-value | Fold-Change | p-value | Fold-Change |
|---|---|---|---|---|---|---|---|
| | p-value | (Positive vs. | (Positive vs. | (Negative vs. | (Negative vs. | (Positive vs. | (Positive vs. |
| Protein | (PRAME) | Control) | Control) | Control) | Control) | Negative) | Negative) |
| LYVE1 | 1.11E−07 | 7.69E−08 | 2213.36 | 8.06E−08 | 2114.74 | 0.909061 | 1.04664 |
| CBPB2 | 2.48E−07 | 1.33E−07 | 10586 | 2.46E−07 | 5243.61 | 0.196787 | 2.01883 |
| HEP2 | 0.00396439 | 0.00153618 | 9.55474 | 0.0054026 | 6.13069 | 0.347227 | 1.55851 |
| A1AT | 0.00504535 | 0.0018285 | 11.7773 | 0.00822331 | 6.57127 | 0.276609 | 1.79223 |
| RTBDN | 0.0056447 | 0.00268754 | 2213.36 | 0.321848 | 6.6874 | 0.00829038 | 330.975 |
| NEO1 | 0.00638419 | 0.306183 | 8.37579 | 0.0180747 | −317.48 | 0.00234689 | 2659.15 |
| CBG | 0.00649955 | 0.00229057 | 4.96316 | 0.0115348 | 3.27771 | 0.253474 | 1.51421 |
| C1QB | 0.00677611 | 0.0023246 | 5885.66 | 0.01341 | 518.004 | 0.221163 | 11.3622 |
| HABP2 | 0.0102481 | 0.0047134 | 3389.56 | 0.339877 | 8.40896 | 0.0149254 | 403.089 |
| CERU | 0.0119845 | 0.00439766 | 3.87791 | 0.0178832 | 2.78937 | 0.333051 | 1.39024 |
| MDHC | 0.0143377 | 0.012785 | 640.217 | 1 | 1 | 0.00873807 | 640.217 |
| IBP6 | 0.0146973 | 0.010623 | 398.58 | 0.00769654 | 591.377 | 0.81943 | −1.48371 |
| HEXA | 0.015618 | 0.00745138 | 313.017 | 0.0128193 | 173.205 | 0.702823 | 1.8072 |
| LRP1 | 0.0166475 | 0.0146603 | 559.508 | 1 | 1 | 0.0101005 | 559.508 |
| APOA1 | 0.0172297 | 0.00807292 | 2165.9 | 0.0144526 | 918.762 | 0.684024 | 2.35741 |
| CO3 | 0.0184866 | 0.0075124 | 4.7142 | 0.0202844 | 3.52965 | 0.494618 | 1.3356 |
| AACT | 0.0195689 | 0.0079614 | 4.59567 | 0.0213381 | 3.45629 | 0.498889 | 1.32965 |
| E7ETN3 | 0.0222985 | 0.0141321 | 220.899 | 0.0127549 | 248.442 | 0.943239 | −1.12468 |
| CO2 | 0.0223198 | 0.0123563 | 515.177 | 0.0146719 | 412.869 | 0.905096 | 1.2478 |
| IC1 | 0.0238053 | 0.00955859 | 3.33707 | 0.0264724 | 2.62782 | 0.489044 | 1.2699 |
| B4E1Z4 | 0.0263437 | 0.0145933 | 2934.81 | 0.0171244 | 2238.32 | 0.912251 | 1.31117 |
| CO8B | 0.0270059 | 0.0122176 | 906.862 | 0.023299 | 370.038 | 0.659157 | 2.45073 |
| ABCG1 | 0.0275496 | 0.0164661 | −100 | 0.0164661 | −100 | 1 | 1 |
| CATB | 0.0277929 | 0.00950369 | 2.18742 | 0.0589668 | 1.66208 | 0.234831 | 1.31607 |
| CCN2 | 0.0279982 | 0.0167263 | −125.992 | 0.0167263 | −125.992 | 1 | 1 |
| CFAB | 0.0286819 | 0.0171226 | −711.379 | 0.0171226 | −711.379 | 1 | 1 |
| TIMP1 | 0.0295482 | 0.0149975 | 2.73512 | 0.0212518 | 2.53887 | 0.811424 | 1.0773 |
| SEZ6L2 | 0.0297155 | 0.0177213 | −170.998 | 0.0177213 | −170.998 | 1 | 1 |
| CO4A | 0.0306002 | 0.0182333 | −191.293 | 0.0182333 | −191.293 | 1 | 1 |
| A1AG1 | 0.0313898 | 0.0166789 | 404.08 | 0.0212028 | 295.717 | 0.869667 | 1.36644 |
| FETUB | 0.0321783 | 0.0150577 | 556.988 | 0.0259379 | 268.984 | 0.711466 | 2.07071 |
| KLKB1 | 0.0326125 | 0.0164548 | 737.448 | 0.0234959 | 443.924 | 0.808087 | 1.6612 |
| CSF1R | 0.0327568 | 0.0207155 | 220.463 | 0.0183747 | 255.361 | 0.934713 | −1.15829 |
| HEMO | 0.0342256 | 0.0152793 | 5.17441 | 0.0297823 | 4.10144 | 0.651409 | 1.26161 |
| CO6 | 0.0353266 | 0.0178015 | 783.414 | 0.0253672 | 467.318 | 0.809682 | 1.6764 |
| ENPP2 | 0.0361527 | 0.0130499 | 3.21791 | 0.0565702 | 2.26853 | 0.334654 | 1.4185 |
| CFAD | 0.0383573 | 0.0208925 | 265.286 | 0.0248454 | 213.138 | 0.9063 | 1.24467 |
| CO8A | 0.0401553 | 0.0210222 | 657.49 | 0.0272129 | 450.079 | 0.861099 | 1.46083 |
| HRG | 0.0414177 | 0.0158959 | 6.63612 | 0.0511423 | 4.151 | 0.438184 | 1.59868 |
| CO4B | 0.0424746 | 0.0166843 | 3585.98 | 0.0488303 | 545.178 | 0.475019 | 6.57764 |
| CO5 | 0.0426529 | 0.0205665 | 173.138 | 0.032247 | 102.834 | 0.761374 | 1.68367 |
| C1RL | 0.0431172 | 0.0222669 | 265.286 | 0.0296067 | 184.378 | 0.847202 | 1.43882 |
| CH3L1 | 0.0431626 | 0.0222801 | 5.37618 | 0.0296538 | 4.81594 | 0.846678 | 1.11633 |
| ANT3 | 0.0440501 | 0.0205317 | 3.49723 | 0.0350557 | 3.00913 | 0.718543 | 1.1622 |
| IMPG2 | 0.04674 | 0.05247 | 2.26259 | 0.0184995 | 2.87997 | 0.488452 | −1.27286 |
| PON1 | 0.0467414 | 0.0244074 | 357.555 | 0.0314962 | 252.046 | 0.863329 | 1.41861 |
| RET4 | 0.0467574 | 0.018082 | 3.94858 | 0.0562973 | 2.81185 | 0.451754 | 1.40426 |
| ANGT | 0.0490342 | 0.0192104 | 3.98443 | 0.0566805 | 2.86533 | 0.473053 | 1.39057 |

### Materials and Methods

Study approval—The study was approved by the Stanford University Institutional Review Board and adhered to the tenets set forth in the Declaration of Helsinki.

Clinical examination—Clinical examination and testing were performed, and the following data were collected: patient age (at the time of surgery), sex, tumor diameter (measured by B-scan ultrasonography), tumor thickness (measured by A- or B-scan ultrasonography). Patients were assessed for the presence or absence of exudative retinal detachment (RD), lipofuscin, drusen, retinal pigment epithelial fibrosis and epithelial atrophy, and low internal reflectivity (determined using A- or B-scan). Tumors were staged according to the American Joint Committee on Cancer (AJCC) classification [22, 23]. The GEP profile of the tumor samples was determined as previously described [68, 69]. Briefly, a fine-needle aspiration (FNA) of the tumor was performed and the aspirated contents were added to an empty tube. The needle hub was flushed with 200 μL of extraction buffer which was then expelled into the same tube. Tumor samples underwent RNA extraction followed by reverse transcription to generate cDNA and by analysis by real-time quantitative PCR. Data were analyzed by the certified laboratory of Castle Biosciences (Friendswood, Tex.), under the trade name DecisionDx-UM [68, 69]. The PRAME status of the tumor samples was determined by measuring PRAME mRNA expression on an Illumina HT-12v4 chip using probe ILMN_1700031 as described previously [70].

Vitreous sample collection—Pars plana vitrectomy was performed using a single-step transconjunctival 23-gauge trocar cannular system (Alcon Laboratories Inc, Fort Worth, Tex.), and an undiluted 0.5-cc sample of the vitreous was manually aspirated into a 3-cc syringe. Vitreous samples were immediately centrifuged in the operating room at 15,000×g for 5 minutes at room temperature to remove impurities and then finally stored at −80° C., as previously described [71].

Multiplex ELISA array—Vitreous cytokine signaling proteins were measured using the Human Kiloplex Array Q1 (RayBio, Norcross, Ga.) per the manufacturers protocol. Vitreous samples were diluted in phosphate-buffered saline (PBS; pH 7.4) to a final volume of 1.5 mL. This array concurrently detected and processed 1,000 human proteins. First, the array chips were incubated with sample diluents for 30 minutes at room temperature to act as a block. Diluted vitreous (four technical replicates per sample) was added to the wells of the array and incubated overnight at 4° C. A standard protein dilution was added to the wells of the array to determine protein concentrations. For signal detection, 80 μL of Cy3-streptavidin was added to each well, rinsed and visualized by laser scanner. The RayBio® Analysis Tool (RayBio®, Norcross, Ga.) was used for protein classification. Final protein concentrations (in pg/mL) were corrected for sample dilution.

Protein extraction, digestion, and peptide desalting—A shotgun proteomics screen was performed to identify biomarkers not sampled in our multiplex ELISA. Vitreous humor samples were albumin-depleted using affinity chromatography. Briefly, 100 μL of undiluted vitreous was loaded onto midi columns containing Top14 Abundant Protein Depletion resin (Thermo Scientific) and incubated at room temperature for 10 minutes with gentle mixing. Columns were then placed in 15 mL conical tubes and centrifuged at 1,000×g for 2 minutes and the filtrate discarded. Unbound proteins were eluted in 10 mM PBS (pH 7.4) and 0.02% sodium azide and protein concentration was determined by Bradford assay. A total of 5 μg of protein per sample was diluted in 50 mM ammonium bicarbonate to a final volume of 1 mL and reduced by addition of 10 mM DTT followed by incubation at 55° C. for 30 minutes. Alkylation was performed by adding 1 M acrylamide for a final concentration of 30 mM and incubating at room temperature for 30 minutes. Trypsin (0.5 μg) was then added to each tube and samples were digested overnight at 37° C. The reaction was quenched by adding 50% formic acid to a final concentration of 2%. Digested peptides were desalted using C18 stop-and-go extraction (STAGE) tips. Briefly, for each sample, a C18 STAGE tip was equilibrated with 0.1% trifluoroacetic acid (TFA) followed by 50% acetonitrile (ACN). Samples were loaded onto the tips and desalted with 50% ACN. Peptides were eluted with 0.1% formic acid in 50% ACN and lyophilized in a SpeedVac to dryness.

Liquid chromatography-tandem mass spectrometry—Peptide pools were reconstituted in 15 μl of 2% ACN and 0.1% formic acid solution. A total of 3 μL of each sample was injected into an in-house packed C18 reverse phase analytical column (15 cm in length). Ultra-performance liquid chromatography (UPLC) was performed on a Waters M-Class at a flow rate of 0.45 μL/min using a linear gradient from 4-40% Mobile phase B (0.2% formic acid, 99.8% ACN. Mobile phase A consisted of 0.2% formic acid. Mass spectrometry was performed on an Orbitrap fusion set to acquire data in a dependent fashion using the top-speed functionality. Fragmentation was performed on the most intense multiply charged precursor ions using collision induced dissociation (CID). MS data were analyzed using Preview and Byonic software (ProteinMetrics) to identify peptides and infer proteins using the Uniprot Homo sapiens annotated databases files including isoforms, concatenated with common contaminant proteins. Analysis was performed at 12 ppm mass tolerances for precursor ions, with 0.4 Da windows for fragment ions; only peptides with fully tryptic cleavages were tolerated, with up to two missed cleavage sites. Data were validated using the standard reverse-decoy techniques at a 1% false discovery rate.

Statistical and bioinformatic analysis—Results from the separate datasets were saved in Excel as .txt format and were uploaded into the Partek Genomics Suite 6.5 software package. The data was normalized to log base 2 and compared using 1-way ANOVA analysis as previously described [71, 75, 76]. All proteins with non-significant (p>0.05) changes were eliminated from the table. The significant values were mapped using the cluster based on significant genes' visualization function with the standardization option chosen. Principal component analysis was performed using Qlucore Omics Explorer 3.2 software.

### REFERENCES

1. Singh, A. D., M. E. Turell, and A. K. Topham, Uveal melanoma: trends in incidence, treatment, and survival. Ophthalmology, 2011. 118(9): p. 1881-5.
2. Singh, A. D., L. Bergman, and S. Seregard, Uveal melanoma: epidemiologic aspects. Ophthalmol Clin North Am, 2005. 18(1): p. 75-84, viii.
3. Raivio, I., Uveal melanoma in Finland. An epidemiological, clinical, histological and prognostic study. Acta Ophthalmol Suppl, 1977(133): p. 1-64.
4. Shammas, H. F. and F. C. Blodi, Prognostic factors in choroidal and ciliary body melanomas. Arch Ophthalmol, 1977. 95(1): p. 63-9.
5. Damato, B., Developments in the management of uveal melanoma. Clin Exp Ophthalmol, 2004. 32(6): p. 639-47.
6. Bell, D. J. and M. W. Wilson, Choroidal melanoma: natural history and management options. Cancer Control, 2004. 11(5): p. 296-303.
7. Carvajal, R. D., et al., Metastatic disease from uveal melanoma: treatment options and future prospects. Br J Ophthalmol, 2017. 101(1): p. 38-44.
8. Barker, C. A. and A. K. Salama, New NCCN Guidelines for Uveal Melanoma and Treatment of Recurrent or Progressive Distant Metastatic Melanoma. J Natl Compr Canc Netw, 2018. 16(5S): p. 646-650.
9. Shields, J. A., Current approaches to the diagnosis and management of choroidal melanomas. Surv Ophthalmol, 1977. 21(6): p. 443-63.
10. Augsburger, J. J., et al., Fine needle aspiration biopsy in the diagnosis of intraocular cancer. Cytologic-histologic correlations. Ophthalmology, 1985. 92(1): p. 39-49.
11. Sisley, K., et al., Abnormalities of chromosomes 3 and 8 in posterior uveal melanoma correlate with prognosis. Genes Chromosomes Cancer, 1997. 19(1): p. 22-8.
12. Prescher, G., et al., Prognostic implications of monosomy 3 in uveal melanoma. Lancet, 1996. 347(9010): p. 1222-5.
13. Aalto, Y., et al., Concomitant loss of chromosome 3 and whole arm losses and gains of chromosome 1, 6, or 8 in metastasizing primary uveal melanoma. Invest Ophthalmol Vis Sci, 2001. 42(2): p. 313-7.
14. Van Raamsdonk, C. D., et al., Mutations in GNA11 in uveal melanoma. N Engl J Med, 2010. 363(23): p. 2191-9.
15. Van Raamsdonk, C. D., et al., Frequent somatic mutations of GNAQ in uveal melanoma and blue naevi. Nature, 2009. 457(7229): p. 599-602.
16. Harbour, J. W., et al., Recurrent mutations at codon 625 of the splicing factor SF3B1 in uveal melanoma. Nat Genet, 2013. 45(2): p. 133-5.
17. Decatur, C. L., et al., Driver Mutations in Uveal Melanoma: Associations With Gene Expression Profile and Patient Outcomes. JAMA Ophthalmol, 2016. 134(7): p. 728-33.

18. Velez, G., et al., Personalized Proteomics for Precision Health: Identifying Biomarkers of Vitreoretinal Disease. Transl Vis Sci Technol, 2018. 7(5): p. 12.
19. Field, M. G., et al., PRAME as an Independent Biomarker for Metastasis in Uveal Melanoma. Clin Cancer Res, 2016. 22(5): p. 1234-42.
20. Onken, M. D., et al., Collaborative Ocular Oncology Group report number 1: prospective validation of a multi-gene prognostic assay in uveal melanoma. Ophthalmology, 2012. 119(8): p. 1596-603.
21. Onken, M. D., et al., An accurate, clinically feasible multi-gene expression assay for predicting metastasis in uveal melanoma. J Mol Diagn, 2010. 12(4): p. 461-8.
22. Force, A. O. O. T., International Validation of the American Joint Committee on Cancer's 7th Edition Classification of Uveal Melanoma. JAMA Ophthalmol, 2015. 133(4): p. 376-83.
23. Mellen, P. L., S. J. Morton, and C. L. Shields, American joint committee on cancer staging of uveal melanoma. Oman J Ophthalmol, 2013. 6(2): p. 116-8.
24. Mashayekhi, A., et al., Extraocular Extension of Ciliochoroidal Melanoma after Transscleral Fine-Needle Aspiration Biopsy. Retin Cases Brief Rep, 2016. 10(3): p. 289-92.
25. Schefler, A. C., et al., Extraocular extension of uveal melanoma after fine-needle aspiration, vitrectomy, and open biopsy. JAMA Ophthalmol, 2013. 131(9): p. 1220-4.
26. Raja, V., et al., Extraocular seeding of choroidal melanoma after a transretinal biopsy with a 25-gauge vitrector. Retin Cases Brief Rep, 2011. 5(3): p. 194-6.
27. Rumer, K. K., et al., Siglec-6 is expressed in gestational trophoblastic disease and affects proliferation, apoptosis and invasion. Endocr Relat Cancer, 2012. 19(6): p. 827-40.
28. Yu, Y., et al., Functional Inhibitory Siglec-6 Is Upregulated in Human Colorectal Cancer-Associated Mast Cells. Front Immunol, 2018. 9: p. 2138.
29. Richards, C. D., The enigmatic cytokine oncostatin m and roles in disease. ISRN Inflamm, 2013. 2013: p. 512103.
30. Junk, D. J., et al., Oncostatin M promotes cancer cell plasticity through cooperative STAT3-SMAD3 signaling. Oncogene, 2017. 36(28): p. 4001-4013.
31. Miller, D. M., et al., c-Myc and cancer metabolism. Clin Cancer Res, 2012. 18(20): p. 5546-53.
32. Yasuda, A., et al., The stem cell factor/c-kit receptor pathway enhances proliferation and invasion of pancreatic cancer cells. Mol Cancer, 2006. 5: p. 46.
33. Cardoso, H. J., M. I. Figueira, and S. Socorro, The stem cell factor (SCF)/c-KIT signalling in testis and prostate cancer. J Cell Commun Signal, 2017. 11(4): p. 297-307.
34. Chen, W., et al., Cancer statistics in China, 2015. CA Cancer J Clin, 2016. 66(2): p. 115-32.
35. Wang, M., et al., FABP1 Polymorphisms Contribute to Hepatocellular Carcinoma Susceptibility in Chinese Population with Liver Cirrhosis: A Case-Control Study. J Cancer, 2018. 9(22): p. 4294-4300.
36. Hui, R. W. H., et al., Inverse relationship between hepatic steatosis and hepatitis B viremia: Results of a large case-control study. J Viral Hepat, 2018. 25(1): p. 97-104.
37. Xiong, J., et al., Non-alcoholic steatohepatitis-related liver cirrhosis is increasing in China: a ten-year retrospective study. Clinics (Sao Paulo), 2015. 70(8): p. 563-8.
38. Wu, Y. L., et al., Hepatitis B Virus X Protein Induces Hepatic Steatosis by Enhancing the Expression of Liver Fatty Acid Binding Protein. J Virol, 2016. 90(4): p. 1729-40.
39. Ku, C. Y., et al., Liver fatty acid-binding protein (L-FABP) promotes cellular angiogenesis and migration in hepatocellular carcinoma. Oncotarget, 2016. 7(14): p. 18229-46.
40. Hong, I. S., Stimulatory versus suppressive effects of GM-CSF on tumor progression in multiple cancer types. Exp Mol Med, 2016. 48(7): p. e242.
41. Urdinguio, R. G., et al., Immune-dependent and independent antitumor activity of GM-CSF aberrantly expressed by mouse and human colorectal tumors. Cancer Res, 2013. 73(1): p. 395-405.
42. Yamashita, Y., N. Nara, and N. Aoki, Antiproliferative and differentiative effect of granulocyte-macrophage colony-stimulating factor on a variant human small cell lung cancer cell line. Cancer Res, 1989. 49(19): p. 5334-8.
43. Hodi, F. S., et al., Ipilimumab plus sargramostim vs ipilimumab alone for treatment of metastatic melanoma: a randomized clinical trial. JAMA, 2014. 312(17): p. 1744-53.
44. Zhao, Z., et al., Depletion of DNMT3A suppressed cell proliferation and restored PTEN in hepatocellular carcinoma cell. J Biomed Biotechnol, 2010. 2010: p. 737535.
45. Saito, Y., et al., Increased protein expression of DNA methyltransferase (DNMT) 1 is significantly correlated with the malignant potential and poor prognosis of human hepatocellular carcinomas. Int J Cancer, 2003. 105(4): p. 527-32.
46. Metcalf, D., The colony-stimulating factors and cancer. Cancer Immunol Res, 2013. 1(6): p. 351-6.
47. Yan, B. C., et al., Arginase-1: a new immunohistochemical marker of hepatocytes and hepatocellular neoplasms. Am J Surg Pathol, 2010. 34(8): p. 1147-54.
48. Arlauckas, S. P., et al., Arg1 expression defines immunosuppressive subsets of tumor-associated macrophages. Theranostics, 2018. 8(21): p. 5842-5854.
49. Bastidas, J. A., et al., The effect of pancreatic polypeptide infusion on glucose tolerance and insulin response in longitudinally studied pancreatitis-induced diabetes. Surgery, 1990. 107(6): p. 661-8.
50. Bordi, C., et al., Pancreatic polypeptide-related tumors. Peptides, 2002. 23(2): p. 339-48.
51. Friesen, S. R., J. R. Kimmel, and T. Tomita, Pancreatic polypeptide as screening marker for pancreatic polypeptide apudomas in multiple endocrinopathies. Am J Surg, 1980. 139(1): p. 61-72.
52. Demkova, L. and L. Kucerova, Role of the HGF/c-MET tyrosine kinase inhibitors in metastasic melanoma. Mol Cancer, 2018. 17(1): p. 26.
53. Abrahams, V. M., M. Kamsteeg, and G. Mor, The Fas/Fas ligand system and cancer: immune privilege and apoptosis. Mol Biotechnol, 2003. 25(1): p. 19-30.
54. Cullen, S. P., M. Brunet, and S. J. Martin, Granzymes in cancer and immunity. Cell Death Differ, 2010. 17(4): p. 616-23.
55. Brown, L., et al., Desmoglein 3 promotes cancer cell migration and invasion by regulating activator protein 1 and protein kinase C-dependent-Ezrin activation. Oncogene, 2014. 33(18): p. 2363-74.
56. Umezu-Goto, M., et al., Autotaxin has lysophospholipase D activity leading to tumor cell growth and motility by lysophosphatidic acid production. J Cell Biol, 2002. 158(2): p. 227-33.
57. Jansen, S., et al., Proteolytic maturation and activation of autotaxin (NPP2), a secretedmetastasis-enhancing lysophospholipase D. J Cell Sci, 2005. 118(Pt 14): p. 3081-9.
58. Nam, S. W., et al., Autotaxin (NPP-2), a metastasis-enhancing motogen, is an angiogenic factor. Cancer Res, 2001. 61(18): p. 6938-44.
59. Yang, S. Y., et al., Expression of autotaxin (NPP-2) is closely linked to invasiveness of breast cancer cells. Clin Exp Metastasis, 2002. 19(7): p. 603-8.
60. Yang, Y., et al., Autotaxin expression in non-small-cell lung cancer. Am J Respir Cell Mol Biol, 1999. 21(2): p. 216-22.
61. Kehlen, A., et al., Expression, regulation and function of autotaxin in thyroid carcinomas. Int J Cancer, 2004. 109(6): p. 833-8.
62. Singh, A. D., et al., Reduced expression of autotaxin predicts survival in uveal melanoma. Br J Ophthalmol, 2007. 91(10): p. 1385-92.
63. Enninga, E. A., et al., Galectin-9 modulates immunity by promoting Th2/M2 differentiation and impacts survival in patients with metastatic melanoma. Melanoma Res, 2016. 26(5): p. 429-41.
64. Wierenga, A. P. A., et al., Aqueous Humor Biomarkers Identify Three Prognostic Groups in Uveal Melanoma. Invest Ophthalmol Vis Sci, 2019. 60(14): p. 4740-4747.
65. Zhang, B., S. Kirov, and J. Snoddy, WebGestalt: an integrated system for exploring gene sets in various biological contexts. Nucleic Acids Res, 2005. 33(Web Server issue): p. W741-8.
66. Garcia-Campos, M. A., J. Espinal-Enriquez, and E. Hernandez-Lemus, Pathway Analysis: State of the Art. Front Physiol, 2015. 6: p. 383.
67. Papastefanou, V. P., et al., Metabolic activity of primary uveal melanoma on PET/CT scan and its relationship with monosomy 3 and other prognostic factors. Br J Ophthalmol, 2014. 98(12): p. 1659-65.
68. Cai, L., et al., Gene Expression Profiling and PRAME Status Versus Tumor-Node-Metastasis Staging for Prognostication in Uveal Melanoma. Am J Ophthalmol, 2018. 195: p. 154-160.
69. Harbour, J. W., et al., Are Risk Factors for Growth of Choroidal Nevi Associated With Malignant Transformation? Assessment With a Validated Genomic Biomarker. Am J Ophthalmol, 2019. 197: p. 168-179.
70. Gezgin, G., et al., PRAME as a Potential Target for Immunotherapy in Metastatic Uveal Melanoma. JAMA Ophthalmol, 2017. 135(6): p. 541-549.
71. Velez, G., et al., Precision Medicine: Personalized Proteomics for the Diagnosis and Treatment of Idiopathic Inflammatory Disease. JAMA Ophthalmol, 2016. 134(4): p. 444-8.
72. Vizcaino, J. A., et al., 2016 update of the PRIDE database and its related tools. Nucleic Acids Res, 2016. 44(D1): p. D447-56.
73. Perez-Riverol, Y., et al., PRIDE Inspector Toolsuite: Moving Toward a Universal Visualization Tool for Proteomics Data Standard Formats and Quality Assessment of ProteomeXchange Datasets. Mol Cell Proteomics, 2016. 15(1): p. 305-17.
74. Deutsch, E. W., et al., The ProteomeXchange consortium in 2017: supporting the cultural change in proteomics public data deposition. Nucleic Acids Res, 2017. 45(D1): p. D1100-D1106.
75. Velez, G., et al., Therapeutic drug repositioning using personalized proteomics of liquid biopsies. JCI Insight, 2017. 2(24).
76. Roybal, C. N., et al., Personalized Proteomics in Proliferative Vitreoretinopathy Implicate Hematopoietic Cell Recruitment and mTOR as a Therapeutic Target. Am J Ophthalmol, 2018. 186: p. 152-163.

1. A method of diagnosing and treating uveal melanoma in a patient, the method comprising:
a) obtaining a vitreous sample from an eye of the patient;
b) measuring levels of expression of one or more biomarkers selected from the group consisting of fatty acid-binding protein 1 (FABP1), granulocyte-macrophage colony-stimulating factor receptor (GM-CSF Ra), kallikrein 7 (KLK7), sialic acid-binding Ig-like lectin 6 (SIGL6), Myc proto-oncogene protein (MYC), oncostatin-M (OSM), stem cell growth factor receptor Kit (SCFR/KIT), colony stimulating factor 2 common beta chain (CSF2RB), hepatocyte growth factor receptor (c-MET/HGFR), sirtuin-1 (SIR1), granzyme A (GRAA), myocyte-specific enhancer factor 2C (MEF2C), arginase-1 (ARGI1), fas ligand (FASLG), pancreatic prohormone (PP/PAHO), DNA (cytosine-5)-methyltransferase 3A (DNMT3A), desmoglein-3 (DSG3), autotaxin (ENPP2), galectin-9 (LEG9), and hepatocyte growth factor (HGF) in the vitreous sample, wherein differential expression of FABP1, GM-CSF Ra, KLK7, SIGL6, MYC, OSM, SCFR/KIT, CSF2RB, c-MET/HGFR, SIR1, GRAA, MEF2C, ARGI1, FASLG, PP/PAHO, DNMT3A, DSG3, ENPP2, LEG9, and HGF compared to reference value ranges for a vitreous sample from a control subject indicate that the patient has uveal melanoma; and
c) treating the patient for the uveal melanoma, if the patient has a positive diagnosis for said uveal melanoma based on the levels of expression of the one or more biomarkers.

2. The method of claim 2, wherein increased levels of expression of SIGL6, c-MYC, OSM, and SCFR/c-Kit and decreased levels of expression of FABP1, GM-CSF Ra, and KLK7 compared to reference value ranges for the biomarkers in a vitreous sample from a control subject indicate that the patient has uveal melanoma.

3. The method of claim 1, further comprising classifying the uveal melanoma by gene expression profile (GEP) class if the patient has a positive diagnosis for uveal melanoma by comparing the levels of expression of one or more biomarkers selected from Table 3 and Table 6 in the vitreous sample from the eye of the patient to reference value ranges for the one or more biomarkers obtained from one or more reference vitreous samples from one or more reference subjects having uveal melanoma that has been classified by gene expression profile (GEP) class.

4. The method of claim 3, wherein the one or more biomarkers are selected from the group consisting of oncostatin M (OSM), colony stimulating factor 2 common beta chain (CSF2RB), GM-CSF Ra, FABP1, kallikrein 7, oligodendrocyte-myelin glycoprotein (OMgp), sirtuin 1, siglec-6, myocyte-specific enhancer factor 2C (MEF2C), arginase-1, DNA (cytosine-5)-methyltransferase 3A (DNMT3A), and heparin-binding EGF-like growth factor (HB-EGF).

5. The method of claim 4, wherein the one or more biomarkers comprise or consist of oncostatin M (OSM), colony stimulating factor 2 common beta chain (CSF2RB), GM-CSF Ra, FABP1, kallikrein 7, oligodendrocyte-myelin glycoprotein (OMgp), sirtuin 1, siglec-6, myocyte-specific enhancer factor 2C (MEF2C), arginase-1, DNA (cytosine-5)-methyltransferase 3A (DNMT3A), and heparin-binding EGF-like growth factor (HB-EGF).

6. The method of claim 3, wherein increased levels of expression of colony stimulating factor 2 common beta chain (CSF2RB), hepatocyte growth factor receptor (c-MET/HGFR), sirtuin-1, granzyme A, myocyte-specific enhancer factor 2C (MEF2C), arginase-1, Fas ligand (FASL), pancreatic prohormone (PP), and DNA (cytosine-5)-methyltransferase 3A (DNMT3A) compared to reference value ranges for a vitreous sample from a control subject indicate that the patient has GEP Class 2 uveal melanoma.

7. The method of claim 1, further comprising classifying the uveal melanoma by PRAME status if the patient has a positive diagnosis for uveal melanoma by comparing the levels of expression of one or more biomarkers selected from Table 4 and Table 7 in the vitreous sample from the eye of the patient to reference value ranges for the one or more biomarkers obtained from one or more reference vitreous samples from one or more reference subjects having uveal melanoma that has been classified by PRAME status.

8. The method of claim 7, wherein the one or more biomarkers are selected from the group consisting of desmoglein-3, GM-CSF Ra, FABP1, kallikrein 7, and siglec-6.

9. The method of claim 8, wherein the one or more biomarkers comprise or consist of desmoglein-3, GM-CSF Ra, FABP1, kallikrein 7, and siglec-6.

10. The method of claim 7, wherein increased levels of expression of desmoglein-3, autotaxin, galectin-9, hepatocyte growth factor (HGF), neogenin (NEO1), and pro-low-density lipoprotein receptor-related protein 1 (LRP1) indicate that the patient has PRAME positive uveal melanoma.

11. The method of claim 1, wherein the patient has been diagnosed with idiopathic uveitis.

12. The method of claim 1, further comprising detecting leukocytes in the vitreous humor or active chorioretinal inflammation in the patient.

13. The method of claim 1, wherein said treating the patient for uveal melanoma comprises administering adjuvant systemic therapy, radioactive plaque therapy, external beam proton therapy, laser therapy, enucleation, evisceration, exenteration, iridectomy, choroidectomy, iridocyclectomy, eyewall resection, chemotherapy, brachytherapy, transpupillary thermotherapy, resection of the eye tumor, gamma knife stereotactic radiosurgery, or a combination thereof.

14. The method of claim 1, wherein said measuring the levels of expression comprises performing mass spectrometry, tandem mass spectrometry, liquid chromatography, liquid chromatography-tandem mass spectrometry (LC-MS/MS), NMR, an enzyme-linked immunosorbent assay (ELISA), a radioimmunoassay (RIA), an immunofluorescent assay (IFA), immunohistochemistry, fluorescence-activated cell sorting (FACS), or a Western Blot.

15. The method of claim 14, wherein the ELISA is performed using a multiplex ELISA array.

16. (canceled)

17. The method of claim 1, further comprising genotyping the patient to determine if the patient has one or more chromosomal abnormalities or mutations linked to uveal melanoma.

18. (canceled)

19. A method of subtyping uveal melanoma and determining risk of metastasis, the method comprising:
a) obtaining a vitreous sample from an eye of a patient who has uveal melanoma;
b) measuring levels of expression of one or more biomarkers selected from the group consisting of colony stimulating factor 2 common beta chain (CSF2RB), hepatocyte growth factor receptor (c-MET/HGFR), sirtuin-1, granzyme A, myocyte-specific enhancer factor 2C (MEF2C), arginase-1, Fas ligand (FASL), pancreatic prohormone (PP), and DNA (cytosine-5)-methyltransferase 3A (DNMT3A), wherein increased levels of expression of the one or more biomarkers selected from the group consisting of CSF2RB, c-MET/HGFR, sirtuin-1, granzyme A, MEF2C, arginase-1, FASL, PP, DNMT3A in the vitreous sample from the patient compared to reference value ranges for the biomarkers from a vitreous sample from a control subject indicate that the patient has GEP class 2 uveal melanoma and is at risk of metastasis; and
c) measuring levels of expression of one or more biomarkers selected from the group consisting of desmoglein-3, autotaxin, galectin-9, hepatocyte growth factor (HGF), neogenin (NEO1), and pro-low-density lipoprotein receptor-related protein 1 (LRP1) wherein increased levels of expression of the one or more biomarkers selected from the group consisting of desmoglein-3, autotaxin, galectin-9, HGF, NEO1, and LRP1 in the vitreous sample from the eye of the patient compared to reference value ranges for the biomarkers from a vitreous sample from a control subject indicate that the patient has PRAME positive uveal melanoma and is at risk of metastasis.

20. The method of claim 19, further comprising administering adjuvant systemic therapy, radiotherapy, or performing surgery if the patient is diagnosed with GEP class 2 uveal melanoma or PRAME positive uveal melanoma.

21. The method of claim 19, further comprising measuring levels of one or more additional biomarkers selected from Table 3, Table 4, Table 6, and Table 7.

22. A method of monitoring uveal melanoma in a patient, the method comprising:
a) obtaining a first vitreous sample from an eye of the patient at a first time point and a second vitreous sample from the eye of the subject later at a second time point;
b) measuring one or more biomarkers in the first vitreous sample and the second vitreous sample, wherein the biomarkers are selected from the group consisting of SIGL6, c-MYC, OSM, SCFR/c-Kit, FABP1, KLK7, GM-CSF Ra, and serpin 1; and
c) analyzing the levels of expression of the one or more biomarkers in conjunction with respective reference value ranges for said biomarkers, wherein detection of increased levels of expression of SIGL6, c-MYC, OSM, and SCFR/c-Kit and decreased levels of expression of FABP1, KLK7, GM-CSF Ra, and serpin 1 in the second vitreous sample compared to the first vitreous sample indicate that the patient is worsening, and detection of decreased levels of expression of SIGL6, c-MYC, OSM, and SCFR/c-Kit and increased levels of expression of FABP1, KLK7, GM-CSF Ra, and serpin 1 in the second vitreous sample compared to the first vitreous sample indicate that the patient is improving.

23-39. (canceled)
